(19) United States
(12) Patent Application Publication
(10) Pub. No.: US 2014/0206624 A1
(43) Pub. Date: Jul. 24, 2014

(54) TREATMENT OF LEUKEMIA

(71) Applicants: Stephen Matthew Sykes, West Roxbury, MA (US); David T. Scadden, Weston, MA (US); Dwight Gary Gilliland, Wellesley, MA (US)

(72) Inventors: Stephen Matthew Sykes, West Roxbury, MA (US); David T. Scadden, Weston, MA (US); Dwight Gary Gilliland, Wellesley, MA (US)

(73) Assignees: THE BRIGHAM AND WOMEN'S HOSPITAL, INC., Boston, MA (US); THE GENERAL HOSPITAL CORPORATION, Boston, MA (US)

(21) Appl. No.: 13/983,325

(22) PCT Filed: Feb. 6, 2012
(86) PCT No.: PCT/US12/23928

§ 371 Date: Mar. 13, 2014

Related U.S. Application Data
(60) Provisional application No. 61/439,583, filed on Feb. 4, 2011.
(60) Provisional application No. 61/499,388, filed on Jun. 21, 2011.

(51) Int. Cl. A61K 31/713; A61K 45/06; A61K 31/436

(52) U.S. Cl. CPC A61K 31/713; A61K 31/436; A61K 45/06
USPC 514/19.6; 514/44 A; 514/44 R

(57) ABSTRACT

Compositions and methods for the treatment of leukemia by inhibiting FOXO signaling are described.

### CROSS REFERENCE TO RELATED APPLICATIONS

This application claims benefit under 35 U.S.C. §119(e) of U.S. Provisional Application No. 61/439,583 and No. 61/499,388 filed Feb. 4, 2011 and Jun. 21, 2011, respectively, the contents of which are incorporated herein by reference in their entirety.

### GOVERNMENT SUPPORT

This invention was made with Government support under Grant Nos.: 5T32CA081156, R01HL097794 and T32HL07623 awarded by the National Institutes of Health. The Government has certain rights in the invention.

### FIELD OF THE INVENTION

This invention relates to the treatment of leukemia.

### BACKGROUND OF THE INVENTION

Leukemias are malignant neoplasms of hematopoietic tissues. These neoplasms are categorized into two predominant forms: chronic and acute. While acute leukemias are characterized by undifferentiated cell populations, chronic leukemias usually present a more mature morphology. Notwithstanding these classifications, however, the pathological impairment of normal hematopoiesis is the hallmark of all leukemias

Chronic lymphocytic leukemia (CLL) is a neoplasm characterized by the clonal expansion of small lymphocytes, which accumulate in the marrow, lymph nodes, blood, spleen, liver, and other organs. The CLL cell is the neoplastic counterpart of an immunologically immature, incompetent lymphocyte. In over 95 percent of cases, the clonal expansion is of a B cell lineage. See e.g., Cancer: Principles & Practice of Oncology (3rd Edition) (1989) (pp. 1843-1847). In less than 5 percent of CLL cases, the tumor cells have a T-cell phenotype.

CLL, while accounting for only about 0.8 percent of all cancers in the United States, is the most prevalent leukemia afflicting adults in modern countries, accounting for 30 percent of all leukemias. Chronic lymphocytic leukemia affects only adults. The average age of patients is about 70 and it is rarely seen in people under the age of 40.

Acute myeloid leukemias (AMLs) are genetically heterogeneous malignant neoplasms that are associated with a low survival rate relative to most cancer types (Frohling et al., 2005). The prognosis of AML patients is dependent on the cytogenetic and molecular profile of AML cells. For instance, AML harboring t(15;17), t(8;21) or inv(1;6) (causing the fusion oncoproteins PML-RARa, AML1-ETO or CBFβ-MYH11 respectively) is associated with a favorable outcome (low risk), whereas those possessing mixed lineage leukemia (MLL)-translocations (involving chromosome 11q23) experience a poorer prognosis (high risk) and show resistance to conventional chemotherapy (Armstrong et al., 2003; Daser and Rabbitts, 2005; Dash and Gilliland, 2001).

Thus, investigation of the molecular mechanisms governing the aggressive nature of MLL-rearranged and other high-risk AMLs is needed to develop more effective therapies.

### SUMMARY OF THE INVENTION

The methods and compositions described herein are based, in part, on the surprising and counterintuitive observation that FOXO activation plays a role in leukemia onset and progression. It is generally agreed that activation of FOXO or inhibition of Akt is an important treatment in angiogenic tumors (e.g., solid tumors), however the data provided herein show that inhibition of FOXO, rather than activation, is protective in the setting of leukemias, such as acute myeloid leukemia (AML). Provided herein are methods and compositions for the treatment of leukemia by inhibiting FOXO activity, expression or subcellular localization.

One aspect described herein relates to a method of treating leukemia in a mammal in need thereof, the method comprising administering an effective amount of an agent that inhibits the activity or expression of a FOXO transcription factor in the mammal, whereby leukemia is treated in the mammal.

Also described herein in another aspect is a method of treating leukemia in a mammal in need thereof, the method comprising measuring the expression or activity of a FOXO transcription factor, and comparing the measured expression or activity of the FOXO transcription factor to a standard, wherein if the level of FOXO expression or activity is below the level of the standard, one proceeds to administer an effective amount of an agent that inhibits the expression or activity of a JNK protein kinase in a mammal, or rapamycin or a combination thereof, whereby leukemia is treated in the mammal.

In another embodiment of this aspect and all other aspects described herein, the method of treating leukemia in a mammal further comprises measuring the expression or activity of AKT protein kinase, and comparing the measured expression or activity of the AKT protein kinase to a standard, wherein if the level of AKT protein kinase expression or activity is above the level of the standard, one proceeds to administer an effective amount of an agent that inhibits the expression or activity of the JNK protein kinase in a mammal and an effective amount of rapamycin.

Also described herein in another aspect is a method of inhibiting leukemia initiating cells (LICs) in a mammal in need thereof, the method comprising administering an effective amount of an agent that inhibits the activity or expression of a FOXO transcription factor in the mammal, wherein the mammal has been diagnosed with leukemia and whereby the LICs are inhibited in the mammal.

Also described herein in another aspect is a method of inhibiting LICs in a mammal in need thereof, the method comprising measuring the expression or activity of a FOXO transcription factor, and comparing the measured expression or activity of the FOXO transcription factor to a standard, wherein if the level of FOXO expression or activity is below the level of the standard, one proceeds to administer an effective amount of an agent that inhibits the expression or activity of a JNK protein kinase in a mammal, or rapamycin or a combination thereof, whereby LICs are inhibited in the mammal.

In another embodiment of this aspect and all other aspects described herein, the method of inhibiting LICs further comprises measuring the expression or activity of AKT protein kinase, and comparing the measured expression or activity of the AKT protein kinase to a standard, wherein if the level of AKT protein kinase expression or activity is above the level of the standard, one proceeds to administer an effective amount of an agent that inhibits the expression or activity of the JNK protein kinase in a mammal and an effective amount of rapamycin.

In addition, described herein in another aspect is an agent that inhibits the activity or expression of a FOXO transcription factor for use in the treatment of leukemia in a mammal in need thereof.

In another aspect described herein is an agent that inhibits the activity or expression of a FOXO transcription factor for use in inhibiting leukemia initiating cells for the treatment of leukemia in a mammal in need thereof.

In one embodiment, provided herein is a composition comprising an agent that inhibits the activity or expression of a FOXO transcription factor for use in the treatment of leukemia in a mammal in need thereof.

In another embodiment of this aspect and all other aspects described herein, the composition further comprises an agent that inhibits the expression or activity of a JNK protein kinase in a mammal.

In another embodiment of this aspect and all other aspects described herein, the composition further comprises an agent that increases the expression or activity of the AKT protein kinase.

In one embodiment of this aspect and all other aspects described herein, the leukemia is selected from the group consisting of: acute lymphoid leukemia (ALL); chronic lymphocytic leukemia (CLL); acute myeloid leukemia (AML); chronic myeloid leukemia (CML); and mixed lineage leukemia (MLL).

In another embodiment of this aspect and all other aspects described herein, the FOXO transcription factor is FOXO 1, FOXO 3, FOXO 4 or FOXO 6.

In another embodiment of this aspect and all other aspects described herein, the agent that inhibits the activity or expression of a FOXO transcription factor is selected from the group consisting of a small molecule, an aptamer, a peptide, a protein, and an oligonucleotide.

In another embodiment of this aspect and all other aspects described herein, the agent inhibits the activity of the FOXO transcription factor in the mammal.

In another embodiment of this aspect and all other aspects described herein, the agent inhibits the expression of the FOXO transcription factor in the mammal.

In another embodiment of this aspect and all other aspects described herein, the agent increases translocation of the FOXO transcription factor out of the nucleus.

In another embodiment of this aspect and all other aspects described herein, the agent that inhibits the activity or expression of a FOXO transcription factor is an RNAi agent that inhibits the expression of the FOXO transcription factor in the mammal.

In another embodiment of this aspect and all other aspects described herein, the RNAi agent comprises a double-stranded ribonucleic acid (dsRNA) wherein the dsRNA comprises a sense strand and an antisense strand, the antisense strand comprising a region of complementarity to and having at least 15 contiguous nucleotides differing by no more than 3 nucleotides from a molecule selected from the group consisting of SEQ ID NOs: 11-22.

In another embodiment of this aspect and all other aspects described herein, the RNAi agent comprises the nucleotide sequence of one of SEQ ID NOs: 11-14.

In another embodiment of this aspect and all other aspects described herein, the agent that inhibits the activity or expression of a FOXO transcription factor is a vector comprising a nucleic acid sequence that is at least 90% identical to one of SEQ. ID. Nos. 11-22.

In another embodiment of this aspect and all other aspects described herein, the vector is a viral vector.

In another embodiment of this aspect and all other aspects described herein, the mammal is a human.

In another embodiment of this aspect and all other aspects described herein, the method of treatment of leukemia or inhibiting leukemia initiating cells further comprises the steps, before the administering step, of measuring the expression or activity of a FOXO transcription factor, and comparing the measured expression or activity of the FOXO transcription factor to a standard, wherein if the level of FOXO expression or activity is above the level of the standard, one proceeds to the administering step.

In another embodiment of this aspect and all other aspects described herein, the method further comprises administering an effective amount of an agent that increases the expression or activity of the AKT protein kinase in the mammal.

In another embodiment of this aspect and all other aspects described herein, the agent that increases the expression or activity of the AKT protein kinase is selected from the group consisting of a small molecule, an aptamer, a peptide, a protein, and an oligonucleotide.

In another embodiment of this aspect and all other aspects described herein, the agent that increases the expression or activity of the AKT protein kinase is a vector comprising a sequence encoding an AKT protein kinase.

In another embodiment of this aspect and all other aspects described herein, the vector is an expression vector comprising a nucleic acid sequence that is at least 95% identical to SEQ. ID. No. 23.

In another embodiment of this aspect and all other aspects described herein, the agent that increases the expression or activity of the AKT protein kinase is an activator of AKT activity selected from the group consisting of rapamycin, CCI-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, insulin growth factor-1, platelet derived growth factor and granulocyte colony-stimulating factor (G-CSF).

In another embodiment of this aspect and all other aspects described herein, the method of treating leukemia or inhibiting leukemia initiating cells further comprises administering an effective amount of an agent that inhibits the expression or activity of a JNK protein kinase.

In another embodiment of this aspect and all other aspects described herein, the agent that inhibits activity of JNK protein kinase is 4-[9-(tetrahydro-furan-3-yl)-8-(2,4,6-trifluoro-phenylamino)-9H-purin-2-ylamino]-cyclohexan-1-ol (JNK 930/CC-930) or 1-(5-(1H-1,2,4-triazol-5-yl)(1H-indazol-3-yl))-3-(2-piperidylethoxy)benzene (JNK 401/CC-401).

In another embodiment of this aspect and all other aspects described herein, the method of treating leukemia or inhibiting leukemia initiating cells further comprises administering an effective amount of rapamycin.

In another embodiment of this aspect and all other aspects described herein the method of treating leukemia or inhibiting leukemia initiating cells further comprises measuring the expression or activity of AKT protein kinase, and comparing the measured expression or activity of the AKT protein kinase to a standard, wherein if the level of AKT protein kinase expression or activity is above the level of the standard, one proceeds to administer an effective amount of an agent that inhibits the expression or activity of the JNK protein kinase in a mammal and an effective amount of rapamycin.

In another embodiment of this aspect and all other aspects described herein, the method of treatment of leukemia or inhibiting leukemia initiating cells further comprises administering an effective amount of an agent commonly used in treating leukemia, such as cytarabine (ara-C) and an anthracycline drug, e.g., daunorubicin (daunomycin) or idarubicin.

In another embodiment of this aspect and all other aspects described herein, the method of treatment of leukemia or inhibiting leukemia initiating cells further comprises administering an effective amount of a TGF-beta inhibitor such as AP 12009 and MYO-029.

### DEFINITIONS

As used herein, the term “FOXO transcription factor” refers to a transcription factor within the 0 subclass of forkhead transcription factors, which are typically characterized by a fork head DNA binding domain. Some examples of FOXO transcription factors and their nucleic acid sequences include FOXO1 (GENBANK™ Accession No. NM_{—}002015; SEQ. ID. NO:1), FOXO3 (GENBANK™ Accession Nos. NM_{—}001455.3 (variant 1; SEQ. ID. NO:2) or NM_{—}201559.2 (variant 2; SEQ. ID. NO: 3), FOXO4 (GENBANK™ Accession Nos. NM_{—}001170931.1 (isoform 1; SEQ. ID. NO: 4) or NM_{—}005938.3 (isoform 2; SEQ. ID. NO: 5)), and FOXO6 (GENBANK™ Accession Nos. XM_{—}002346379.1 (predicted; SEQ. ID. NO: 6) or XM_{—}002342102.1 (predicted; SEQ. ID. NO: 7)). In one embodiment, the FOXO transcription factor associated with or influenced by the methods and compositions described herein is a human FOXO transcription factor.

As used herein, the term “inhibits the activity or expression of a FOXO transcription factor” or “inhibiting the activity of a FOXO transcription factor” refers to a decrease in the activity (e.g., as measured by subcellular localization of FOXO or phosphorylation status of a FOXO transcription factor or DNA binding capability and subsequent expression of a downstream target of FOXO such as e.g., Cdkn1b (p27), Cited2, Ccrn4l, Meis1, Tmem71 or Ccng2) or expression levels of a FOXO protein (e.g., as measured by ELISA, Western Blot, RT-PCR etc.) or a FOXO nucleic acid by at least 10% in a cell population treated with a FOXO inhibitor compared to the activity or expression levels of the FOXO protein in the cell population prior to treatment with a FOXO inhibitor. In one embodiment, the decrease in activity or expression levels of a FOXO protein by a FOXO inhibitor (interchangeably referred to herein as an agent that inhibits the activity or expression of a FOXO transcription factor) is produced through direct interaction with a FOXO gene (including regulatory regions thereof), a FOXO transcript, or partial or full-length FOXO polypeptide sequence. Included, for example, is an anti-FOXO antibody or antigen-binding fragment thereof that binds specifically to a FOXO polypeptide. Alternatively, the levels of FOXO activity or expression in a cell population treated with a FOXO inhibitor can be compared to a standard (e.g., an untreated control cell population, a reference value, or a population standard). In other embodiments, the activity or expression level of FOXO is inhibited by at least 20%, at least 30%, at least 40%, at least 50%, at least 60%, at least 70%, at least 80%, at least 90%, at least 95%, at least 99% or even 100% (i.e., FOXO activity or expression is below detectable levels or is absent). In one embodiment, the cell population is a population in a mammal. In another embodiment, the cell population is a population of cells in a human.

As used herein, the term “increases translocation of the FOXO transcription factor out of the nucleus” refers to an increase in the ratio of the level of FOXO present in the cytoplasm to the level of FOXO present in the nucleus by at least 5%; preferably the ratio is increased by at least 10%, at least 20%, at least 30%, at least 40%, at least 50%, at least 60%, at least 70%, at least 80%, at least 90%, at least 95%, at least 99%, at least 1-fold, at least 5-fold, at least 10-fold, at least 20-fold, at least 50-fold, at least 100-fold, at least 1000-fold or more. In one embodiment, there is no detectable level of FOXO in the nucleus and essentially all of the FOXO is detectable in the cytoplasm of the cell. One of skill in the art will be aware that FOXO is rapidly degraded once it translocates into the cytoplasm. Given the rapid degradation of cytoplasmic FOXO, the measured level of FOXO in the cytoplasm will underestimate the level of FOXO in that compartment. Thus, in one embodiment, the amount of FOXO in the nucleus is compared to the total FOXO level in the cell.

As used herein, the terms “JNK protein kinase” or “JNK” refers to a c-Jun N-terminal kinase that binds and phosphorylates c-Jun on Ser-63 and Ser-73 within its transcriptional activation domain. Some examples of JNK protein kinase and their nucleic acid sequences include human JNK1 (GenBank Accession No. L26318), human JNK1-al (GenBank Accession No. NM_{—}002750.2), human JNK2 (GenBank Accession No. U09759.1) and human JNK3 (GenBank Accession No. U07620). In one embodiment, the JNK protein kinase associated with or influenced by the methods and compositions described herein is a human JNK protein kinase.

As used herein, the term “inhibits the activity or expression a JNK protein kinase” or “inhibiting the activity of a JNK protein kinase” refers to a decrease in the activity (e.g., as measured by phosphorylation status of a downstream target of JNK protein kinase such as e.g., c-Jun) or expression levels of a JNK protein kinase (e.g., as measured by ELISA, Western Blot, RT-PCR etc.) or a JNK nucleic acid by at least 10% in a cell population treated with a JNK inhibitor compared to the activity or expression levels of the JNK protein in the cell population prior to treatment with a JNK inhibitor. In one embodiment, the decrease in activity or expression levels of a JNK protein kinase by a JNK inhibitor (interchangeably referred to herein as an agent that inhibits the activity or expression of a JNK protein kinase) is produced through direct interaction with a JNK gene (including regulatory regions thereof), a JNK transcript, or partial or full-length JNK polypeptide sequence. For example, an anti-FOXO antibody or antigenic fragment thereof binding specifically to a FOXO polypeptide. Alternatively, the levels of JNK activity or expression in a cell population treated with a JNK protein kinase inhibitor can be compared to a standard (e.g., an untreated control cell population, a reference value, or a population standard). In other embodiments, the activity or expression level of JNK protein kinase is inhibited by at least 20%, at least 30%, at least 40%, at least 50%, at least 60%, at least 70%, at least 80%, at least 90%, at least 95%, at least 99% or even 100% (i.e., JNK protein kinase activity or expression is below detectable levels or is absent). In one embodiment, the cell population is a population in a mammal. In another embodiment, the cell population is a population of cells in a human.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene, thereby inhibiting expression of the target gene. In one embodiment, the RNA is a double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be, for example, at least 10%, at least 20%, at least 30%, at least 40%, at least 50%, at least 60%, at least 70%, at least 80%, at least 90%, at least 95% or at least 99% or more as compared to the expression of a target gene or the activity or level of the protein encoded by a target gene which has not been targeted by an RNA interference molecule. The terms “RNA interference” and “RNA interference molecule” as they are used herein are intended to encompass those forms of gene silencing mediated by double-stranded RNA, regardless of whether the RNA interfering agent comprises an siRNA, miRNA, shRNA or other double-stranded RNA molecule. In some embodiments, the RNAi molecule further comprises modifications, such as 2′-deoxy-2′-fluorouridine or locked nucleic acid (LNA) nucleotides and RNA duplexes containing either phosphodiester or varying numbers of phosphorothioate linkages. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are commercially available. The locked ribose conformation enhances base stacking and backbone pre-organization. This significantly increases the hybridization properties (melting temperature) of oligonucleotides. Such modifications are known to one skilled in the art and are described, for example, in Braasch et al., Biochemistry 42: 7967-7975 (2003), the contents of which are incorporated herein by reference in its entirety.

“Short interfering RNA” (siRNA), also referred to herein as “small interfering RNA” is defined as an RNA agent which functions to inhibit expression of a target gene, e.g., by RNAi. An siRNA may be chemically synthesized, may be produced by in vitro transcription, or may be produced within a host cell, for example, a genetically modified host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA). In one embodiment, an RNA interference molecule is expressed from a plasmid, retrovirus, or lentivirus using, e.g., a pol III U6 promoter, or another promoter to drive expression (see, e.g., Stewart, et al. (2003) RNA Apr; 9(4):493-501, incorporated by reference herein in its entirety).

An “shRNA” or “small hairpin RNA” is a type of a double-stranded RNA interference molecule comprising a single strand of nucleic acid that folds back on itself to form a molecule having a double stranded region and a single stranded loop region (e.g., a hairpin conformation). In one embodiment, shRNAs are composed of a short, e.g. about 19 to about 25 nucleotide, antisense strand, followed by a single stranded loop generally, of about 5 to about 9 nucleotides, and the analogous sense strand (or vice versa). shRNAs can have increased stability over other RNA interfering molecules, and may be preferred in conditions that result in increased RNA interference molecule degradation in a cell.

The terms “microRNA” or “miRNA” are used interchangeably herein and are used to describe short endogenous RNAs, which are post-transcriptional regulators of mRNA expression and generally effect gene silencing. Artificial microRNAs can be synthesized and administered to a cell in order to inhibit expression of a protein from a target mRNA by mediating RNA interference. In one embodiment, a microRNA is between 11 and 22 nucleotides in length. In other embodiments, a microRNA is between 12-24, 13-23, 14-22, 15-21, 16-20, 17-19, 15-25, 17-25, 19-25, 20-25, 21-25, 22-25, 23-25, 11-15, 12-15, 13-15, or 14-18 nucleotides in length.

As used herein, the terms “treat”, “treatment” or “treating” used in reference to leukemia refers to measures that delay the onset, reverse, alleviate, ameliorate, decrease, inhibit, or slow down the progression or severity of a condition or symptom associated with leukemia. In one embodiment, the term “treating” includes reducing or alleviating at least one adverse effect or symptom of a condition, disease or disorder associated with leukemia, such as, but not limited to, high white blood cell count. In one embodiment, treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in the absence of treatment. In another embodiment, “treatment” of leukemia also includes providing relief from the symptoms or side-effects of the disease (including palliative treatment). For example, any reduction in—symptoms of leukemia—no matter how slight would be considered an alleviated symptom. In some embodiments of the aspects described herein, the symptoms or a measured parameter of leukemia are alleviated by at least 5%, at least 10%, at least 20%, at least 30%, at least 40%, at least 50%, at least 60%, at least 70%, at least 80%, or at least 90%, upon administration of an agent, as compared to a control or non-treated subject. In some embodiments, “treatment” and “treating” can also mean prolonging survival as compared to expected survival if not receiving treatment. Thus, one of skill in the art realizes that a treatment may improve the disease condition, but may not be a complete cure for the disease.

Leukemia is largely asymptomatic until later disease stages. Accordingly, in one embodiment, the terms “preventing” or “prevention” as applied to the prevention of leukemia includes slowing the development of all bodily symptoms due to leukemia, wherein bodily symptoms are the manifestation of the later disease stages. For example, poor platelet count, poor clotting ability, being easily bruised, and having an enlarged spleen to name a few. That is, in one embodiment, “preventing” or “prevention” means preventing the onset of detectable symptoms of leukemia or keeping the patient largely asymptomatic as long as possible.

The term “reduced”, “reduce” or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. substantially absent or below levels of detection), or any decrease between 10-100% as compared to a reference level, as that term is defined herein.

As used herein, the term “standard” or “reference” refers to the amount or activity of a FOXO transcription factor, a JNK signaling molecule or an AKT signaling molecule (including e.g., activity, expression or amount in the cytoplasm vs. the nucleus) in a known sample against which another sample is compared; alternatively, a standard can simply be a reference number that represents an amount of FOXO, JNK or AKT expression or activity and that defines a baseline for comparison. The reference number can be derived from either a sample taken from an individual, or a plurality of individuals. That is, the “standard” does not need to be a sample that is tested, but can be an accepted reference number or value. A series of standards can be developed that take into account an individual's status, e.g., with respect to age, gender, weight, height, ethnic background etc. A standard level can be obtained, for example, from a known biological sample from a different individual who does not or is not suspected of having leukemia, or is not at risk for leukemia (e.g., not the individual being tested). A known sample can also be obtained by pooling samples from a plurality of individuals to produce a standard over an averaged population. Additionally, a standard can be synthesized such that a series of standards are used to quantify the amount of FOXO, JNK and/or AKT activity/expression or subcellular localization in an individual's sample. Alternatively, a standard can be from the same individual, having been taken at a time after the onset of leukemia. In such instances, the standard can provide a measure of the efficacy of treatment with a FOXO inhibitor, JNK inhibitor, AKT activator or other agent. In one embodiment, the standard is taken from the individual prior to any treatment for leukemia, e.g., data obtained for the diagnosis of leukemia. Ideally, a biological standard will be obtained at an earlier time point (presumably prior to the onset of leukemia or prior to onset of treatment with a FOXO inhibitor, JNK inhibitor, AKT activator or other agent) from the same individual that is to be tested or treated as described herein.

As used herein, the phrase “below the level of a standard” refers to a measured level of, e.g., FOXO or AKT activity or expression that is lower than a given standard by a statistically significant amount. As an alternative, a measured level is “below the level of” a standard where it is at least 5% lower, preferably at least 10% lower or more, e.g., at least 20%, 30%, 40%, 50%, 60%, 70% or more below the level of the standard.

As used herein, the phrase “above the level of a standard” or “greater than the level of a standard” refers to a measured level of, e.g., FOXO, AKT or JNK activity or expression that is higher than or above a given standard by a statistically significant amount. As an alternative, a measured level is “above the level of” or “greater than the level of” a standard where it is at least 5% higher, preferably at least 10% higher or more, e.g., at least 20%, 30%, 40%, 50%, 60%, 70%, 80%, 90% or more higher, e.g., one fold, 2-fold or more higher than the level of the standard.

The term “effective amount” as used herein refers to the amount of a FOXO inhibitor (e.g., a FOXO3 inhibitor) or JNK protein kinase inhibitor, or an AKT agent (e.g., an AKT activator, AKT expression vector comprising an AKT nucleic acid or AKT nucleic acid) needed to reduce or alleviate at least one or more symptoms of a leukemia, and relates to a sufficient amount of pharmacological composition to provide the desired effect, i.e., treat a subject having acute myeloid leukemia or another leukemia. In one embodiment, the term “therapeutically effective amount” therefore refers to an amount of a FOXO inhibitor, JNK protein kinase inhibitor or an AKT agent that is sufficient to provide a particular effect when administered to a typical subject, such as one who has or is at risk for a leukemia disorder. In another embodiment, an effective amount as used herein would also include an amount sufficient to prevent or delay the development of a symptom of the disease, alter the course of a symptom disease (for example but not limited to, slowing the progression of a symptom of the disease), or reverse a symptom of the disease. Thus, it is not possible or practical to specify an exact “effective amount” for every instance. However, for any given case, an appropriate “effective amount” can be determined by one of ordinary skill in the art using no more than routine experimentation.

An “Akt nucleic acid”, as used herein, refers to a nucleic acid molecule which codes for an Akt polypeptide (i.e., a polypeptide that phosphorylates FOXO transcription factors), and in particular, inhibits self-renewal of leukemia-initiating cells (LICs). In one embodiment, a nucleic acid encoding a constitutively active form of Akt having the amino acid sequence of SEQ ID NO. 26 is used with the methods described herein. Akt nucleic acids also include homologs and alleles of a nucleic acid having the sequence of one of SEQ ID NOs. 23-25, as well as functionally equivalent fragments, variants, and analogs of the foregoing nucleic acids. “Functionally equivalent”, in reference to an Akt nucleic acid fragment, variant, or analog, refers to a nucleic acid that codes for an Akt polypeptide that inhibits LIC self-renewal. Preferably the Akt polypeptide variant, analog or derivative maintains serine-threonine kinase activity. Methods and compositions for modulating Akt activity in a cell can be found in e.g., U.S. Pat. No. 7,368,420, which is herein incorporated by reference in its entirety.

In one embodiment, as used herein, the term “leukemia initiating cells (LICs)” refers to a subset of cells that possess the ability to initiate, maintain and serially propagate leukemia (e.g., AML) in vivo, while retaining the ability to differentiate into more committed progeny that lack these properties. Progenies of LIC that give rise to AML are immature normal cells change into cancer cells and they do not mature before entering into circulation. Instead, these perpetually immature cancer cells move into the blood circulation and also pile up in the bone marrow. In another embodiment, LICs are CD34+ myeloid progenitors having an immunophenotype comprising lineage^{low}, cKit^{hi}, Sca-1⁻, FcγRII/III⁺ and further comprising leukemia-initiating cell activity. In some embodiments, the lineage^{low} immunophenotype is determined using a lineage cocktail comprising antibodies targeting CD3, CD4, CD8, CD19, B220, Gr-1, Ter119, and IL-7Rα.

As used herein, the term “leukemia-initiating cell activity” refers to the activity of a leukemia-initiating cell to undergo self-renewal and further to initiate and/or sustains leukemic symptoms, and/or continued production of immature cancer cells described. In one embodiment LIC activity is determined by measuring the disease initiation rate in an animal model of LIC transplant where low LIC activity is associated with impaired disease initiation. For example, a competitive or non-competitive bone marrow repopulation assay can be used to determine the activity of LICs. A non-competitive repopulation assay is performed by transplanting LICs into a lethally irradiated mammal, while a competitive repopulation assay involves transplanting a mixture of LICs and normal progenitors into the lethally irradiated mammal. Exemplary methods for such assays are provided herein in the Examples section or in Tothova, Z., et al. Cell 128:325-339 (2007), which is incorporated herein by reference in its entirety.

As used herein the term “inhibiting leukemia-initiating cells” refers to a reduction in the number of LICs in a cell population by at least 10% following treatment with a FOXO inhibitor as described herein. In one embodiment, the cell population is in a mammal treated with a FOXO inhibitor, a JNK inhibitor and/or an AKT agent. Preferably the number of LICs is reduced by at least 20%, at least 30%, at least 40%, at least 50%, at least 60%, at least 70%, at least 80%, at least 90%, at least 95%, at least 99%, or even 100% (i.e., below detectable levels or absent). LICs can be inhibited by any mechanism including, for example, inducing the LIC to lose its self-renewal capacity (e.g., by inducing differentiation), killing LICs (e.g., inducing apoptosis) or inducing immune cell disposal of LICs.

In one embodiment, pharmaceutical compositions for the treatment of leukemia and for the inhibition of LICs are provided herein. The pharmaceutical composition comprises the various compositions described herein and at least one pharmaceutically acceptable carrier.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. In one embodiment, a pharmaceutically acceptable carrier does not include tissue culture media. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, embodiments of the present invention were performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Molecular Biology, Vo. 203, 2003, Transgenic Mouse, edited by Marten H. Hofker and Jan van Deursen, which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIGS. 1A-1G show that constitutive AKT activation promotes myeloid maturation and apoptosis of leukemic cells.

FIG. 1A shows reduced AKT activation in CD34+ leukemic progenitor cells. Lineage^{low}, Sca-1⁻, cKit^{hi}, CD34⁺ cells purified from healthy and MLL-AF9-induced leukemic mice were subjected to flow cytometry with phospho-AKT^{Ser473} (CD34+ myeloid progenitors (MP) vs. CD34+ leukemic progenitors (LP), p=0.0478). Right panel is a histogram from a single experiment with bar graphs representing aggregate data from 3 experiments.

FIG. 1B shows that constitutive AKT activation in bone marrow cells derived from MLL-AF9-induced leukemic mouse cells results in cell growth defects. Mononuclear bone marrow (MNBC) cells recovered from MLL-AF9 leukemic mice were infected with MSCV-IRES-GFP control (Ctrl) or myrAKT-expressing retroviruses. Following infection, cells from each condition were treated with vehicle, 10 nM or 100 nM rapamycin and then cells from each condition was evaluated for GFP expression every 2 days using flow cytometry (Day 6*Ctrl vs. myrAKT, vehicle p=0.0005, **Ctrl vs. myrAKT, 10 nM rapamycin p=0.0008, ***Ctrl vs. myrAKT, 100 nM rapamycin p=0.0046; n=3).

FIG. 1C shows that constitutive AKT activation in bone marrow cells derived from MLL-AF9-induced leukemic mouse cells results in increase number of CD11b+ mature myeloid cells. GFP⁺ cells treated as described in FIG. 1B were analyzed for CD11b expression at Day 6 post-treatment using flow cytometry (*Ctrl vs. myrAKT, vehicle p<0.0001, **Ctrl vs. myrAKT, 10 nM rapamycin p<0.0001, ***Ctrl vs. myrAKT, 100 nM rapamycin p<0.0001; n=3).

FIG. 1D shows the morphological changes and granulation vesicles associated with myeloid maturation upon constitutive AKT activation in bone marrow cells derived from MLL-AF9-induced leukemic mouse cells. Untreated GFP⁺ cells from control and myrAKT-infected cells were purified by flow cytometry and then stained with May-Grünwald Giemsa.

FIGS. 1E and 1F show the phagocytic ability of mature myeloid cells induced upon constitutive AKT activation in bone marrow cells derived from MLL-AF9-induced leukemic mouse cells. GFP⁺ cells treated as described in panel B were incubated with pHrodo fluorescent-labeled E. coli particles and subsequently analyzed by flow cytometry for GFP+pHrodo+ cells. FIG. 1E shows flow cytometric histogram plot of pHrodo stained Ctrl vs. myrAKT GFP+ cells and FIG. 1F shows graphical representation of three replicates. (*Ctrl vs. myrAKT, vehicle p<0.0001, **Ctrl vs. myrAKT, 10 nM rapamycin p<0.0001, ***Ctrl vs. myrAKT, 100 nM rapamycin p=0.0009; n=3).

FIG. 1G shows that constitutive AKT activation in bone marrow cells derived from MLL-AF9-induced leukemic mouse cells results in increased apoptosis. Untreated GFP⁺ Ctrl and myrAKT-expressing cells were stained with Annexin V to assess cell death (*Ctrl vs. myrAKT p=0.0006; n=3).

FIGS. 2A-2E show that phosphorylation of AKT^{Thr308} is diminished in L-GMPs.

FIG. 2A shows reduced AKT activation, via AKT phosphorylation, in CD34+ leukemic progenitor cells. Lineage^{low}, Sca-1⁻, cKit^{hi}, CD34⁺ cells purified from healthy and MLL-AF9-induced leukemic mice were subjected to flow cytometry with phospho-AKT^{Thr308} (CD34+ myeloid progenitors (MP) vs. CD34+ leukemic progenitors (LP)) and

FIG. 2B shows reduced AKT downstream effector molecules activation, via S6 phosphorylation, in CD34+ leukemic progenitor cells. phospho-S6^{Ser235/236} (MP vs. LP p=0.0674). For B. the right panel is a histogram from a single experiment with bar graphs representing aggregate data from 2 experiments.

FIG. 2C shows that rapamycin effectively inhibits mTOR signaling in bone marrow cells derived from MLL-AF9-induced leukemic mouse cells having constitutive AKT activation. MLL-AF9-expressing leukemia BM cells were treated with 0, 1, 10 or 100 nM Rapamycin (CELL SIGNALING TECHNOLOGY®) ex vivo for 24 hours. Following treatment cells were lysed and subjected to western blot with indicated antibodies.

FIG. 2D shows that constitutive AKT activation in bone marrow cells derived from MLL-AF9-induced leukemic mouse cells results in increased mTOR substrates phosphorylation. MLL-AF9-expressing leukemia BM cells stably transduced with control or myrAKT-expressing recombinant retroviruses were lysed and subjected to western blot with the indicated antibodies.

FIG. 2E shows that constitutive AKT activation in bone marrow cells derived from MLL-AF9-induced leukemic mouse cells results in cells that are larger (forward scattering) and more granulated (side scattering). Mononuclear bone marrow (MNBC) cells recovered from MLL-AF9 leukemic mice were infected with MSCV-IRES-GFP control (Ctrl) or myrAKT-expressing retroviruses. Following infection, cells from each condition were treated with either vehicle or 1 nM rapamycin and then cells from all conditions were evaluated for size and granularity by flow cytometry six days post-infection.

FIGS. 3A-3D show that FoxO transcription factors are active and suppress myeloid maturations in murine AML cells.

FIG. 3A shows the tabulated list of previously described activated (left panel) and repressed (right panel) FoxO target genes that are significantly differentially (D-Chip analysis, p=0.95) expressed between GMP and L-GMP microarray data sets (Krivtsov et al., 2006).

FIG. 3B shows inducible knock down expression of FoxO3 upon induced expression of CreER in MNBCs leukemia cells expressing MLL-AF9, bearing floxed alleles for FoxO1, FoxO3 and FoxO4 (FoxO1/3/4^{floxed}; MLL-AF9 cells) and infected with CreER expressing recombinant retroviruses. The MNBCs leukemia cells were infected with Ctrl or CreER expressing recombinant retroviruses. Stably transduced Ctrl and CreER cells were then treated with vehicle or 400 nM 4-hydroxytamoxifen (4-OHT) for 4-6 hours. 48-72 hours following treatment cells from all conditions were harvested and subjected to western blot with FoxO3 (75D8), Tubulin and Cre antibodies.

FIG. 3C shows that inducible knock down expression of FoxO3 results in increased number of CD11b+ mature myeloid cells from MNBCs leukemia cells expressing MLL-AF9, bearing floxed alleles for FoxO1, FoxO3 and FoxO4 (FoxO1/3/4^{floxed}; MLL-AF9 cells) and infected CreER expressing recombinant retroviruses. The MNBCs leukemia cells were infected with Ctrl or CreER expressing recombinant retroviruses, and were then treated with vehicle or 4-OHT as described above and then plated in liquid culture. Five days following treatment, cells from each condition were assessed for CD11b and Gr-1 expression by flow cytometry (*CreER+4-OHT vs. CreER+vehicle, Ctrl+vehicle, or Ctrl+4-OHT p<0.0001; n=3).

FIG. 3D shows the morphological changes associated with myeloid maturation of the Ctrl and CreER cells from each treatment condition described above stained with May-Grünwald Giemsa.

FIGS. 4A-4F show that deletion of FoxO1/3/4 promotes myeloid maturation and reduces leukemic cell growth on supportive stroma.

FIG. 4A shows that deletion of FoxO1/3/4 in bone marrow cells derived from MLL-AF9-induced leukemic mouse cells results in increase number of CD11b+ mature myeloid cells. Leukemic BM cells expressing MLL-AF9 and bearing floxed alleles for FoxO1, FoxO3 and FoxO4 (FoxO1/3/4^{floxed}; MLL-AF9 cells) were infected with Ctrl or CreER expressing recombinant retroviruses. Stably transduced Ctrl and CreER cells were then treated with vehicle, 50, 100, 200 or 400 nM 4-hydroxytamoxifen (4-OHT) for 4-6 hours. Five days following treatment, cells from each condition were assessed for CD11b and Gr-1 expression by flow cytometry (*CreER+4-OHT vs. CreER+vehicle, Ctrl+vehicle, or Ctrl+4-OHT p<0.0001).

FIG. 4B shows the morphological changes associated with myeloid maturation upon FoxO1/3/4 deletion. FoxO1/3/4^{floxed}; MLL-AF9; Ctrl and FoxO1/3/4^{floxed}; MLL-AF9; CreER cells from each CreER+/−4-OHT were stained with May-Grünwald Giemsa.

FIG. 4C shows the flow cytometric plot of hCD34 expression and size, and CD11b and Gr-1 expression upon FoxO1/3/4 deletion. MLL-AF9-expressing leukemic BM was stably transduced with lentiviruses that carry a loxP-STOP-loxP-human CD34 cassette (LSL-hCD34). These cells were then transduced with control or CreER-expressing recombinant retroviruses. MLL-AF9; LSL-hCD34; CreER or control cells were treated with vehicle of 4-OHT as described above and then assessed 5 days later for mature myeloid surface marker expression, CD11b and Gr-1 by flow cytometry.

FIG. 4D shows the graphical representation of CD11b and Gr-1 expression in each condition (Ctrl and CreER) of MLL-AF9; LSL-hCD3 cells in FIG. 4C

FIG. 4E demonstrates the diminished colony forming capability of MLL-AF9/FoxO1/3/4^{floxed} cells upon FoxO1/3/4 deletion after one day. Vehicle and 4-OHT-treated FoxO1/3/4^{floxed}; MLL-AF9; CreER and control cells were layered on top of pre-plated OP-9 cells 24 hours following treatment. Colony formation was assessed after 14 days of co-culture.

FIG. 4F demonstrates the diminished colony forming capability of MLL-AF9/FoxO1/3/4^{floxed} cells upon FoxO1/3/4 deletion after 7 and 14 days. FoxO1/3/4^{floxed}; MLL-AF9; CreER and control cells were co-cultured with OP-9 stroma. Seven days later, cobblestones area forming colonies (CAFC) were counted and subsequently administered vehicle or 400 nM 4-hydroxytamoxifen (4-OHT) for 4-6 hours. Seven days following 4-OHT or vehicle treatment CAFC were again counted and tabulated.

FIGS. 5A-5G show that FOXO3 is active and required to preserve the immature state of human AML cell lines.

FIG. 5A shows that FOXO3 is predominately localized in the nucleus of human AML cell lines. MLL-AF9-positive (THP-1 and Mono-mac-6 (MM6)) and MLLAF9-negative (SKM-1 and NB4) leukemia cell lines were biochemically fractionated to recover nuclear (N) and cytoplasmic (C) extracts, which were then subjected to western blot with FoxO3-specific antibodies (75D8). Both nuclear and cytoplasmic fractions from each cell line were also subjected to western blot with Orc2-(nuclear) and Tubulin-(cytoplasmic) specific antibodies to determine the efficiency of biochemical separation.

FIG. 5B shows that shRNA targeting FOXO3 reduces expression of FOXO3 in three human AML cell lines, MOLM-14, MM6 and SKM-1. MOLM-14, MM6 and SKM-1 cells were stably transduced with recombinant lentiviruses expressing either non-targeting (NT) or FOXO3 (F3-1 or F3-2) shRNAs. Following stable infection, cells were lysed and subjected to western blot with FOXO3 and Tubulin antibodies.

FIG. 5C shows that shRNA targeting FOXO3 results in decreased cell growth in human AML cell lines, MOLM-14, MM6, SKM-1 and NB4. MOLM-14 cells were counted either everyday (SKM-1 and NB4) or every two days (MOLM-14 and MM6) following stable expression of designated shRNAs (Day 6 MOLM-14, NT vs. F3-1 *p=0.0003, NT vs. F3-2 **p<0.0001; Day 6 MM6, NT vs. F3-1 *p<0.0001, NT vs. F3-2 **p=0.0002; Day 4 SKM-1, NT vs. F3-1 *p=0.0062 NT vs. F3-2 **p=0.0083, Day 4 NB4, NT vs. F3-1 *p=0.0003 NT vs. F3-2 **p=0.0058).

FIG. 5D shows that shRNA targeting FOXO3 results in increased expression of myeloid maturation cell surface marker CD11b in human AML cell lines, MOLM-14 and SKM-1. MOLM-14 and SKM-1 cells from each shRNA condition were also analyzed for human CD11b expression by flow cytometry. Histogram plots are representative of one experiment and the bar graphs are graphical representations of three independent infections (MOLM-14, NT vs. F3-1 *p<0.0001, NT vs. F3-2 **p<0.0001; SKM-1, NT vs. F3-1 *p<0.0001 NT vs. F3-2 **p=0.0115).

FIG. 5E shows that shRNA targeting FOXO3 results in increased phagocytic activity of the human AML cell line, MM6. MM6 cells from each shRNA condition were incubated with pHrodo fluorescent-labeled E. coli particles and subsequently analyzed by flow cytometry for pHrodo+ cells.

FIG. 5F shows the morphological changes associated with myeloid maturation upon FoxO3 deletion by shRNA targeting FOXO3 in human AML cell lines, MOLM-14, SKM-1 and NB4. May-Grünwald Giemsa staining of cytospins of MOLM-14, SKM-1 and NB4 cells stably expressing either NT of FOXO3 shRNA-1 molecules

FIG. 5G shows that shRNA targeting FOXO3 results in increased apoptosis it CD11b+ cells in human AML cell line, SKM-1. SKM-1 cells from each shRNA condition were stained with Annexin V and CD11b and assessed for CD11b⁻Annexin V⁺ and CD11b⁺Annexin V⁺ staining (*NT shRNA vs. FOXO3 shRNA-1, CD11b+ p<0.0001, **NT shRNA vs. FOXO3 shRNA-2, CD11b+ p=0.0007; n=3). (NT=NT shRNA, F3-1=FOXO3 shRNA-1 and F3-2=FOXO3 shRNA-2; n=3).

FIGS. 6A-6F show that depletion of FOXO3 promotes myeloid maturation and apoptosis of human AML cells.

FIG. 6A shows enriched nuclear localization of FOXO3 in human AML cell lines. NOMO1, THP-1, U-937, NB4 and K562 were stably transduced with recombinant lentiviruses expressing either non-targeting (NT shRNA) or FOXO3 (FOXO3 shRNA-1 & -2) shRNAs. Following stable infection, cells were lysed and subjected to western blot with FOXO3 and Tubulin antibodies.

FIG. 6B shows that shRNA targeting FOXO3 results in decreased cell growth in human AML cell lines. NOMO1, THP-1, U-937, NB4 and K562 cells were counted every 1-2 days following stable expression of designated shRNAs (at Day 4 NOMO1 1 NT vs. F3-1 *p<0.0001; at Day 6 THP-1 NT vs. F3-1 *p<0.0001 1 NT vs. F3-2 **p<0.0001; at Day 4 U-937 NT vs. F3-1 *p=0.0003, NT vs. F3-2 **p<0.0001; at Day 4 NB4 NT vs. F3-1 *p=0.0003 NT vs. F3-2 **p=0.0058).

FIG. 6C shows that shRNA targeting FOXO3 results in increased expression of myeloid maturation cell surface marker CD11b in human AML cell lines. SKM-1, NB4, HL-60 and THP-1 cells expressing either NT, FOXO3-1 and FOXO3-2 shRNAs were analyzed for human CD11b expression by flow cytometry eight days post-infection (SKM-1 NT vs. F3-1 *p<0.0001, NT vs. F3-2 **p=0.0115, F. NB4 NT vs. F3-1 *p<0.0001, NT vs. F3-2 **p=0.0008 G. HL-60 NT vs. F3-1 *p=0.0005; THP-1 NT vs. F3-1 *p<0.0001, NT vs. F3-2 **p<0.0001).

FIG. 6D shows that shRNA targeting FOXO3 results in increased phagocytic activity of human AML cell lines. Mono-Mac-6 and L. K562 cells stably expressing either NT, FOXO3-1 and FOXO3-2 shRNA molecules were incubated with pHrodo fluorescent-labeled E. coli particles and subsequently analyzed by flow cytometry for pHrodo+ cells.

FIG. 6E shows the morphological changes and granulation vesicles associated with myeloid maturation upon FoxO3 deletion by shRNA targeting FOXO3 in human AML cell lines. Cytospins of NOMO1, THP-1, MonoMac-6 (MM6), U-937 and HL-60 cells were stained with May-Grünwald Giemsa 7-8 days following infection with lentiviruses expressing either NT or FOXO3-1 shRNAs.

FIG. 6F shows that shRNA targeting FOXO3 results in increased apoptosis it CD11b+ cells in human AML cell lines. THP-1, MOLM-14 and NB4 cells expressing either NT, FOXO3-1 or FOXO3-2 shRNAs were stained with Annexin V and CD11b and assessed for CD11b⁻Annexin r and CD11b⁺Annexin r staining (THP-1 *NT vs. F3-1, CD11b+ p<0.0001, **NT vs. F3-2, CD11b+ p<0.0001; MOLM-14 *NT shRNA vs. FOXO3 shRNA-1, CD11b+ p<0.0001, **NT shRNA vs. FOXO3 shRNA-2, CD11b+ p<0.0001; NB4 (*NT shRNA vs. FOXO3 shRNA-1, CD11b+ p<0.0001, **NT shRNA vs. FOXO3 shRNA-2, CD11b+ p<0.0001). (NT=NT shRNA, F3-1=FOXO3 shRNA-1 and F3-2=FOXO3 shRNA-2).

FIG. 7A-7G show that primary AML cells derived from patients separated into distinct clusters of FOXO activity.

FIG. 7A shows predominant cytoplasmic localization of FOXO3 in human AML Lin^{low} CD34⁺ cells from patients. BM cells derived from patients with AML were stained with human lineage cocktail and human CD34 (both BD Biosciences) and then lineage low, CD34+ cells were isolated by flow cytometry. Total bone marrow (TBM) cells and lineage low, CD34+ cells were biochemically fractionated to recover nuclear (N) and cytoplasmic (C) extracts, which were then subjected to western blot with FoxO3-specific antibodies (75D8). The efficiency of biochemical separation was determined with Orc2-(nuclear) and Tubulin-(cytoplasmic) specific antibodies.

FIG. 7B shows enriched nuclear localization of FOXO3 in human AML Lin^{low} CD34⁺ cells derived from patients. Lineage low, CD34+ cells from 3 patients diagnosed with AML were fractionated and analyzed as described in panel A.

FIG. 7C shows that shRNA targeting FOXO3 results in increased expression of myeloid maturation cell surface marker CD11b in patient derived AML cells. Patient samples #1 and #6 were transduced with recombinant lentiviruses expressing either NT shRNA or FOXO3 shRNA-1 and then placed in methylcellulose supplemented with human cytokines (H4034, Stem Cell Technologies). Graph represents the enumeration of colonies formed after eight days of culture.

FIG. 7D shows that shRNA targeting FOXO3 results in decreased colony forming capability in patient derived AML cells. Patient samples #1 and #6 cells expressing control and FOXO3 shRNAs were grown in liquid culture for eight days and then assessed for the mature myeloid cell marker CD11b.

FIG. 7E shows that shRNA targeting FOXO3 results in morphological changes associated with myeloid maturation in patient derived AML cells. Patient samples #1 cells expressing control and FOXO3 shRNAs were grown in liquid culture for eight days and then stained with Wright-Giemsa.

FIG. 7F shows the gene list comprising the FOXO specific gene signature generated from comparing the gene expression array data of murine lineage low, Sca-1+, cKit+ (LSK) cells in animals without (+/+) and with (Δ/Δ) FoxO1/3/4 deletion.

FIG. 7G shows the hierarchical cluster analysis based on the overlap of the murine FOXO gene signature stratified over the gene expression array data of 436 individual primary AML samples. Non-random clustering of cytogenetically defined leukemia groups indicates biological relevance (see also FIGS. 8C and 8D) (Bullinger et al., 2004; Kharas et al., 2010a).

FIGS. 8A-8D show that FOXOs are active in primary AML samples derived from patients.

FIG. 8A shows predominant cytoplasmic localization of FOXO3 in human AML Lin^{low} CD34⁺ cells and some nuclear localization of FOXO3 in human AML Lin^{low} CD34⁻ cells from patient #5. BM cells derived from patients with AML (designated as patient #5) were stained with human lineage cocktail and human CD34 (both available from BD biosciences) and then lineage low, CD34+ cells were isolated by flow cytometry. Total bone marrow (TBM) cells, lineage high, CD34- and lineage low, CD34+ cells were biochemically fractionated to recover nuclear (N) and cytoplasmic (C) extracts, which were then subjected to western blot with FoxO3-specific antibodies (75D8). Both nuclear and cytoplasmic fractions from each cell line were also subjected to western blot with Orc2-(nuclear) and Tubulin-(cytoplasmic) specific antibodies to determine the efficiency of biochemical separation.

FIG. 8B shows the localization (nuclear or cytoplasmic) of FOXO3 in human AML cells derived from patients. Whole BM aspirates (TBM) from 4 patients diagnosed with AML were fractionated and blotted as described in panel A.

FIG. 8C demonstrates the segregation of AML patients into nine AML-sub groups based on the level of FOXO3 activity (i.e., cluster 1 or 2). Distribution of the FOXO gene signature clusters among various chromosomal abnormities and geneotypes commonly seen in human AML (p<0.0001).

FIG. 8D demonstrates the segregation of AML patients in the FLT3-ITD-positive AML-sub group based on the level of FOXO3 activity (i.e., cluster 1 or 2). Distribution of FLT3-ITD-positive and negative AMLs within each FOXO gene signature defined cluster (p<0.0001)

FIGS. 9A-9I show that deletion of FoxO transcription factors of MLL-AF9-induced leukemia in vivo reduces disease burden, extends latency and diminishes the function of leukemia initiating cell (LIC) function.

FIG. 9A shows the experimental scheme used in FIGS. 9B-9H to examine the importance of FoxO1/3/4 in the maintenance of MLL-AF9-induced AML. MLL-AF9-positive FoxO1/3/4^{floxed} murine bone marrow (BM) cell recovered from a leukemic primary recipient mousecarrying the Mx1-Cre transgene (Mx1-Cre+) or not (Mx1-Cre−) were transplanted into sub-lethally irradiated secondary recipient mice. Fourteen days following transplant 50% of both Mx1-Cre+ and Mx1-Cre− mice were administered pI-pC and the remaining 50% of mice from each condition were given saline. At the first sign of leukemia all mice from each Mx1-Cre condition status were euthanized (Day 29 for Mx1-Cre−(saline and pI-pC treated) and Day 39 for Mx1-Cre+ (saline & pI-pC-treated)).

FIG. 9B shows the mean spleen weight from each condition (Mx1-Cre+ (pI-pC) vs. Mx1-Cre+ (saline), p<0.0001; n=4). Induced deletion of the FoxO1/3/4^{floxed} allele significantly reduces splenomegaly.

FIG. 9C shows the gross anatomical view of spleens recovered from FoxO1/3/4^{floxed}; Mx1-Cre+; MLL-AF9 mice administered pI-pC (right, n=2) or saline (left, n=2) 39 days post-transplant.

FIG. 9D shows that induced deletion of the FoxO1/3/4^{floxed} allele significantly reduces leukocytosis. WBC analysis of peripheral blood collected every 4-14 days post-transplant from Mx1-Cre+ saline and pI-pC-treated mice. Data is independent from Panel C (n=4).

FIG. 9E shows that FoxO1/3/4^{floxed}; Mx1-Cre−; MLL-AF9 mice have equally poor survival whether treated with saline (ctrl) or pI-pC. Kaplan-Meier survival curve analysis of recipient mice Mx1-Cre− transplanted and treated as described above; n=6, p=0.6899.

FIG. 9F shows that FoxO1/3/4 ablation prolongs survival. Kaplan-Meier survival curve analysis of recipient mice Mx1-Cre+ transplanted and treated as described above. (p=0.0009; n=10).

FIG. 9G shows that FoxO1/3/4 ablation results in reduction of L-GMP. Seven days following the administration of saline or pI-pC, secondary mice transplanted with MLL-AF9-positive FoxO1/3/4^{floxed}; Mx1-Cre+ leukmic BM cells were euthanized. Leukemic BM cells were then isolated from each treatment condition (Saline (Ctrl) versus pI-pC (pI-pC)) were analyzed for the proportion of L-GMPs (lineage^{low}, Sca-1⁻, cKit^{hi}, CD34⁺, FcgRII/III⁺, p=0.039; n=3).

FIGS. 9H and 9I demonstrate that FoxO1/3/4 ablation results in increased average survival because of a reduction in the LIC frequency. Using the same experimental set-up described in panels G leukemia BM cells Saline (MLL-AF9; FoxO1/3/4^{+/+}, panel H) versus pI-pC (MLL-AF9; FoxO1/3/4^{Δ/Δ}, panel I) were by isolated by flow cytometry and subsequently transplanted into tertiary recipients at various cell numbers: 300 (n=6), 3,000 (n=6), 30,000 (n=4) and 300,000 (n=4). Animals were then monitored weekly and euthanized upon presentation of external (moribund) and internal (e.g. WBC) signs of AML. The LIC frequencies of each condition are located in the bottom left corner (H. LICfreq^{+/+}=1:20,688 and I. LICfreq^{Δ/Δ}=1: 269,310).

FIG. 10A-10D show the excision efficiency of pI-pC treatment in FoxO1/3/4^{floxed}; MLL-AF9; Mx1-Cre+ mice.

FIG. 10A shows the effectiveness of FoxO1/3/4 ablation in leukocytosis. WBC analysis of peripheral blood recovered from mice examined in FIGS. 5B and 5C (Mx1-Cre+ (pI-pC) vs. Mx1-Cre+ (saline), p<0.0001; n=4).

FIG. 10B shows the schematic (left) experimental outline of the Kaplan-Meier survival curve analysis of mice transplanted with FoxO1/3/4^{floxed}; Mx1-Cre+ leukmic BM cells and treated with pI-pC. As described in FIG. 5, MLL-AF9-induced AML was generated in a FoxO1/3/4^{floxed}; Mx1-Cre+ genetic background that was subsequently transplanted into secondary mice. Fourteen days post-transplant, secondary recipients were administered with saline or pI-pC and monitored for the development of AML (FIG. 5). BM cells recovered from saline (Ctrl) and pI-pC treated mice that succumbed to AML were then transplanted into tertiary recipients and evaluated for the development of AML (p=0.0061; n=8).

FIGS. 10C-10E show the suppression of FOXO1/3/4 expression respectively upon treatment with pI-pC. RNA recovered from BM was converted to cDNA and then analyzed for FoxO1 (FIG. 10C.), FoxO3 (FIG. 10D.), FoxO4 (FIG. 10E.) and GAPDH expression by quantitative Real Time PCR. Each FoxO gene expression is normalized with GAPDH expression.

FIG. 10F is a Western blot showing below detection amount of FOXO3 upon treatment with pI-pC. BM recovered from one saline-treated FoxO1/3/4^{floxed}; MLL-AF9; Mx1-Cre+ mouse (lane 1) five FoxO1/3/4^{floxed}; MLL-AF9; Mx1-Cre+ mice treated with pI-pC (lanes 2-6) that succumbed to AML-related death were subjected to Western blot with FoxO3 (75D8) and Tubulin antibodies.

FIG. 10G shows that FoxO1/3/4 ablation results in an increase of lineage^{low}, CD11b^{hi} myeloid cells. Seven days following the administration of saline or pI-pC, secondary mice transplanted with MLL-AF9-positive FoxO1/3/4^{floxed}; Mx1-Cre+ leukemic BM cells were euthanized. Leukemic BM cells were then isolated from each treatment condition (Saline (Ctrl) versus pI-pC (pI-pC)) were analyzed for the proportion of mature myeloid cells (lineage^{hi}, CD11b⁺, p=0.0098; n=3)

FIG. 10H shows that FoxO1/3/4 ablation provides insignificant survival latency to AML. BM cells recovered from MLL-AF9 leukemic mice were infected with MSCV-IRES-GFP control (Ctrl) or myrAKT-expressing retroviruses. Following infection, 100,000 GFP+ cells from each condition were purified and separately injected into syngeneic recipients. Mice were monitored for AML-related death.

FIG. 11A-11F show that the JNK/c-JUN signaling pathway antagonizes maturation and apoptosis mediated by FOXO inhibition in AML.

FIG. 11A shows increased activation of the JNK pathway, demonstrated via phosphorylation of JNK, upon shRNA knock down expression of FOXO3 in AML cell line. MOLM-14 cells stably expressing either non-targeting (NT) or FOXO3 (FOXO3-1) shRNA were lysed and subjected to western blot with antibodies that specifically recognize FOXO3, c-JUN, JNK, Tubulin or the phosphorylated forms of c-JUN (pc-JUN^{S63}) and JNK (pJNK^{T183/Y185}).

FIG. 11B shows that pharmacological inhibition of JNK significantly reduced AML cell growth in the absence of FOXO3. MOLM-14 cells stably expressing non-targeting (NT) or FOXO3 (F3-1) shRNA were treated with 10 μM SP600125 (JNK inhibitor) or vehicle. Forty-eight hours after treatment cells from each condition were assessed for cell number (B., NT (vehicle) vs. NT (SP600125) *p=0.0095, NT (SP600125) vs. F3-1 (vehicle) *p=0.0008, F3-1 (vehicle) vs. F3-1 (SP600125) ***p<0.0001) and Annexin V staining.

FIG. 11C shows that pharmacological inhibition of JNK significantly increases apoptosis in AML cells in the absence of FOXO3. NT (vehicle) vs. NT (SP600125) *p=0.0282, NT (SP600125) vs. F3-1 (vehicle) *p=0.0133, F3-1 (vehicle) vs. F3-1 (SP600125) ***p=0.0002).

FIG. 11D shows increased activation of the JNK pathway, demonstrated via phosphorylation of JNK, upon constitutive activation of AKT in AML cell line. MLL-AF9-expressing leukemia BM cells stably transduced with control or myrAKT-expressing recombinant retroviruses were lysed and subjected to western blot with antibodies that specifically recognize FoxO3, c-JUN, Jnk, Tubulin or the phosphorylated forms of FoxO3 (pFoxO3^{S256}) c-JUN (pc-JUN^{S63}) and Jnk (pJnk^{T183/Y185}).

FIG. 11E shows that pharmacological inhibition of JNK significantly reduced cell growth in AML cell lines in the presence of constitutive active AKT. MLL-AF9-expressing leukemia BM cells stably transduced with control or myrAKT-expressing recombinant retroviruses were treated with 10 μM SP600125 (JNK inhibitor) or vehicle. Forty-eight hours after treatment cells from each condition were assessed for cell number (E., Ctrl (vehicle) vs. Ctrl (SP600125) *p=0.0373, Ctrl (vehicle) vs. myrAKT (vehicle) **p=0.0381, myrAKT (vehicle) vs. myrAKT (SP600125) ***p<0.0001).

FIG. 11F shows that pharmacological inhibition of JNK significantly increased apoptosis of AML cells in the presence of constitutive active AKT. Ctrl (SP600125) vs. myrAKT (vehicle) *p=0.0011, myrAKT (vehicle) vs. myrAKT (SP600125) **p<0.0001) and Annexin V staining.

FIGS. 12A-12F show that the JNK inhibitor, SP600125 cooperates with AKT activation or FOXO inhibition in driving myeloid maturation.

FIG. 12A are Western blots showing increased JNK phosphorylation upon shRNA targeted knock down expression of FOXO3-1 in AML cell lines. MOLM-14 and SKM-1 cells stably expressing either non-targeting (NT) or FOXO3 (FOXO3-1) shRNA were lysed and subjected to western blot with the indicted antibodies.

FIG. 12B are Western blots showing substantial increase in phosphorylated JNK and total c-JUN levels upon shRNA targeted knock down expression of FOXO3-1 in AML cell line. SKM-1 cells stably expressing either non-targeting (NT) or FOXO3 (FOXO3-1) shRNA were lysed and subjected to western blot with the indicted antibodies.

FIG. 12C shows increased apoptosis upon combined JNK inhibition and shRNA targeted knock down expression of FOXO3-1 in AML cell line. SKM-1 cells stably expressing non-targeting (NT) or FOXO3 (F3-1) shRNA were treated with 10 μM SP600125 (JNK inhibitor) or vehicle. Forty-eight hours after treatment cells from each condition were assessed for Annexin V staining (NT (SP600125) vs. F3-1 (vehicle) *p<0.0001, F3-1 (vehicle) vs. F3-1 (SP600125) **p=0.0001).

FIG. 12D shows increased expression of mature myeloid surface marker upon combined JNK inhibition and shRNA targeted knock down expression of FOXO3-1 in AML cell line. MOLM-14 and SKM-1 cells stably expressing non-targeting (NT) or FOXO3 (F3-1) shRNA were treated with 10 μM SP600125 (JNK inhibitor) or vehicle. Forty-eight hours after treatment cells from each condition were assessed for CD11b expression (MOLM-14 NT (SP600125) vs. F3-1 (vehicle) *p=0.0008, F3-1 (vehicle) vs. F3-1 (SP600125) **p<0.0001; SKM-1 NT (SP600125) vs. F3-1 (vehicle) *p<0.0001, F3-1 (vehicle) vs. F3-1 (SP600125) **p=0.0001).

FIG. 12E shows increased expression of mature myeloid surface marker upon combined JNK inhibition and constitutive AKT activation. MLL-AF9-expressing leukemia BM cells stably transduced with control or myrAKT-expressing recombinant retroviruses were treated with 10 μM SP600125 (JNK inhibitor) or vehicle. Forty-eight hours after treatment cells from each condition were assessed CD11b and Gr-1 expression.

FIG. 12F shows the changes in morphology and cell surface marker expressions associated with myeloid maturation upon combined JNK inhibition and constitutive AKT activation or FOXO3 expression knock down. GFP+, CD11b^{int}, Gr-1^{int} cells were purified by flow cytometry from vehicle- and SP600125-treated control cells as well as SP600125-treated myrAKT-positive cells. Additionally, flow cytometry was used to isolate GFP+, CD11b⁺, Gr-1^{int} cells from vehicle- and SP600125-treated myrAKT-positive cells and GFP+, CD11b⁺, Gr-1⁺ cells from SP600125-treated myrAKT-positive cells. Purified cells were subjected to cytospin followed by Wright-Giemsa staining.

FIGS. 13A-13D show that c-JUN activity is up-regulated in AMLs displaying constitutive AKT activation or FOXO inhibition.

FIG. 13A shows that FoxO1/3/4 ablation in FoxO1/3/4^{floxed}; MLL-AF9; Mx1-Cre+ mice results in increased phosphorylation of c-Jun. BM cells recovered from mice that succumbed to control or myrAKT-expressing MLL-AF9-induced AML (refer to FIG. 10G) were subjected to western blot with antibodies that specifically recognize AKT, Tubulin or the phosphorylated forms of FoxO3 (pFoxO3^{S256}) c-JUN (pc-JUN^{S63}).

FIG. 13B shows that constitutive activation of AKT results in increased phosphorylation of c-Jun. BM cells recovered from mice that succumbed MLL-AF9-induced AML (refer to FIG. 5F) that were wild type or null for FoxO1/3/4 were subjected to western blot with antibodies that specifically recognize AKT, Tubulin or the phosphorylated forms of FoxO3 (pFoxO3^{S256}) c-JUN (pc-JUN^{S63}).

FIG. 13C shows the mean expression levels of FOXO1, FOXO3 and c-JUN in the FOXO signature-based, hierarchical cluster-defined primary AML sample groups (p<0.0001; see FIG. 7G).

FIG. 13D shows a model depicting the biological and molecular consequences of AKT activation or FOXO inhibition in AML cells.

### DETAILED DESCRIPTION OF THE INVENTION

The study described herein sought to define, in detail, AKT/FOXO signaling in AML using an established murine model. Unexpectedly, low levels of AKT activity were associated with elevated levels of FOXOs required to maintain the function and immature state of leukemia-initiating cells (LICs). Furthermore, depletion of FOXO3 promoted differentiation and subsequent apoptosis of human myeloid leukemia cells. These data reveal an unrecognized role of the AKT/FOXO signaling pathway in the regulation and maintenance of AML that runs counter to the established roles of AKT/FOXO signaling in human cancer. Furthermore, these findings identify specific molecular targets for differentiation-inducing approaches to leukemia.

Accordingly, the present invention relates to methods of treating leukemia in a subject via a counterintuitive method involving modulation of the AKT/FOXO signaling pathway. Embodiments of the present invention are based on the discovery that constitutively active AKT in mixed lineage leukemia (MLL AF9-leukemic) cell lines severely reduced cell growth in vitro, greatly promoted terminal myeloid differentiation of these cells and also greatly increased apoptosis of the differentiated cells. In addition, the inventors also discovered that the deletion of FOXO3 transcription factor promoted differentiation and apoptosis of the MLL cell line. These observations were not unique to the MLL cell line. The inventors showed that shRNA-mediated depletion of FOXO3 promoted terminal differentiation of not only human AML cell lines expressing MLL-AF9 (THP-1, Monomac-6, MOLM-14 and NOMO-1) but also human APL cell lines (HL60 & NB4) and human cell lines that did not display complex cytogenetics (U-937 & SKM-1). The cell lines described herein are representative of various leukemia disorders, thus permitting one of skill in the art to apply the methods and compositions described herein to the treatment of a variety of leukemias from different origins. These discoveries indicate that constitutive activation of AKT or reduction or deletion of FOXO activity reduces leukemogenesis by promoting terminal differentiation of leukemic blasts into mature monocytes.

The inventors have found that activation of the putative oncogene AKT or inhibition of the FOXO family of tumor suppressor genes counterintuitively suppresses the growth of human Acute Myeloid Leukemia (AML) cells. These observations support the notion that molecules that either activate AKT and/or inhibit FOXOs have efficacy in the treatment of human AML patients. While not wishing to be bound by theory, inhibiting FOXO promotes maturation and differentiation of the immature myeloid progenitor cells to into its designated developmental pathway which ultimately leads to apoptosis. This in turns reduces leukemogenesis. Similarly, activation of AKT serves to indirectly inhibit FOXO by phosphorylating FOXO and thereby promoting FOXO degradation in the cytoplasm and preventing of FOXO entry into the nucleus.

In addition, the inventors also found AML cells with activated AKT, reduced amount of FOXO, or active FOXOs was associated activated the JNK pathway. Therefore, the combination of the activation of AKT and/or the inhibition of FOXOs together with a concurrent inhibition of a JNK protein kinase have efficacy in the treatment of human AML patients.

Accordingly, in one embodiment, provided herein is a composition for the treatment of leukemia in a mammal, the composition comprising an agent that inhibits the activity or expression of a FOXO transcription factor.

In one embodiment, the agent that inhibits the activity or expression of a FOXO transcription factor can be a small molecule, an oligonucleotide or an RNAi agent against a FOXO 1, FOXO3, FOXO4 or FOXO6 transcription factor. In one embodiment, the agent is an RNAi agent targeting the transcripts SEQ. ID. NOS: 1-7.

In another embodiment, the RNAi agent comprises a double-stranded ribonucleic acid (dsRNA) wherein the dsRNA comprises a sense strand and an antisense strand, the antisense strand comprising a region of complementarity to and having at least 15 contiguous nucleotides differing by no more than 3 nucleotides from a molecule selected from the group consisting of SEQ ID NOs:11-22.

In another embodiment, the RNAi agent comprises the nucleotide sequence of one of SEQ ID NOs: 11-14.

In another embodiment, the RNAi agent comprises the nucleotide sequence of one of SEQ ID NOs: 11-14.

In another embodiment, the agent that inhibits the activity or expression of a FOXO transcription factor is a vector comprising a nucleic acid sequence that is at least 90% identical to one of SEQ. ID. Nos. 11-22.

In one embodiment, more than one FOXO transcription factor can be inhibited in the mammal by the composition, for example, FOXO 1 and FOXO 3, FOXO 4 and FOXO6, or FOXO 1, FOXO3 and FOXO4. Accordingly, in one embodiment, provided herein is a composition for the treatment of leukemia in a mammal, the composition comprises at least one agent that inhibits the activity or expression of a FOXO transcription factor. For example, a composition comprising one agent inhibiting the activity or expression of FOXO 1 and a second agent inhibiting the activity or expression of FOXO3; or a composition comprising one agent inhibiting the activity or expression of FOXO 1, a second agent inhibiting the activity or expression of FOXO3, and a third agent inhibiting the activity or expression of FOXO4.

In another embodiment, provided herein is a composition for the treatment of leukemia in a mammal, the composition comprising an agent that increases the activity or expression of the AKT protein kinase in the mammal for the treatment of leukemia. This agent that increases the activity or expression of the AKT protein kinase can be of a small molecule, an aptamer, a peptide, a protein, and an oligonucleotide.

In one embodiment, the agent that increases the expression or activity of the AKT protein kinase is a vector comprising a sequence encoding an AKT protein kinase.

In one embodiment, the vector is an expression vector comprising a nucleic acid sequence that is at least 95% sequence identical to SEQ. ID. No. 23.

In one embodiment, the agent that increases the expression or activity of the AKT protein kinase is a small molecule activator of AKT activity and is selected from the group consisting of rapamycin, CCl-779, nicotine, Ro-31-8220, carbachol, 4-(methylnitrosamino)-1-(3-pyridyl)-1-butanone

(NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, insulin-like growth factor-1, platelet derived growth factor and granulocyte colony-stimulating factor (G-CSF).

In one embodiment, the AKT protein kinase activity can be activated and the expression of the AKT protein kinase can be increased simultaneously in the mammal for treatment purposes. Accordingly, in one aspect, described herein is a composition for the treatment of leukemia in a mammal, the composition comprising at least an agent that increases the activity of the AKT protein and at least an agent the increases the expression of the AKT kinase in the mammal for the treatment of leukemia. For example, the composition comprises an expression vector comprising a nucleic acid sequence that is at least 95% sequence identical to SEQ. ID. No. 23 and G-CSF. It is contemplated herein that there can be more than one small molecule activator of AKT activity in the composition.

In another embodiment, provided herein is a composition for the treatment of leukemia in a mammal, the composition comprising an agent that inhibits the activity or expression of a FOXO transcription factor and an agent that increases the activity or expression of the AKT protein kinase in the mammal for the treatment of leukemia. In one embodiment, the composition comprises at least an agent that inhibits the activity or expression of a FOXO transcription factor and at least an agent that increases the activity or expression of the AKT protein kinase in the mammal for the treatment of leukemia.

In another embodiment, provided herein is a composition for the treatment of leukemia in a mammal, the composition comprising an agent that inhibits the activity or expression of a FOXO transcription factor and an agent that inhibits the activity or expression of a JNK protein kinase in the mammal for the treatment of leukemia. In another embodiment, the composition comprises at least an agent that inhibits the activity or expression of a FOXO transcription factor and at least an agent that inhibits the activity or expression of a JNK protein kinase in the mammal for the treatment of leukemia.

In another embodiment, provided herein is a composition for the treatment of leukemia in a mammal, the composition comprising an agent that inhibits the activity or expression of a FOXO transcription factor, an agent that increases the activity or expression of the AKT protein kinase and an agent that inhibits the activity or expression of a JNK protein kinase in the mammal for the treatment of leukemia. In another embodiment, the composition comprises at least an agent that inhibits the activity or expression of a FOXO transcription factor, at least an agent that increases the activity or expression of the AKT protein kinase and at least an agent that inhibits the activity or expression of a JNK protein kinase in the mammal for the treatment of leukemia.

In one embodiment, the agent that inhibits activity of JNK protein kinase is 4-[9-(tetrahydro-furan-3-yl)-8-(2,4,6-trifluoro-phenylamino)-9H-purin-2-ylamino]-cyclohexan-1-ol (JNK 930/CC-930) or 1-(5-(1H-1,2,4-triazol-5-yl)(1H-indazol-3-yl))-3-(2-piperidylethoxy)benzene (JNK 401/CC-401).

In another embodiment of any aspects of the compositions described herein, the composition further comprises at least one commonly known leukemia treatment drug, e.g., cytarabine (ara-C) and an anthracycline drug, e.g., daunorubicin (daunomycin) or idarubicin. In yet another embodiment of any aspects of the compositions described herein, the composition further comprises a TGF-beta inhibitor such as AP 12009 and MYO-029. In yet another embodiment of any aspects of the compositions described herein, the composition further comprises an agent that can relieve undesired symptoms associated with leukemia. For example, relieving symptoms of nausea and fever.

In addition, accordingly, embodiments of the present invention provide methods of treating leukemia in a mammal in need thereof, for example, by administering an effective amount of an agent that inhibits the activity or expression of a FOXO transcription factor in the mammal, whereby leukemia is treated in the mammal.

In one aspect described herein, embodied herein is a method of treating leukemia in a mammal in need thereof, the method comprising administering an effective amount of any one of the compositions described herein to the mammal, whereby leukemia is treated in the mammal.

In one aspects of the methods described herein, the method further comprising measurement of the expression or activity of a FOXO transcription factor prior to use of the agent or composition comprising the agent, and comparing the measured expression or activity of the FOXO transcription factor to a standard, wherein if the level of FOXO expression or activity is above the level of the standard, one proceeds with use of the agent or medicament comprising the agent.

In one embodiment, in practicing any one of aspects of the described methods, the standard or reference level of FOXO is that normally found in a population of bone marrow progenitor cells (e.g., mononuclear bone marrow (MNBC) cells) from a healthy subject who do not have leukemia or has not been diagnosed with leukemia. In another embodiment of any one of aspects of the described methods, the standard or reference level of FOXO is that normally found in a population of white blood cells obtain from the peripheral circulation of a healthy subject who do not have leukemia or has not been diagnosed with leukemia. It is also contemplated that the standard or reference level of FOXO is the average of the standards or reference levels of FOXO obtain from a population of healthy subjects who do not have leukemia or have not been diagnosed with leukemia.

The standard or reference level can be that one particular FOXO transcription factor, for example, FOXO 1, FOXO3, FOXO4 and FOXO6. In one embodiment, the standard or reference level is the combination of those of two or more particular FOXO transcription factors. For example, FOXO 1 and FOXO3, FOXO 1 and FOXO4, FOXO 1 and FOXO6, FOXO3 and FOXO4, FOXO4 and FOXO6, FOXO3 and FOXO6, FOXO 1, FOXO3 and FOXO6, FOXO 1, FOXO3 and FOXO4, FOXO3, FOXO4 and FOXO6, or all the FOXO transcription factors FOXO 1, FOXO3, FOXO4 and FOXO6.

In one embodiment, after a subject (e.g., human) has been diagnosed with leukemia but before any treatment, the subject's bone marrow progenitor cells are assessed to determine the level of FOXO. If the FOXO level is above the standard described herein, e.g. 5% above the standard or above the standard by two standard deviations (SD) from the population mean or average, then the prescribed treatment comprises inhibiting the activity or expression of FOXO, and/or activating AKT is administered.

In one embodiment, after a subject (e.g., human) has been diagnosed with leukemia and treated for leukemia for a period of time, the subject's bone marrow progenitor cells or white blood cells are assessed again to determine the level of FOXO. This post-treatment FOXO level is then compared with the pre-treatment FOXO level of the same subject to assess the efficacy of the leukemia treatment. If the FOXO level is reduced compared to the standard described herein, e.g. 5% reduction, then the prescribed treatment is deemed effective.

In one embodiment, in practicing any one of aspects of the described methods, after the subject have been treated for leukemia for a period of time, the subject's bone marrow progenitor cells or white blood cells are assessed to determine the activity level of the c-Jun pathway, e.g., determining the level of phosphorylated c-Jun and other signaling molecules in are activated by JNK protein kinase in the JNK pathway, these signaling molecules are known in the art. The activity level of the c-Jun pathway is compared to a standard. If the level of c-Jun pathway is above the level of the standard, one proceeds to administer an effective amount of an agent that inhibits the expression or activity of the JNK protein kinase in a mammal and an effective amount of rapamycin.

In one embodiment, in practicing any one of aspects of the described methods, the standard or reference level of the c-Jun pathway is that normally found in a population of bone marrow progenitor cells (e.g., mononuclear bone marrow (MNBC) cells) from a healthy subject who do not have leukemia or has not been diagnosed with leukemia. In another embodiment of any one of aspects of the described methods, the standard or reference level of the c-Jun pathway is that normally found in a population of white blood cells obtain from the peripheral circulation of a healthy subject who do not have leukemia or has not been diagnosed with leukemia. It is also contemplated that the standard or reference level of the c-Jun pathway is the average of the standards or reference levels of the c-Jun pathway obtain from a population of healthy subjects who do not have leukemia or have not been diagnosed with leukemia.

Also described herein in another aspect is a method of treating leukemia in a mammal in need thereof, the method comprising measuring the expression or activity of a FOXO transcription factor, and comparing the measured expression or activity of the FOXO transcription factor to a standard, wherein if the level of FOXO expression or activity is below the level of the standard, one proceeds to administer an effective amount of an agent that inhibits the expression or activity of a JNK protein kinase in a mammal, or rapamycin or a combination thereof, whereby leukemia is treated in the mammal.

In one embodiment, in practicing any one of aspects of the described methods, after a subject (e.g., human) has been diagnosed with leukemia but before any treatment, the subject's bone marrow progenitor cells or white blood cells are assessed to determine the level of FOXO and compared with a standard level of FOXO. If the FOXO level is below the standard described herein, e.g. 5% below the standard or below the standard by two standard deviations (SD) from the population mean or average, then the prescribed treatment comprises inhibiting the activity of the JNK pathway.

In another embodiment of this aspect and all other aspects described herein, the method of treating leukemia or inhibiting leukemia initiating cells (LICs) in a mammal further comprises measuring the expression or activity of AKT protein kinase, and comparing the measured expression or activity of the AKT protein kinase to a standard, wherein if the level of AKT protein kinase expression or activity is above the level of the standard, one proceeds to administer an effective amount of an agent that inhibits the expression or activity of the JNK protein kinase in a mammal and/or an effective amount of rapamycin.

In one embodiment of any aspects of the methods described herein, the subject's bone marrow progenitor cells or white blood cells are assessed to determine the expression or activity of AKT protein kinase (e.g., the phosphorylated AKT or other phosphorylated signaling molecules in the AKT signaling pathway known in the art), and comparing the measured the expression or activity of the AKT protein kinase to a standard, wherein if the level of AKT protein kinase expression or activity is above the level of the standard, one proceeds to administer an effective amount of an agent that inhibits the expression or activity of the JNK protein kinase in a mammal and an effective amount of rapamycin.

In one embodiment, in practicing any one of aspects of the described methods, the standard/reference level of the expression or activity of the AKT protein kinase is that normally found in a population of bone marrow progenitor cells (e.g., mononuclear bone marrow (MNBC) cells) from a healthy subject who do not have leukemia or has not been diagnosed with leukemia. In another embodiment of any one of aspects of the described methods, the standard/reference level of the expression or activity of the AKT protein kinase is that normally found in a population of white blood cells obtain from the peripheral circulation of a healthy subject who do not have leukemia or has not been diagnosed with leukemia. It is also contemplated that the standard/reference level of the expression or activity of the AKT protein kinase is the average of the standards or reference levels of the expression or activity of the AKT protein kinase obtain from a population of healthy subjects who do not have leukemia or have not been diagnosed with leukemia.

Also described herein in another aspect is a method of inhibiting leukemia initiating cells (LICs) in a mammal in need thereof, the method comprising administering an effective amount of an agent that inhibits the activity or expression of a FOXO transcription factor in the mammal, wherein the mammal has been diagnosed with leukemia and whereby the LICs are inhibited in the mammal.

In another aspect, embodied herein is a method of inhibiting leukemia initiating cells (LICs) in a mammal in need thereof, the method comprising administering an effective amount of any one of the compositions described herein to the mammal, wherein the mammal has been diagnosed with leukemia and whereby the LICs are inhibited in the mammal.

Also described herein in another aspect is a method of inhibiting LICs in a mammal in need thereof, the method comprising measuring the expression or activity of a FOXO transcription factor, and comparing the measured expression or activity of the FOXO transcription factor to a standard, wherein if the level of FOXO expression or activity is below the level of the standard, one proceeds to administer an effective amount of an agent that inhibits the expression or activity of a JNK protein kinase in a mammal, or rapamycin or a combination thereof, whereby LICs are inhibited in said mammal.

In another embodiment of this aspect and all other aspects described herein, the method of inhibiting LICs further comprises measuring the expression or activity of AKT protein kinase, and comparing the measured expression or activity of the AKT protein kinase to a standard, wherein if the level of AKT protein kinase expression or activity is above the level of the standard, one proceeds to administer an effective amount of an agent that inhibits the expression or activity of the JNK protein kinase in a mammal and an effective amount of rapamycin.

In one embodiment of any one aspect of the methods described herein, the mammal or subject is used interchangeably. In one embodiment of any one aspect of the methods described herein, the mammal is a human.

In one embodiment of any one aspect of the methods described herein, the method comprises selecting a mammal in need of treatment, e.g., after confirmatory diagnosis of leukemia.

### AKT and FOXO Signaling Pathway in Leukemia

AKT regulates numerous downstream modulators including inhibition of the FoxO family of transcription factors. This AKT/FOXO pathway is frequently upregulated in human cancers. AKT has oncogenic properties in human solid tumors and lymphomas, and FOXOs have tumor suppressive effects in similar malignancies.

Akt (c-Akt or PKB) is a proto-oncogene encoding a serine-threonine kinase (Testa, J. R. and Bellacosa, A., Leukemia Res., 1997, 21:1027-1031). It is the cellular homolog of the viral oncoprotein v-Akt, and is related to protein kinase-C(PKC) within the catalytic domain. However, c-Akt differs from the PKC family members by the presence of a pleckstrin homology (PH) domain at its N-terminus that is involved in the regulation of the activity of the enzyme by growth factors and intracellular signaling molecules. Various extracellular stimuli reportedly activate Akt through the phosphoinositide 3-kinase (PI 3-kinase) pathway (Datta, K. et al., J. Biol. Chem., 1996, 271:30835-30839; Franke, T. F., et al., Cell, 1995, 81:727-736; King, W. G. et al., Mol. Cell. Biol., 1997, 17:4406-4418). The lipid products of the PI 3-kinase reaction may activate Akt either by binding to the Akt pleckstrin homology domain (Franke, T. F. et al., 1997, Cell, 88:435:437), or by activating a protein kinase that phosphorylates Akt (Kohn, A. D., et al., J. Biol. Chem., 1996, 271:21920-21926; Stokoe et al., Science, 1997, 277:567-570). Activation of Akt reportedly inhibits apoptosis induced by growth factor withdrawal or irradiation in neural cells, fibroblasts, and lymphocytes (Franke, T. F., et al., Science, 1997, 275:665-668; Hemmings, Science, 1997, 275:628-630). It has been reported that Akt phosphorylates the pro-apoptotic protein Bad leading to Bad inactivation and cell survival (Datta, K., et al., Cell, 1997, 91:231-241; Peso, L., et al., Science, 1997, 278:687-689).

The AKT serine/threonine kinase is activated in a wide variety of human cancers (Altomare and Testa, 2005; Nicholson and Anderson, 2002). The prevalence of AKT activation in human cancer and its critical role in phosphoinositide 3-kinase (PI3K) signaling has motivated the development of several AKT inhibitors for treatment of advanced solid tumors and some hematological malignancies (Engelman, 2009; Garcia-Echeverria and Sellers, 2008; Liu et al., 2009; Martelli et al., 2009). AKT functions to promote tumorigenesis by phosphorylating and inactivating numerous substrates that antagonize cell growth and survival including PRAS40, GSK-3β, TSC2, BAD and the FOXO family transcription factors (Brunet et al., 1999; Cross et al., 1995; Datta et al., 1997; del Peso et al., 1997; Franke, 2008; Inoki et al., 2002; Kops and Burgering, 2000; Kops et al., 1999; Sancak et al., 2007; Tee et al., 2003; Wang et al., 2007). The kinase activity and substrate selectivity of AKT is controlled principally by two distinct phosphorylation events at threonine 308 (pAKT^{Thr308}) and serine 473 (pAKT^{Ser473}) via the actions of activated PI3K and mTORC2, respectively (Alessi et al., 1996; Alessi et al., 1997; Sarbassov et al., 2005; Stephens et al., 1998). Although pAKT^{Ser473} is dispensable for AKT-mediated phosphorylation of TSC2 and GSK-3β, pAKT^{Ser473} is required for phosphorylation and inactivation of the FOXOs (Guertin et al., 2006).

Direct mutations in components of the PI3K signaling pathway are rarely observed in AML, however, elevated AKT phosphorylation, which is indicative of AKT activation, has been observed in approximately 50% of human AML samples evaluated (Park et al., 2009). Detailed analysis of AKT modifications in human AML shows that pAKT^{Thr308} but not pAKT^{Ser473}, confers a poor prognosis in high-risk cytogenetic AML (Gallay et al., 2009). Also, constitutive pAKT^{Ser473} in patients with de novo AML correlates with a favorable response to chemotherapy, indicating that AKT activation in human AML is context-dependent (Tamburini et al., 2007).

The FOXO family of transcription factors, comprised of four highly related members FOXO1, FOXO3, FOXO4 and FOXO6, are direct downstream targets of AKT (Arden, 2006; Brunet et al., 1999; Burgering, 2008; Fu and Tindall, 2008; Kops and Burgering, 2000). While not wishing to be bound by theory, it has been postulated that in the absence of active AKT, FOXOs localize to the nucleus where they regulate the transcription of genes involved in cell cycle arrest, apoptosis and reactive oxygen species (ROS) detoxification. Upon AKT-mediated phosphorylation, FOXOs are exported to the cytoplasm and undergo proteasome-mediated degradation (Brunet et al., 1999; Carter and Brunet, 2007).

In some embodiments of the methods described herein, a mammal being treated with a FOXO inhibitor is also treated to increase or activate Akt activity. Akt activity can be increased by any method known to those of skill in the art including e.g., activation by small molecules or by delivery or expression of a constitutively active form of Akt (e.g., myrAkt, a myristolated form of Akt). Thus, Akt molecules as described herein include wild-type Akt molecules and constitutively-active Akt molecules. Akt can be activated by administration of e.g., Insulin-like Growth Factor-1 (IGF-1), Vascular Endothelial Growth Factor (VEGF) or constitutively-active Akt molecules. Given that Akt has been shown to play a role in several cancers, it is preferred that increasing Akt is performed in a cell-specific manner by e.g., delivering myrAkt under the control of a cell-specific promoter (e.g., a myeloid cell promoter). One of skill in the art can also make skilled decisions regarding the risks associated with treating a leukemia patient using an Akt activator. Such skilled decisions can be made by one of skill in the art on a case-by-case basis. For example, if an individual being treated with a FOXO inhibitor has a high risk for other cancers (e.g., a family history) one of skill in the art may opt not to combine the FOXO inhibitor with an Akt activator. Alternatively, if an individual is refractory to other treatments or has a poor leukemia prognosis while being treated with a FOXO inhibitor, one of skill in the art may opt to further treat that individual with an Akt activator.

Human and mouse Akt genes have been isolated and sequenced (Jones P F, et al., Proc Natl Acad Sci USA, 1991, 88(10):4171-5; Coffer, P. J. and Woodgett, J. R., Eur. J. Biochem., 1991, 201:475-481; Bellacosa, A., et al., Oncogene, 1993, 8:745-754). See also, GENBANK™ Accession No. M63167 (SEQ. ID. NO: 8), GENBANK™ Accession No. X61037 (SEQ. ID. NO: 9) for the human Akt cDNA and predicted amino acid sequences, respectively, and GENBANK™ Accession No. X65687 (SEQ. ID. NO: 10) for the mouse Akt cDNA. Other GENBANK™ Accession Nos. for human Akt isoforms include e.g., NM_{—}001014431.1 (SEQ. ID. NO: 23), NM_{—}001014432.1 (SEQ. ID. NO: 24), and NM_{—}005163.2 (SEQ. ID. NO: 25). The c-Akt protein contains, starting from its amino terminus, a src homology 2-like (SH2-like) domain (pleckstrin homology domain), and a kinase domain encoding a serine-threonine kinase with high degree of homology to members of the protein kinase C(PKC) family.

In some embodiments of the methods described herein, a mammal being treated with a FOXO inhibitor is also treated to decrease the activity or expression of a JNK protein kinase. It has been determined that approximately 60% of AMLs that display low FOXO activity display high expression levels of c-JUN, which is an oncogene. c-JUN is activated by JNK protein kinase and therefore, inhibition of JNK protein kinase leads to a decrease in c-JUN activity. Suitable JNK protein kinases and their inhibitors are known in the art and are described, for example in WO2008057252, WO2002010137, WO2006058007 and WO2002083648, U.S. Pat. Nos. 6,541,605; 6,897,231; 7,119,114; 7,351,729; 7,723,340; and U.S. patent application publication Nos. 2005/0107457, US 2005/0009876, 2003/0220330, 2004/0028660, 2004/0034084, 2005/0009876, 2003/0073732, and 2006/0258706, the entireties of which are incorporated herein by reference. The specific JNK inhibitor CC-401 (available from Celgene) is a potent inhibitor of all three forms of JNK (Ki of 25 to 50 nM) and has at least 40-fold selectivity for JNK compared with other related kinases. See Uehara et al. J. Hepatol. 2005 June; 42(6):850-9 and Uehara et al, Transplantation. 2004 Aug. 15; 78(3):324-32. JNK inhibitor CC-401 is 1-(5-(1H-1,2,4-triazol-5-yl)(1H-indazol-3-yl))-3-(2-piperidylethoxy)benzene and is disclosed in U.S. Pat. No. 6,897,231. Another JNK kinase inhibitor available from Celgene is CC-930 or 4-[9-(tetrahydro-furan-3-yl)-8-(2,4,6-trifluoro-phenylamino)-9H-purin-2-ylamino]-cyclohexan-1-ol, which is disclosed in U.S. patent publication No. US 2006/0287344. See also Wagner et al, Nature Reviews Cancer 9, 537-549 (2009) for information relating to clinical uses of CC-930 and CC-401.

In additional embodiments, for example where AKT activity or expression is high, a mammal being treated with a FOXO inhibitor and a JNK protein kinase inhibitor is also treated with rapamycin (RAPAMUNE® (sirolimus) oral solution and tablets, FDA approved in 1999).

### Leukemias

The methods and treatments described herein are applicable to essentially any leukemia or a related disorder. In one embodiment, the leukemia to be treated with the methods and compositions described herein is a myelogenous leukemia. Leukemia is clinically and pathologically subdivided into acute and chronic forms.

Acute leukemia is characterized by the rapid increase of immature blood cells, which impairs the ability of the bone marrow to produce healthy blood cells. Due to the rapid progression and accumulation of malignant cells, treatment should be applied immediately to prevent the spread of cancerous cells to other organs of the body. Acute forms of leukemia are the most common forms of leukemia in children.

Chronic leukemia is characterized by the build up of relatively mature, yet abnormal, white blood cells. Chronic leukemia typically takes months or years to progress. Abnormal cells are produced at a much higher rate than normal cells and result in the accumulation of abnormal white blood cells in the blood, bone marrow, spleen, thymus, lymph nodes, liver and other organs that sponsor the production of normal blood cells. Whereas acute leukemia must be treated immediately, chronic forms are sometimes monitored for some time before treatment is initiated to ensure maximum effectiveness of therapy. Chronic leukemia mostly occurs in adults, but can occur in any age group.

Further, leukemias are subdivided according to which blood cell is affected. For example, leukemias can be divided into lymphoblastic or lymphocytic leukemias and myeloid or myelogenous leukemias. In lymphoblastic or lymphocytic leukemias, a pre-lymphocyte cell is typically affected, which impairs the infection-fighting properties of cells derived from lymphocytes. Most lymphocytic leukemias involve B cells, a specific subtype of lymphocyte. In myeloid or myelogenous leukemias, red blood cell precursors are often affected as are some other types of white cells, and platelets. In one embodiment, the term “leukemia” as described herein also encompasses hairy cell leukemia and T-cell prolymphocytic leukemia, which are often considered to be outside of the above-described classification scheme. Acute lymphoblastic leukemia (ALL) is the most common leukemia in young children and includes, but is not limited to the following subtypes: precursor B acute lymphoblastic leukemia, precursor T acute lymphoblastic leukemia, Burkitt's leukemia, and acute biphenotypic leukemia. Chronic lymphocytic leukemia (CLL) most often affects adults over the age of 55 and also includes CLL subtypes, such as, B-cell prolymphocytic leukemia. Acute myelogenous leukemia (AML) occurs more commonly in adults than in children, and more commonly in men than women. Subtypes of AML include e.g., acute promyelocytic leukemia, acute myeloblastic leukemia, and acute megakaryoblastic leukemia. In acute promyelocytic leukemia, chromosomal changes in promyelocytes, cells that are at an early stage in the development into mature neutrophilsprevent binding and activity of vitamin A. Without vitamin A activity, normal cell maturation is disrupted, and abnormal promyelocytes accumulate. Chronic myelogenous leukemia (CML) occurs mainly in adults and includes, but is not limited to, chronic monocytic leukemia. In AML, the subtypes are based on: the specific types of cells involved in the leukemia; how mature the cancer cells are at the time of diagnosis; how different the cancer cells are from normal cells; and the number and location of specific changes within the cancer cells' DNA.

Leukemia patients often have reduced blood platelet levels, resulting in symptoms of abnormal clotting such as bruising easily, excessive bleeding or petechiae (pinprick bleeds). White blood cells, which are involved in fighting pathogens, can also be suppressed or dysfunctional in leukemia patients. Thus, some patients experience frequent infection, ranging from infected tonsils, sores in the mouth, or diarrhea to life-threatening pneumonia or opportunistic infections. Red blood cell deficiency can lead to anemia, which may cause dyspnea and pallor. Some patients experience other symptoms. These symptoms might include feeling sick, such as having fevers, chills, night sweats and other flu-like symptoms, or feeling fatigued. Some patients experience nausea or a feeling of fullness due to an enlarged liver and spleen; this can result in unintentional weight loss. If the leukemic cells invade the central nervous system, neurological symptoms can occur.

In one embodiment of the methods and compositions described herein, the targeted leukemia is a myelogenous leukemia. In another embodiment of the methods and compositions described herein, the leukemia is acute myelogenous leukemia.

In one embodiment, the targeted leukemia is not a hairy cell leukemia or T-cell prolymphocytic leukemia. In another embodiment, the targeted leukemia is not a lymphoblastic leukemia (e.g., ALL, CLL, or a sub-type thereof).

### Diagnosis of Leukemia

In some embodiments of the methods or aspects described herein, the subject is first diagnosed as having a leukemia disease or disorder prior to administering a FOXO inhibitor according to the methods described herein. In some embodiments, the subject is first diagnosed as being at risk of developing leukemia or disorder prior to administering a FOXO inhibitor.

Leukemia can be diagnosed by any method known in the art. Typically, a complete blood count (CBC) test is initially performed. A CBC counts the number of white blood cells, red blood cells, and platelets in a blood sample. A sample of blood with high numbers of white blood cells and low levels of red blood cells or platelets can indicate leukemia, and abnormal liver and kidney function tests will indicate if the leukemia has affected those organs. Flow cytometry can also be used for a more precise diagnosis, for example, by using mature myeloid markers such as CD11b and Gr-1 to determine cell type, cell number, and/or cell morphology.

For AML, there would be a drop in red blood cells, platelets, and normal white blood cells. These symptoms include fatigue, shortness of breath, easy bruising and bleeding, and increased risk of infection. The symptoms of are caused by replacement of normal bone marrow with leukemic cells, which are mainly immature abnormal white blood cells that accumulate in the bone marrow and interfere with the production of normal blood cells.

In one embodiment, a bone marrow biopsy is used to assist in diagnosis of leukemia. A bone marrow biopsy sample can include bone marrow tissue or a mixture of bone marrow and bone.

In another embodiment, cytogenetics is used to examine the chromosomes in individual cells. Cytogenetic testing uses a sample taken from a blood draw, a bone marrow or lymph node biopsy. The sample's chromosomes are microscopically examined for abnormalities that indicate damage to the cells' DNA and to support a diagnosis of leukemia.

In another embodiment, a spinal tap can be used in the diagnosis of leukemia. Typically, a sample of cerebrospinal fluid is taken from the lower back (the lumbar area). The fluid sample is then checked for leukemia cells and other abnormalities.

MR1s (Magnetic Resonance Imaging), CT (Computerized Axial Tomography) scans, and x-rays are imaging techniques that can also be used to support a diagnosis of leukemia.

Following a positive diagnosis for leukemia, blood chemistry tests can be used to determine the efficacy of chemotherapy or a FOXO inhibitor on the patient.

### FOXO Inhibitors (General)

Essentially any agent that inhibits FOXO activity, expression or nuclear translocation as those terms are defined herein can be used with the methods described herein. It is preferred, however, that an inhibitor of FOXO activity is specific, or substantially specific, for FOXO activity inhibition. Some non-limiting examples of agents include small molecules, RNA interference molecules, aptamers, ligands, peptides, nucleic acids, or a combination thereof. In addition, expression of a dominant negative mutant of a FOXO polypeptide can also be used to inhibit FOXO activity. Competitive mutants and/or competitive peptides of a FOXO polypeptide are also contemplated for use herein for inhibiting FOXO activity. Inhibitors of FOXO activities can be screened for efficacy by measuring FOXOs activity in the presence and absence of the inhibitor, using for example, a commercial kit such as the dual-luciferase assay by SABIOSCIENCES™ (a QIAGEN® company) or any other method known in the art or described herein. In one embodiment, a FOXO inhibitor interacts directly with a FOXO transcription factor to reduce its signaling activity. In another embodiment, a FOXO inhibitor causes phosphorylation of a FOXO transcription factor that leads to translocation of the factor from the nucleus leading to a decrease in FOXO activity. Inhibition of FOXO activity can be determined by measuring expression of one or more FOXO target genes.

### Small Molecule Inhibitors

As used herein, the term “small molecule” refers to a chemical agent including, but not limited to, peptides, peptidomimetics, amino acids, amino acid analogs, polynucleotides, polynucleotide analogs, nucleotides, nucleotide analogs, organic or inorganic compounds including heteroorganic and organometallic compounds having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Small molecule inhibitors of FOXO activity can be identified from within a small molecule library, which can be obtained from commercial sources such as AMRI (Albany, N.Y.), ASISCHEM Inc. (Cambridge, Mass.), TIMTEC (Newark, Del.), among others, or from libraries as known in the art. In one embodiment, a FOXO activity inhibitor can be an indirect inhibitor of FOXO activity, for example, an AKT activator increases AKT phosphorylation of FOXO resulting in an indirect decrease in FOXO activity. One of skill in the art can screen a small molecule library, such as those described above, for compounds having AKT activating activity.

### RNA Interference

RNA interference agents can be used with the methods described herein, to inhibit the expression and/or activity of a FOXO polypeptide. “RNA interference (RNAi)” is an evolutionarily conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002), herein incorporated by reference in its entirety), thereby inhibiting expression of the target gene. As used herein, “inhibition of target gene expression” includes any decrease in expression or protein activity or level of the target gene or protein encoded by the target gene as compared to a situation wherein no RNA interference has been induced. The decrease can be of at least 20%, 30%, 40%, 50%, 60%, 70%, 80%, 90%, 95% or 99% or more as compared to the expression of a target gene or the activity or level of the protein encoded by a target gene which has not been targeted by an RNA interfering agent. RNA interfering agents contemplated for use with the methods described herein include, but are not limited to, siRNA, shRNA, miRNA, and dsRNAi. The target gene or sequence of the RNA interfering agent can be a cellular gene or genomic sequence. An siRNA can be substantially homologous to the target gene or genomic sequence, or a fragment thereof. As used in this context, the term “homologous” is defined as being substantially identical, sufficiently complementary, or similar to the target mRNA or a fragment thereof, to effect RNA interference of the target. Preferably, the siRNA is identical in sequence to its target and targets only one sequence, i.e., it is specific. Each of the RNA interfering agents, such as siRNAs, can be screened for potential off-target effects by, for example, expression profiling. Such methods are known to one skilled in the art and are described, for example, in Jackson et al., Nature Biotechnology 6:635-637 (2003), herein incorporated by reference in its entirety. It is well within the ability of one skilled in the art to design and test for siRNAs that are useful for inhibiting FOXO expression and/or activity. There are known computer software products for predicting potential siRNA sequences using an inputted target mRNA sequence. For example, RNAstructure 4.4 from Michael and David Mathews, RNAi Central (Cold Spring Harbor Laboratory), siDIRECT (University of Tokyo, Japan), siRNA Selector (Wistar Institute) and siRNA Target Finder (AMBION®). It is important to note that double-stranded siRNA or shRNA molecules that are cleaved by Dicer in the cell can be up to 100 times more potent than a 21-mer siRNA or shRNA molecule supplied exogenously (Kim, D H., et al (2005) Nature Biotechnology 23(2):222-226). Thus, an RNAi molecule can be designed to be more effective by providing a sequence for Dicer cleavage. Methods for effective siRNA design for use in vivo can be found in U.S. Pat. No. 7,427,605, which is herein incorporated by reference in its entirety. Commercially available RNA interference molecules that target FOXO can be obtained from e.g., SANTA CRUZ BIOTECHNOLOGY® Inc. (Santa Cruz, Calif.), CELL SIGNALING TECHNOLOGY® (Danvers, Mass.), SIGMA ALDRICH® (St. Louis, Mo.), and THERMO SCIENTIFIC DHARMACON®, among others.

### In Vivo Delivery of RNA Interference (RNAi) Molecules

In general, any method of delivering a nucleic acid molecule can be adapted for use with an RNAi interference molecule (see e.g., Akhtar S, and Julian R L. (1992) Trends Cell. Biol. 2(5):139-144; WO94/02595, which are incorporated herein by reference in their entirety). However, there are three important factors for successfully delivering an RNAi molecule in vivo: (a) biological stability of the RNAi molecule, (2) preventing non-specific effects, and (3) accumulation of the RNAi molecule in the target tissue. The non-specific effects of an RNAi molecule can be minimized by local administration by e.g., direct injection into a target tissue including, for example, into the bone marrow, spleen or peripheral circulation system. Local administration of an RNAi molecule to a treatment site limits the exposure of the e.g., siRNA to systemic tissues and permits a lower dose of the RNAi molecule to be administered.

For the treatment of leukemia, the target tissue is the bone marrow. Thus, while leukemias do not generally provide a solid tumor mass amenable to direct delivery of agents such as RNA interference agents, direct delivery to or targeting of the bone marrow can be of use in the treatment of leukemias. In this regard, see, for example, WO2006/132388 (“Bone marrow-directing drug delivery materials and their applications”), which is incorporated herein by reference in its entirety. For administering an RNAi molecule systemically for the treatment of a disease, the RNAi molecule can be either be modified or alternatively delivered using a drug delivery system; both methods act to prevent the rapid degradation of the RNAi molecule by endo- and exo-nucleases in vivo. Modification of the RNAi molecule or the pharmaceutical carrier can also permit targeting of the RNAi molecule to the target tissue and avoid undesirable off-target effects. RNA interference molecules can be modified by chemical conjugation to lipophilic groups such as cholesterol to enhance cellular uptake and prevent degradation. For example, an siRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an RNAi molecule to an aptamer has been shown to inhibit tumor growth and mediate tumor regression in a mouse model of prostate cancer (McNamara, J O. et al (2006) Nat. Biotechnol. 24:1005-1015). In an alternative embodiment, the RNAi molecules can be delivered using drug delivery systems such as e.g., a nanoparticle, a dendrimer, a polymer, liposomes, or a cationic delivery system. The use of particulates for delivery to bone marrow is described, for example, in S. Moghimi “Exploiting bone marrow microvascular structure for drug delivery and future therapies.” Advanced Drug Delivery Rev. 17:61-73, 1995 and references cited therein, each of which are incorporated herein by reference in their entirety. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2):107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic administration of RNAi include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an RNAi molecule forms a complex with cyclodextrin for systemic administration. Methods for administration and pharmaceutical compositions of RNAi molecules and cyclodextrins can be found in U.S. Pat. No. 7,427,605, which is herein incorporated by reference in its entirety.

### Standards

In one embodiment of all aspects of the methods described herein, the expression or activity level of a FOXO transcription factor, JNK or AKT is compared to a standard or a reference standard. A standard can permit one of skill in the art to determine the amount of a FOXO, JNK or AKT or the relative increase/decrease of FOXO, JNK or AKT in a biological sample. A standard serves as a reference level for comparison, such that samples can be normalized to an appropriate standard in order to infer the presence, absence or extent of a leukemia in an individual or the response of an individual to treatment with a FOXO inhibitor. In one embodiment, a standard is obtained from the same individual as that being tested, at an earlier time point (i.e., before initiation of treatment with a FOXO inhibitor and/or a JNK protein kinase). Thus, a sample obtained from a patient is compared to a previously obtained sample, which acts as a reference. This type of standard is generally the most accurate for diagnostic, prognostic and efficacy monitoring purposes, since a majority of other markers will remain relatively similar from sample to sample in one individual. The standard should ideally be obtained prior to the onset of treatment or can be obtained prior to diagnosis when leukemia is suspected. At these times the levels of a FOXO transcription factor and/or AKT is at a baseline level for that individual at the time of testing. However, a standard can be obtained from an individual after the onset of leukemia as it can still provide information about improvement of symptoms or regression of the disease following treatment. For example, in some cases an increase in the amount of FOXO activity in a biological sample from an individual having leukemia can detect an increase in the risk of developing leukemia or a failure of a treatment to slow disease progress, while a decrease in amount of FOXO activity in a biological sample from an individual can indicate a regression of the disease or a decrease in risk of developing leukemia, as well as the need to employ an additional therapy (e.g., a JNK protein kinase inhibitor).

A standard can also be obtained from another individual or a plurality of individuals, wherein a standard represents an average level of FOXO or AKT activity among a population of individuals with or without leukemia. Thus, the level of FOXO or AKT in a standard obtained in this manner is representative of an average level of this factor in the given population, such as a general population of individuals having leukemia. An individual sample is compared to this population standard by comparing levels of FOXO or AKT activity/expression/nuclear localization from a biological sample relative to the standard. Generally, a decrease in the amount of FOXO activity or expression will indicate a decreased risk of a leukemia such as acute myeloid leukemia, or a regression in leukemia disease activity, while an increase in FOXO activity/expression/nuclear translocation will indicate an increased risk of leukemia as well as a progression in leukemia.

It should be noted that there can be variability among individuals in a population, such that some individuals will have very high levels of FOXO or AKT, while other individuals have very low levels of FOXO or AKT. However, one skilled in the art can make logical inferences on an individual basis regarding the determination of risk as described herein. A standard or series of standards can also be synthesized. A known amount of a FOXO or AKT activity or expression (or a series of known amounts) can be prepared within the typical expression range for the FOXO or AKT that is observed in a general leukemia population. This method has an advantage of being able to compare the extent of disease in two individuals in a mixed population. This method can also be useful for individuals who lack a prior sample to act as a standard or for routine screening of the general public. This type of method can also allow standardized tests to be performed among several clinics, institutions, or countries etc. A standard used in this manner can provide information about an individual's risk of developing leukemia in a manner similar to the cardiovascular risk that is assessed using routine monitoring of cholesterol and C-reactive protein in a blood sample.

### Dosage and Administration

In one aspect, the methods described herein provide a method for treating leukemia in a subject. In one aspect, the methods described herein provide a method for inhibiting leukemia initiating (LIC) cells in a subject. In one embodiment of all aspects of the methods described, the treatment comprises inhibiting leukemia initiating cells. In one embodiment of all aspects of the methods described, the treatment can be in vivo, i.e., in a subject or ex vivo. For example, bone marrow can be extracted from the subject diagnosed with leukemia. The bone marrow containing LIC can be treated as described herein and then transplanted back to the same subject. In one embodiment of all aspects of the methods described, the method comprises selecting a subject who has been diagnosed with leukemia for the treatment or inhibition. In one embodiment, the subject can be a mammal. In another embodiment, the mammal can be a human, although the approach is effective with respect to all mammals. The method comprises administering to the subject an effective amount of a pharmaceutical composition comprising an agent that inhibits FOXO activity, expression or translocation and/or an agent that inhibits JNK protein kinase activity and expression in a pharmaceutically acceptable carrier. The dosage range for the agent depends upon the potency, and includes amounts large enough to produce the desired effect, e.g., a reduction in activity of leukemia initiating cells. The dosage should not be so large as to cause unacceptable adverse side effects. Generally, the dosage will vary with the type of inhibitor used (e.g., small molecule, siRNA, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL. Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose. A therapeutically effective amount is an amount of an agent that is sufficient to produce a statistically significant, measurable change in a symptom of leukemia (see “Efficacy Measurement” below). Such effective amounts can be gauged in clinical trials as well as animal studies for a given FOXO or JNK protein kinase inhibitor, or an AKT activator. Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle. The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, a FOXO inhibitor, JNK inhibitor or AKT activator can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to a FOXO inhibitor, JNK inhibitor or AKT activator permits the agent attached to accumulate additively at the desired target site. For example, targeting specific cell types based on the expressed cell surface molecules on the target cell. Antibody-based or non-antibody-based targeting moieties can be employed to deliver a ligand or the inhibitor to a target site. Preferably, a natural binding agent for a leukemia associated antigen is used for this purpose. Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated. An agent can also be adapted for catheter-based delivery systems microencapsulated PEG liposomes, or nanobeads for delivery using direct mechanical intervention with or without adjunctive techniques such as ultrasound. In some embodiments, an inhibitor can be combined with one or more agents such as chemotherapeutic agents, for the treatment of a leukemia.

### Pharmaceutical Compositions

The present invention involves therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Specifically contemplated pharmaceutical compositions are active RNAi ingredients in a preparation for delivery as described herein above, or in references cited and incorporated herein in that section. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

### Efficacy Measurement

The efficacy of a given treatment for leukemia can be determined by the skilled clinician. However, a treatment is considered “effective treatment,” as the term is used herein, if any one or all of the signs or symptoms of, as but one example, activity of leukemia initiating cells is altered in a beneficial manner, other clinically accepted symptoms or markers of disease are improved, or even ameliorated, e.g., by at least 10% following treatment with an agent or agents as described herein (e.g., one or more of a FOXO inhibitor, JNK inhibitor or AKT activator). In one embodiment, efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of leukemic cells; or (2) relieving the disease, e.g., causing regression of symptoms, reducing the number of leukemia initiating cells; and (3) preventing or reducing the likelihood of the development of a leukemia). An effective amount for the treatment of a disease means that amount which, when administered to a mammal in need thereof, is sufficient to result in effective treatment as that term is defined herein, for that disease. Efficacy of an agent can be determined by assessing physical indicators of, for example leukemia, such as e.g., high white blood cell count, impaired immune response to infection, presence of leukemia initiating cells, etc.

The precise dose to be employed in the formulation of the agent will also depend on the route of administration, and the seriousness of the disease or disorder, and should be decided according to the judgment of the practitioner and each patient's circumstances. Routes of administration include, but are not limited to, intradermal, intramuscular, intraperitoneal, intravenous, subcutaneous, intranasal, epidural, and oral routes. The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Effective doses for each administrative route may be extrapolated from dose-response curves derived from in vitro or animal model test systems.

Efficacy testing can be performed during the course of treatment using the methods described herein. Measurements of the degree of severity of a number of symptoms associated with a particular ailment are noted prior to the start of a treatment and then at later specific time period after the start of the treatment. Leukemia is initiated in the bone marrow and can spread to other organs before it is detected, thus traditional staging of a tumor, performed routinely with other cancer types, is not useful in the staging of leukemia. Instead, physicians rely upon cytologic (cellular) classification systems to identify the type and subtype of leukemia. The prognosis or outcome of specific leukemias, and also the likely response to treatment can be determined using such cell classification systems. In one embodiment, the classification method for acute leukemia is the French-American-British (FAB) system. According to FAB classification, acute leukemia is divided into eight subtypes of acute myelogenous leukemia (AML) and three subtypes of acute lymphocytic leukemia (ALL). In another embodiment, the classification method for acute leukemia is the World Health Organization system. See Vardiman et al. (2002) Blood 100; 7 pp. 2292-2302. One of skill in the art is aware of such methods for determining disease severity in a variety of different leukemias and can easily diagnose the severity of the leukemia based on such a classification scheme.

The skilled artisan will appreciate that certain factors can influence the dosage and timing required to effectively treat a subject, including but not limited to the severity of the disease or disorder, previous treatments, the general health and/or age of the subject, and other diseases present. The dose levels can also depend on the particular inhibitor used, the severity of the symptoms and the susceptibility of the subject to side effects. Moreover, treatment of a subject with a therapeutically effective dose can include a single treatment or a series of treatments. Estimates of effective dosages and in vivo half-lives for various inhibitors encompassed by the invention can be made using conventional methodologies or on the basis of in vivo testing using an appropriate animal model, as known in the art, or as described herein. Preferred dosages for a given inhibitor are readily determinable by those of skill in the art by a variety of means.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention can be defined in any of the following numbered paragraphs:
[1] An agent that inhibits the activity or expression of a FOXO transcription factor for use in the treatment of leukemia in a mammal in need thereof.
[2] An agent that inhibits the activity or expression of a FOXO transcription factor for use in inhibiting leukemia initiating cells in a mammal in need thereof.
[3] The use of paragraph 1 or 2, wherein the FOXO transcription factor is FOXO 1, FOXO 3, FOXO4 or FOXO6.
[4] The use of paragraph 1 or 2, wherein the agent is selected from the group consisting of a small molecule, an oligonucleotide or an RNAi agent.
[5] The use of any one of paragraphs 1-4, wherein the agent inhibits the activity of the FOXO transcription factor in the mammal.
[6] The use of any one of paragraphs 1-4, wherein the agent inhibits the expression of the FOXO transcription factor in the mammal.
[7] The use of any one of paragraphs 1-4, wherein the agent increases translocation of the FOXO transcription factor out of the nucleus.
[8] The use of any one of paragraphs 1-4, and 6, wherein the agent is an RNAi agent that inhibits the expression of the FOXO transcription factor in the mammal.
[9] The use of paragraph 8, wherein the RNAi agent comprises a double-stranded ribonucleic acid (dsRNA) wherein the dsRNA comprises a sense strand and an antisense strand, the antisense strand comprising a region of complementarity to and having at least 15 contiguous nucleotides differing by no more than 3 nucleotides from a molecule selected from the group consisting of SEQ ID NOs:11-22.
[10] The use of paragraph 9, wherein the RNAi agent comprises the nucleotide sequence of one of SEQ ID NOs: 11-14.
[11] The use of any one of paragraphs 1-4, 6, and 8-9, wherein the agent is a vector comprising a nucleic acid sequence that is at least 90% identical to one of SEQ. ID. Nos. 11-22.
[12] The use of paragraph 11, wherein the vector is a viral vector.
[13] The use of any one of paragraphs 1-12, wherein the mammal is a human.
[14] The use of any one of paragraphs 1-13, further comprising measurement of the expression or activity of a FOXO transcription factor prior to use of the agent or medicament comprising the agent, and comparing the measured expression or activity of said FOXO transcription factor to a standard, wherein if the level of FOXO expression or activity is above the level of said standard, one proceeds with use of the agent or medicament comprising the agent.
[15] The use of any one of paragraphs 1-14, further comprising the use of an agent that increases the expression or activity of the AKT protein kinase in the mammal.
[16] The use of paragraph 15, wherein the agent that increases the expression or activity of the AKT protein kinase is selected from the group consisting of a small molecule, an aptamer, a peptide, a protein, and an oligonucleotide.
[17] The use of paragraph 15 or 16, wherein the agent that increases the expression or activity of the AKT protein kinase is a vector comprising a sequence encoding an AKT protein kinase.
[18] The use of paragraph 17, wherein the vector is an expression vector comprising a nucleic acid sequence that is at least 95% sequence identical to SEQ. ID. No. 23.
[19] The use of paragraph 16, wherein the agent is a small molecule activator of AKT activity and is selected from the group consisting of rapamycin, CCl-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, insulin-like growth factor-1, platelet derived growth factor and granulocyte colony-stimulating factor (G-CSF).
[20] The use of any one of paragraphs 1-19, further comprising administering an effective amount of an agent that inhibits the expression or activity of a JNK protein kinase in the mammal.
[21] The use of paragraph 20, wherein the agent that inhibits activity of JNK protein kinase is 4-[9-(tetrahydro-furan-3-yl)-8-(2,4,6-trifluoro-phenylamino)-9H-purin-2-ylamino]-cyclohexan-1-ol (JNK 930/CC-930) or 1-(5-(1H-1,2,4-triazol-5-yl)(1H-indazol-3-yl))-3-(2-piperidylethoxy)benzene (JNK 401/CC-401).
[22] The use of any one of paragraphs 1-21, further comprising administering an effective amount of rapamycin.
[23] The use of any one of paragraphs 1-22, wherein the leukemia is acute myelogenous leukemia.
[24] A method of treating leukemia in a mammal in need thereof, the method comprising administering an effective amount of an agent that inhibits the activity or expression of a FOXO transcription factor in the mammal, whereby leukemia is treated in said mammal.
[25] A method of inhibiting leukemia initiating cells (LICs) in a mammal in need thereof, the method comprising administering an effective amount of an agent that inhibits the activity or expression of a FOXO transcription factor in the mammal, wherein said mammal has been diagnosed with leukemia and whereby the LICs are inhibited in said mammal.
[26] The method of paragraph 24 or 25, wherein the FOXO transcription factor is FOXO 1, FOXO3, FOXO4 or FOXO6.
[27] The method of paragraph 24, 25 or 26, wherein the agent is selected from the group consisting of a small molecule, an oligonucleotide or an RNAi agent.
[28] The method of any one of paragraphs 24-27, wherein the agent inhibits the activity of the FOXO transcription factor in the mammal.
[29] The method of any one of paragraphs 24-27, wherein the agent inhibits the expression of the FOXO transcription factor in the mammal.
[30] The method of any one of paragraphs 24-27, wherein the agent increases translocation of the FOXO transcription factor out of the nucleus.
[31] The method of any one of paragraphs 24-27 and 29, wherein the agent is an RNAi agent that inhibits the expression of the FOXO transcription factor in the mammal.
[32] The method of paragraph 31, wherein the RNAi agent comprises a double-stranded ribonucleic acid (dsRNA) wherein the dsRNA comprises a sense strand and an antisense strand, the antisense strand comprising a region of complementarity to and having at least 15 contiguous nucleotides differing by no more than 3 nucleotides from a molecule selected from the group consisting of SEQ ID NOs:11-22.
[33] The method of paragraph 32, wherein the RNAi agent comprises the nucleotide sequence of one of SEQ ID NOs: 11-14.
[34] The method of any one of paragraphs 24-27, 29 and 31-33, wherein the agent is a vector comprising a nucleic acid sequence that is at least 90% identical to one of SEQ. ID. Nos. 11-22.
[35] The method of paragraph 34, wherein the vector is a viral vector.
[36] The method of any one of paragraphs 24-35, wherein the mammal is a human.
[37] The method of any one of paragraphs 24-36, further comprising the steps, before said administering step, of measuring the expression or activity of a FOXO transcription factor, and comparing the measured expression or activity of said FOXO transcription factor to a standard, wherein if the level of FOXO expression or activity is above the level of said standard, one proceeds to said administering step.
[38] The method of any one of paragraphs 24-37, further comprising administering an effective amount of an agent that increases the expression or activity of the AKT protein kinase in the mammal.
[39] The method of paragraph 38, wherein the agent that increases the expression or activity of the AKT protein kinase is selected from the group consisting of a small molecule, an aptamer, a peptide, a protein, and an oligonucleotide.
[40] The method of paragraph 38 or 39, wherein the agent that increases the expression or activity of the AKT protein kinase is a vector comprising a sequence encoding an AKT protein kinase.
[41] The method of paragraph 40, wherein the vector is an expression vector comprising a nucleic acid sequence that is at least 95% identical to SEQ. ID. No. 23.
[42] The method of paragraph 38 or 39, wherein the agent is a small molecule activator of AKT activity and is selected from the group consisting of rapamycin, CCl-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, insulin-like growth factor-1, platelet derived growth factor and granulocyte colony-stimulating factor (G-CSF).
[43] The method of any one of paragraphs 24-44 further comprising administering an effective amount of an agent that inhibits the expression or activity of a JNK protein kinase in the mammal.
[44] The method of paragraph 43, wherein the agent that inhibits activity of JNK protein kinase is 4-[9-(tetrahydro-furan-3-yl)-8-(2,4,6-trifluoro-phenylamino)-9H-purin-2-ylamino]-cyclohexan-1-ol (JNK 930/CC-930) or 1-(5-(1H-1,2,4-triazol-5-yl)(1H-indazol-3-yl))-3-(2-piperidylethoxy)benzene (JNK 401/CC-401).
[45] The method of any one of paragraphs 24-44, further comprising administering an effective amount of rapamycin.
[46] The method of any one of paragraphs 24-45, wherein the leukemia is acute myelogenous leukemia.
[47] A method of treating leukemia in a mammal in need thereof, the method comprising measuring the expression or activity of a FOXO transcription factor, and comparing the measured expression or activity of said FOXO transcription factor to a standard, wherein if the level of FOXO expression or activity is below the level of said standard, one proceeds to administer an effective amount of an agent that inhibits the expression or activity of a JNK protein kinase in a mammal, or rapamycin or a combination thereof, whereby leukemia is treated in said mammal.
[48] The method of paragraph 47, further comprising measuring the expression or activity of AKT protein kinase, and comparing the measured expression or activity of said AKT protein kinase to a standard, wherein if the level of AKT protein kinase expression or activity is above the level of said standard, one proceeds to administer an effective amount of an agent that inhibits the expression or activity of the JNK protein kinase in a mammal and an effective amount of rapamycin.
[49] A method of inhibiting leukemia initiating cells (LICs) in a mammal in need thereof, the method comprising measuring the expression or activity of a FOXO transcription factor, and comparing the measured expression or activity of said FOXO transcription factor to a standard, wherein if the level of FOXO expression or activity is below the level of said standard, one proceeds to administer an effective amount of an agent that inhibits the expression or activity of a JNK protein kinase in a mammal, or rapamycin or a combination thereof, wherein said mammal has been diagnosed with leukemia and whereby the LICs are inhibited in said mammal.
[50] The method of paragraph 49, further comprising measuring the expression or activity of AKT protein kinase, and comparing the measured expression or activity of said AKT protein kinase to a standard, wherein if the level of AKT protein kinase expression or activity is above the level of said standard, one proceeds to administer an effective amount of an agent that inhibits the expression or activity of the JNK protein kinase in a mammal and an effective amount of rapamycin.
[51] The method of any one of paragraphs 47-50, wherein the agent that inhibits activity of JNK protein kinase is 4-[9-(tetrahydro-furan-3-yl)-8-(2,4,6-trifluoro-phenylamino)-9H-purin-2-ylamino]-cyclohexan-1-ol (JNK 930/CC-930) or 1-(5-(1H-1,2,4-triazol-5-yl) (1H-indazol-3-yl))-3-(2-piperidylethoxy)benzene (JNK 401/CC-401).
[52] The method of paragraph 48 or 50, wherein the agent that increases the expression or activity of the AKT protein kinase is selected from the group consisting of a small molecule, an aptamer, a peptide, a protein, and an oligonucleotide.
[53] The method of paragraph 52, wherein the agent that increases the expression or activity of the AKT protein kinase is a vector comprising a sequence encoding an AKT protein kinase.
[54] The method of paragraph 53, wherein the vector is an expression vector comprising a nucleic acid sequence that is at least 95% identical to SEQ. ID. No. 23.
[55] The method of any one of paragraphs 48, 50 and 52, wherein the agent is a small molecule activator of AKT activity and is selected from the group consisting of rapamycin, CCl-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, insulin-like growth factor-1, platelet derived growth factor and granulocyte colony-stimulating factor (G-CSF).
[56] The method of any one of paragraphs 47-55, wherein the leukemia is acute myelogenous leukemia.
[57] The method of any one of claims 47-55, wherein the FOXO transcription factor is FOXO 1, FOXO 3, FOXO 4 or FOXO 6.

This invention is further illustrated by the following examples, which should not be construed as limiting. The contents of all references cited throughout this application, as well as the figures and tables thereof are incorporated herein by reference in their entirety.

Those skilled in the art will recognize, or be able to ascertain using not more than routine experimentation, many equivalents to the specific embodiments of the invention described herein. Such equivalents are intended to be encompassed by the following claims.

### EXAMPLES

### Example 1

### Exemplary Materials and Methods

### Plasmid and Mouse Generation

The MSCV-MLL-AF9 construct was obtained from Dr. S. A. Armstrong (Children's Hospital of Boston). The MSCV-IRES-GFP-myrAKT construct was provided by Dr. K. Gritsman (Dana-Farber Cancer Institute) and Dr. Michael G. Kharas (Children's Hospital of Boston). The MSCV-puro-CreER plasmid was provided by Dr. D. Kalaitzidis (Children's Hospital of Boston). The expression plasmid carrying the LSL-hCD34 was provided by Dr. M. Milsom (Heidelberg University). The FoxO1/3/4^{floxed}; Mx1-Cre+ mice were generated previously (Paik et al., 2007; Tothova et al., 2007).

### AML Patient Samples

Bone marrow (BM) aspirates from patients with AML were collected under a protocol approved by the institutional review board (IRB) of Massachusetts General Hospital. Ficoll density gradient was then used to recover viable mononuclear cells from BM aspirates.

### MLL-AF9 Retroviral Bone Marrow Transplantation Assay

Both FoxO1/3/4^{floxed}; Mx1-Cre+ and FoxO1/3/4^{floxed}; Mx1-Cre− mice were administered 150 mg/kg 5-fluorouracil (5-FU, SIGMA ALDRICH® ALDRICH®). Six days following 5-FU treatment mice were euthanized and mononuclear bone marrow (BM) cells recovered were subjected to retroviral transduction with recombinant MLL-AF9 expressing retroviruses as follows:
Day 1: Seed 3−4×10⁶ 293FT or 293TL cells/10 cm dish.
Day 2: Approximately 60-80% confluency: Transfect cells with DNA:FUGENE® Mix as follows.
   a. Add 18 ul of FUGENE® to 282 ul of plain DMEM (No serum or P/S). Tap tube to mix and incubate 5 minutes at room temperature.
   b. Add 3 ug of lentiviral backbone (i.e. pKLO shRNA), 3 ug of each packaging plasmid (i.e. 3 ug of VSV-G+3 ug of pCMVΔ8.2 OR 3 ug of MD+3 ug of pSPAX) to FUGENE®:DMEM mix. Incubate 15-30 minutes at room temperature.
   c. Add DNA:FUGENE®:DMEM mix to 5 ml of DMEM/10% FBS (No P/S).
   d. Aspirate media from 293FT cells and replenish with 5.5 ml DNA:FUGENE®:DMEM:Full Serum mix. Incubate overnight in a 37° C. incubator.
Day 3: Aspirate transfection mixture from each 10 cm plate and replenish with 3.5 ml DMEM/30% FBS. Incubate overnight in a 37° C. incubator and ensure the plates are on a flat surface and all cells are covered with a thin layer of media.
Day 4: Collect ˜3.5 ml viral supernatant and place in a 50 ml conical tube and store overnight in the dark at 4° C. (Viral Sup. No. 1). Replenish with 3.5 ml DMEM/30% FBS. Incubate overnight in a 37° C. incubator and ensure the plates are on a flat surface and all cells are covered with a thin layer of media.
Day 5: Collect ˜3.5 ml viral supernatant and combine with Viral Sup. No. 1 & 2 in a 50 ml conical tube. Centrifuge combined viral supernatant 5 minutes at 1200 rpm. Aliquot centrifuged virus into cryovials and store at −80° C. Infect 1×10⁶ suspension cells in a 6-well plate with a total volume of 2.0 ml which is composed of 500 ul of viral supernatant+8 ug/ml polybrene+1.5 ml of media.

After transduction, infected cells were transplanted into lethally irradiated (2 doses of 450rad gIR separated by 12 hours) F1 FVB/C57/Bl6 mice. Recipient mice developed leukemia within 70-80 days with a median survival of 77 days. Mononuclear BM cells recovered from leukemic mice were then transplanted into secondary sub-lethally irradiated recipients. Fourteen days following transplant, secondary recipients were administered intra-peritoneally three doses of saline or 12.5 mg/kg pI-pC (GE Healthcare Life Sciences) every 2 days. Mice were then monitored for external (i.e. moribund) and internal (white blood cell counts) signs of leukemia.

### Flow Cytometry and Antibodies

L-GMP and GMP populations were analyzed and sorted with a BD FACSARIA™ instrument (BD™ Biosciences). To analyze and isolate L-GMPs and GMPs, total BM cells were recovered from flushing the tibias and femurs of leukemic and healthy mice respectively with PBS supplemented with 2% FBS and 1% Penicillin/Streptomycin (F-PBS). Bone marrow mononuclear cells (BMMCs) were then stained with a lineage cocktail comprised of antibodies targeting CD3, CD4, CD8, CD19, B220, Gr-1, Ter119, and IL-7RαPE-Cy5 conjugates). Cells were also stained with antibodies targeting cKit-Ax750, Sca-1-APC or -Pacblue, FcγRII/III-PE-Cy7, and CD34-FITC. For Phosphoflow experiments, cells were stained with cKit-Ax750 and CD34-antibodies in combination with phospho-AKT^{Ser473}-PE, phospho-AKT^{Thr308}-APC or phospho-S6^{Ser235/236}-APC antibodies and analyzed on a FACSAria according to the following protocol:
1. Euthanize mice and recover tibias and femurs. Use a scapel to cut the ends of each bone off and use a syringe with a 271/2G needle (filled with PBS supplemented with 2% FBS and 1% P/S=F-PBS) flush into a 50 ml conical tube covered with a 70 uM nylon filter.
2. Lyse cells with RBC lysis buffer (BD™ Biosciences) for 5-10 minutes on ice. Wash with 10.0 ml F-PBS and centrifuge 5 min at 1500 rpm. Aspirate supernatant and wash one more time with 5.0 ml F-PB S. Centrifuge 5 min at 1500 rpm. Aspirate supernatant and resuspend cells in 400 ul of F-PBS containing lineage antibodies conjugated with PE-Cy5 at the dilutions tabulated above. Incubate on ice in the dark for 20 min. Wash cells with 4.0 ml F-PBS. Centrifuge 5 min at 1500 rpm. Repeat. Aspirate supernatant and resuspend cells in 400 ul of F-PBS.
3. Sort lineage low (10-15%) into IMDM supplemented with 2% FBS (F-IMDM). Rest cells in IMDM supplemented with 2% FBS for 1 hour at 37° C.
4. Treat cells with 10 ng/ml mSCF (or 1 ng/ml GM-CSF, 10 ng/ml IL-3 or 10 ng/ml IL-6) for 5 and 15 minutes and then immediately fix with 1.6% paraformaldehyde (PFA) for 10 minutes at 37° C.
5. Centrifuge 5 minutes at 1500 rpm. Aspirate supernatant and resuspend pellet in 1.0 ml of ice-cold 95% methanol while vortexing. Incubate on ice for 10 minutes. Transfer cells to a 1.5 ml eppendorf tube and centrifuge 3 min at 6000 rpm. Aspirate and wash by adding 1 mL of 0.50% BSA/0.02% NaN₃/PBS. Pellet cells (as above), aspirate. Add another 1 mL of 0.50% BSA/0.02% NaN₃/PBS. Pellet cells, aspirate.
6. Stain cells for 20 min in the dark on ice with cKit-Ax750 and CD34-FITC or Pacblue antibodies in combination with phospho-AKT^{Ser473}-PE, phospho-AKT^{Thr308}-APC or phospho-S6^{Ser235/236}-APC antibodies. NOTE: Alcohol permeabilization destroys the FcγRII/III epitope and therefore these specific antibodies were excluded (Kalaitzidis and Neel, 2008)). Wash by adding 1 mL of 0.50% BSA/0.02% NaN₃/PB S. Pellet cells (as above), aspirate. Resuspend cells in 300-400 ul and analyze on a FACSARIA™ instrument.

TABLE 1
Exemplary Phosphoflow Antibodies

| | | CLONE# | |
|---|---|---|---|
| | | OR | DILUTION |
| ANTIBODY | COMPANY | CATALOG# | FACTOR |
| CD3 | BIOLEGEND ® | 145-2c11 | 1:40 |
| CD4 | BD ™ Biosciences | RM4-5 | 1:100 |
| CD8 | BIOLEGEND ® | 53-6.7 | 1:100 |
| CD19 | BIOLEGEND ® | 6D5 | 1:100 |
| B220 | EBIOSCIENCE ® | RA3-6B2 | 1:100 |
| Gr-1 | EBIOSCIENCE ® | RB6-8C5 | 1:100 |
| Ter119 | BIOLEGEND ® | TER-119 | 1:400 |
| IL-7Ra | EBIOSCIENCE ® | A7R34 | 1:30 |
| CD11b | BIOLEGEND ® | | 1:100 |
| cKit | EBIOSCIENCE ® | 2B8 | 1:200 |
| Sca-1 | BIOLEGEND ® | D7 | 1:200 |
| CD34 | EBIOSCIENCE ® | RAM34 | 1:50 |
| FcgRII/III | EBIOSCIENCE ® | 93 | 1:200 |
| Phosphor-AKT^{Ser473}-PE | CELL SIGNALING | D9E | 1:25 |
| | TECHNOLOGY ® | | |
| | (CST) | | |
| phospho-AKT^{Thr308}-APC | CST | C31E5E | 1:25 |
| phospho-S6^{Ser235/236}-APC | CST | D57.2.2E | 1:100 |
| Human CD11b | BD ™ Biosciences | ICRF44 | 1:100 |
| Human CD34-APC | BD ™ Biosciences | 8G12 | 5 ul/1 × 10⁶ |
| | | | cells |
| Human Lineage cocktail | BD ™ Biosciences | 340546 | 5 ul/ 1 × 10⁶ |
| | | | cells |

Mature myeloid markers of murine MLL-AF9-expressing leukemia cells were assessed with mouse CD11b-APC and Gr-1-PE-Cy5 antibodies. Human AML, APL and CML cell lines were stained with human CD11b-APC antibodies. Primary AML patient BM cells were stained with human CD34-APC and Lineage cocktail-FITC.

### Cell Death Assays

Cells were stained with AnnexinV-PE and 7-AAD according to the manufacturer instructions (BD™ Biosciences) to assess levels of apoptosis. Where indicated, cell death was also evaluated with Trypan Blue staining (CELLGRO®).

### Phagocytosis Assay

Phagocytosis was assessed using a pHRODO™ BIOPARTICLES® Conjugates for Phagocytosis Kit (INVITROGEN™). Assays were performed according to the manufacturer's instructions.

### Indirect Immunofluorescence

Purified L-GMPs and GMPs were placed on microscope slides via cytospin (4 minutes at 450 rpm). Cells were then fixed with 1% PFA and permeabilized with 100% methanol. Processed cells were then subjected to staining with FOXO3 (75D8) antibodies (1:1000; CELL SIGNALING TECHNOLOGY®). FOXO3 stained cells were then incubated with an anti-rabbit FITC-conjugated secondary antibody (1:2000; SIGMA ALDRICH®). Cells were also stained with DAPI to visualize nuclei. Cells were then visualized under 100× magnification using a Nikon fluorescence microscope. The protocol is summarized as follows:
1. Euthanize mice and recover tibias and femurs. Use a scapel to cut the ends of each bone off and use a syringe with a 271/2G needle (filled with PBS supplemented with 2% FBS and 1% P/S═F-PBS) flush into a 50 ml conical tube covered with a 70 uM nylon filter.
2. Lyse cells with RBC lysis buffer (BD™ Biosciences) for 5-10 minutes on ice. Wash with 10.0 ml F-PBS and centrifuge 5 min at 1500 rpm. Aspirate supernatant and wash one more time with 5.0 ml F-PBS. Centrifuge 5 min at 1500 rpm. Aspirate supernatant and resuspend cells in 400 ul of F-PBS plus lineage antibodies conjugated with PE-Cy5, cKit-Ax750, Sca-1-APC, Fc2RII/III-PE-Cy7 and CD34-FITC with the appropriate dilutions tabulated above. Incubate on ice for 20 min in the dark. Wash cells with 4.0 ml F-PBS. Centrifuge 5 min at 1500 rpm. Repeat.
3. Sort cells using FACSARIA™ (BD™ Biosciences). Purified L-GMPs and GMPs were placed on microscope slides via cytospin (4 minutes at 450 rpm), Cells were then fixed pith 1% PFA for 10 minutes at room temperature,
4. Aspirate PFA and permeabilize with 100% methanol. Wash cells two times with BS-T, Block cells with 5% BSA at room temperature.
5. Incubate cells with FOXO3 (75D8) antibodies (1:1000; CELT, SIGNALING TECHNOLOGY®) overnight at 4° C.
6. Incubate FOXO3 stained cells with an anti-rabbit MC-conjugated secondary antibody (1:2000; SIGMA ALDRICH®). Stain cells with DAPI to visualize nuclei,
7. Visualize cells under 100× magnification using a Nikon fluorescence microscope.

### Cell Fractionation

To recover cytoplasm fractions, both murine and human AML cells were resuspended in hypotonic buffer (10 mM HEPES pH7.9, 10 mM KCl, 1.5 mM MgCl₂, 0.34M sucrose, 10% glycerol, 1 mM DTT, 0.4% NP-40) and placed on ice for 10 minutes. Nuclei and cytoplasm fractions were then separated by centrifugation for 4 min at 1,300×g. Cytoplsmic fractions (supernatant) were recovered and nuclei (pellet) were subsequently lysed with hypertonic buffer (20 mM HEPES, pH7.9, 25% Glycerol, 420 mM NaCl, 1.5 mM MgCl₂, 0.5 mM EDTA, 0.5 mM PMSF, 0.5 mM DTT) for 30 minutes on ice. The protocol is summarized as follows:
1. Resuspend cells in 2-3 pellet volumes of Buffer A (10 mM HEPES pH7.9, 10 mM KCl, 1.5 mM MgCl₂, 0.34M sucrose, 0.4% NP-40, 10% glycerol, 1 mM DTT and Protease Inhibitors). Smaller volumes generate more concentrated cytoplasmic fractions. Incubate cells on ice for 20 minutes.
2. Pellet nuclei by centrifugation (4 minutes at 1,300×g (−3500 rpm on a microcentrifuge), 4° C.). Recover supernatant (Cytoplamic fraction). Wash nuclei 1× with Buffer A and centrifuge 4 minutes at 1,300×g, 4° C.
3. Aspirate supernatant and resuspend the pellet in 1-2 pellet volumes of buffer C (20 mM HEPES, pH7.9, 25% Glycerol, 420 mM NaCl, 1.5 mM MgCl₂, 0.5 mM EDTA, 0.5 mM PMSF, 0.5 mM DTT). Incubate on ice for 30 minutes with vortexing for 10 sec every 5 minutes or spin at 60 rpm at 4° C.

Nuclear and cytoplamic fractions were then subjected to western blot with antibodies targeting FOXO3 (1:1000, clone 75D8, CELL SIGNALING TECHNOLOGY®), Tubulin (1:2500, CELL SIGNALING TECHNOLOGY®) and ORC2 (1:1000, SANTA CRUZ BIOTECHNOLOGY®) as follows:
1. Protein lysates were generated by counting cells with trypan blue staining and then lysing cells directly in 1×LDS buffer supplemented with 1× reducing agent (INVITROGEN™) at a ratio of 1×10⁶ cells per 100 ul of 1×LDS. Lysates were boiled 10 min and then centrifuged for 5 min at 14000 rpm.
2. Lysates were then loaded onto either 7% Tris-Acetate gels (INVITROGEN™) or 4-20% Tris-Glycine gels (INVITROGEN™) and run at 100V for approximately 90-150 min.
3. Gels were then transferred onto nitrocellulose membranes using the INVITROGEN™ wet gel transfer system (120 min at 30V).
4. Nitrocellulose membranes were then blocked in 5% BSA TBS-T for 1 hour at room temperature. Membranes were then dissected based on protein size markers and placed in the appropriate antibodies diluted in 1% BSA/TBS-T. Membranes were incubated overnight on a rocker at 4° C. Membranes were washed three times in 15-25 ml 1×TBS-T for 10 minutes each.
5. Membranes were incubated with HRP-conjugated anti-rabbit (or anti-mouse depending on the primary antibody) secondary antibody (Amersham, GE Healthcare Life Sciences) at a dilution of 1:2500 for 1 hour at room temperature. Membranes were washed three times in 15-25 ml 1×TBS-T for 10 minutes each.
6. Membranes were incubated with SUPERSIGNAL® West Pico ECL kit available from PIERCE TECHNOLOGIES®. Expose to film and develop in an X-OMAT developer.

TABLE 2
Exemplary Western Blot Antibodies

| | | CLONE# | |
|---|---|---|---|
| | | OR | DILUTION |
| ANTIBODY | COMPANY | CATALOG# | FACTOR |
| FOXO3 | CST | 9467 | 1:1000 |
| FOXO3 (75D8) | CST | 2497 | 1:1000 (W) |
| | | | 1:100 (IF) |
| Tubulin | CST | 9F3 | 1:2500 |
| ORC2 | SANTA CRUZ | H-300 | 1:1000 |
| | BIOTECHNOLOGY ® | | |
| Phospho-FOXO3 | CST | 9466 | 1:1000 |
| (Ser256) | | | |
| Phospho-ERK1/2 | CST | 197G2 | 1:1000 |
| (Thr202/Tyr204) | | | |
| Phospho-AKT | CST | 193H12 | 1:1000 |
| (Ser473) | | | |
| Phospho-AKT | CST | 244F9 | 1:1000 |
| (Thr308) | | | |
| Phopho-4E-BP1 | CST | 236B4 | 1:1000 |
| (Thr37/46) | | | |
| Phospho-S6 | CST | 2F9 | 1:1000 |
| (Ser235/236) | | | |
| Phospho-cJUN | CST | 54B3 | 1:1000 |
| (Ser63) | | | |
| cJUN | CST | 9162 | 1:1000 |
| Phospho-JNK | CST | 98F2 | 1:1000 |
| (Thr183/Tyr185) | | | |
| JNK | CST | 9252 | 1:1000 |

### Cell Morphology Staining

To examine morphological changes associated with myeloid differentiation, cells from respective conditions were centrifuged (4 minutes at 450 rpm) onto microscope slides. Cells were then permeabilized in 100% methanol for 4 minutes and subsequently stained with May-Grünwald dye (SIGMA ALDRICH®) for 10 minutes. Cells were then stained 1 minute with Giemsa dye, washed with distilled water and allowed to air dry. For cells stained with Wirght-Giemsa, cells were adhered to microscope slides as described above. Cells were permeabilized and stained in 100% Wright-Giemsa stain for 4 minutes. Cells were then stained for 10 minutes with 20% Wright-Giemsa/80% Cell wash buffer and subsequently washed with distilled water and allowed to air dry.

### Colony Assays

For murine myeloid colony assays, three replicates of 1000 cells were plated in 1.5 ml M3434 cytokine enriched methylcellulose according to manufacturer's instructions (STEMCELL™ Technologies). For human myeloid colony assays, three replicates of 10000 cells were plated in 1.5 ml H4034 cytokine enriched methylcellulose according to manufacturer's instructions (STEMCELL™ Technologies). For CAFC assays, murine leukemia cells were plated on OP9 stroma cell at frequency 0, 2, 5, 10, 20, 40, 80, 160 and 320 cells per well.

### Statistical Analyses

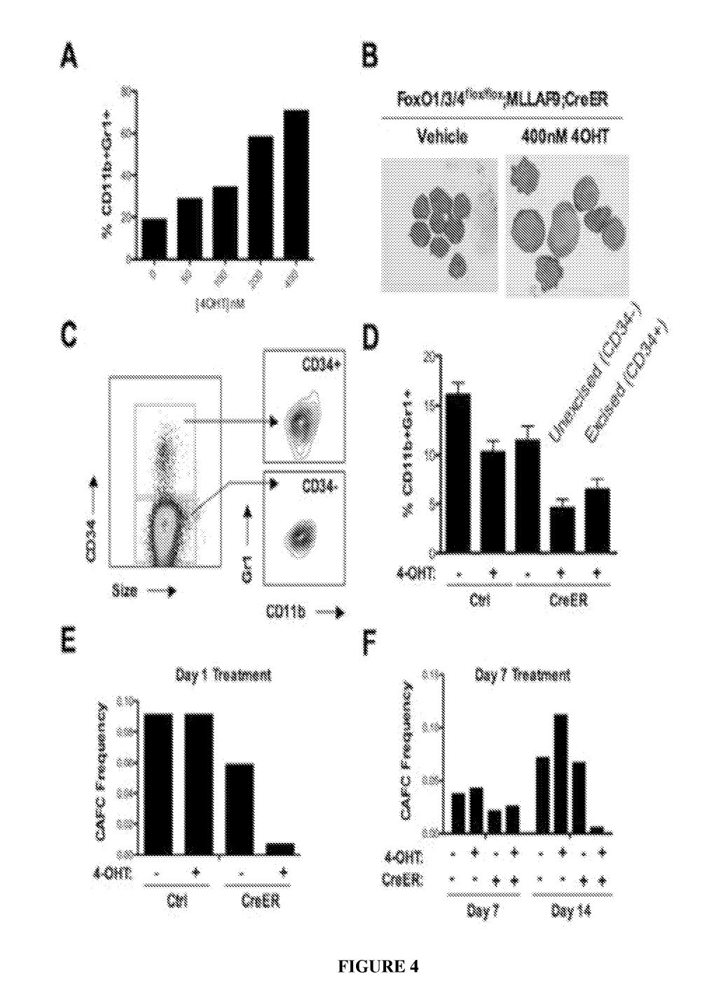

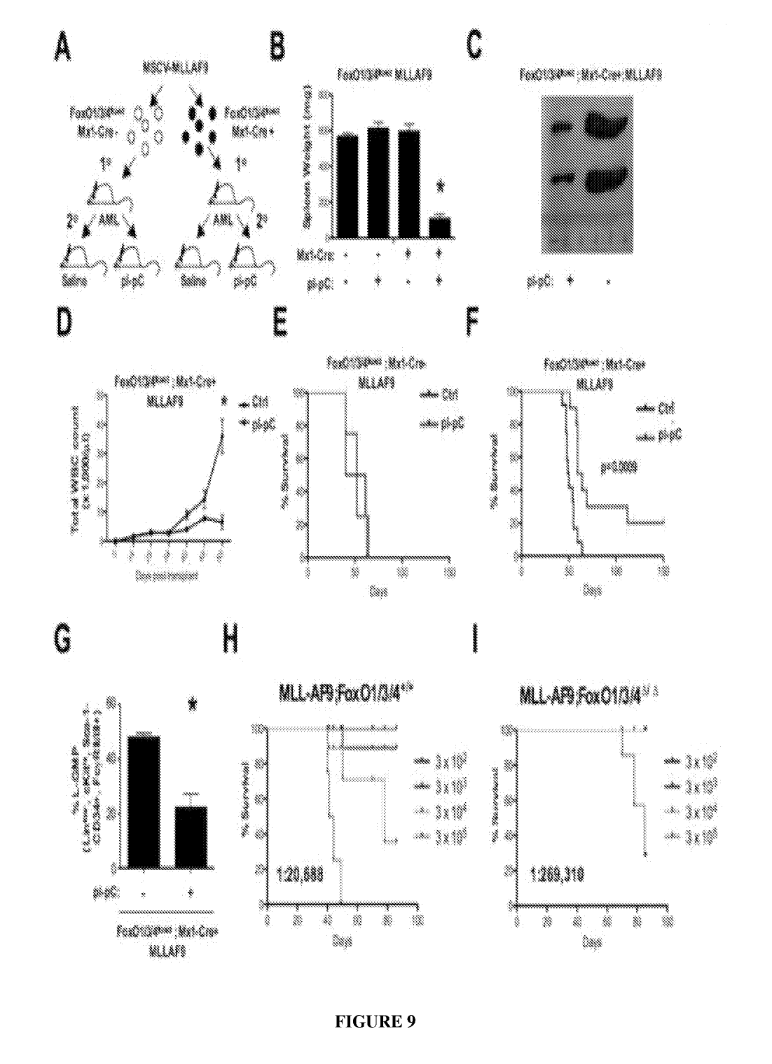

Log-Rank (Mantel-Cox) test was used to determine p values for all Kaplan-Meier survival curve analyses. Unpaired, two-tailed student's t tests were used for all analyses comparing two experimental groups. Poisson statistics was used to determine the leukemia-initiating cell frequency in vivo in FIGS. 9H and 9I as well as in vitro in FIGS. 4E and 4F.

### Example 2

### Exemplary Results

### AKT Activity is Diminished in MLL-AF9 CD34+ Myeloid Progenitors

Since specific modifications of AKT confer distinct clinical outcomes of human AML (Gallay et al., 2009; Park et al., 2009; Tamburini et al., 2007), AKT status was examined in a murine model of MLL-AF9-induced myeloid leukemia that closely phenocopies human AML (Krivtsov et al., 2006). In this model, the L-GMP (leukemia-granulocyte macrophage progenitor) cell population, which shares the same immunophenotype of GMPs (granulocyte macrophage progenitors; lineage^{low}, cKit^{hi}, Sca-1⁻, FcγRII/III⁺, CD34⁺) is enriched for leukemia-initiating cell (LIC) activity. AKT phosphorylation was assessed in lineage low cells purified from healthy and MLL-AF9-leukemic mice using multi-parameter fluorescence-activated cell sorting (FACS) and then rested ex vivo for 1 hour to allow signaling events between conditions to equilibrate. Cultivated cells were either left untreated or stimulated with cytokines to induce AKT phosphorylation. Cells from each condition were fixed, permeabilized and stained with cKit, Sca-1 and CD34 antibodies as well as antibodies that specifically recognize pAKT^{Ser473} and pAKT^{Thr308} epitopes.

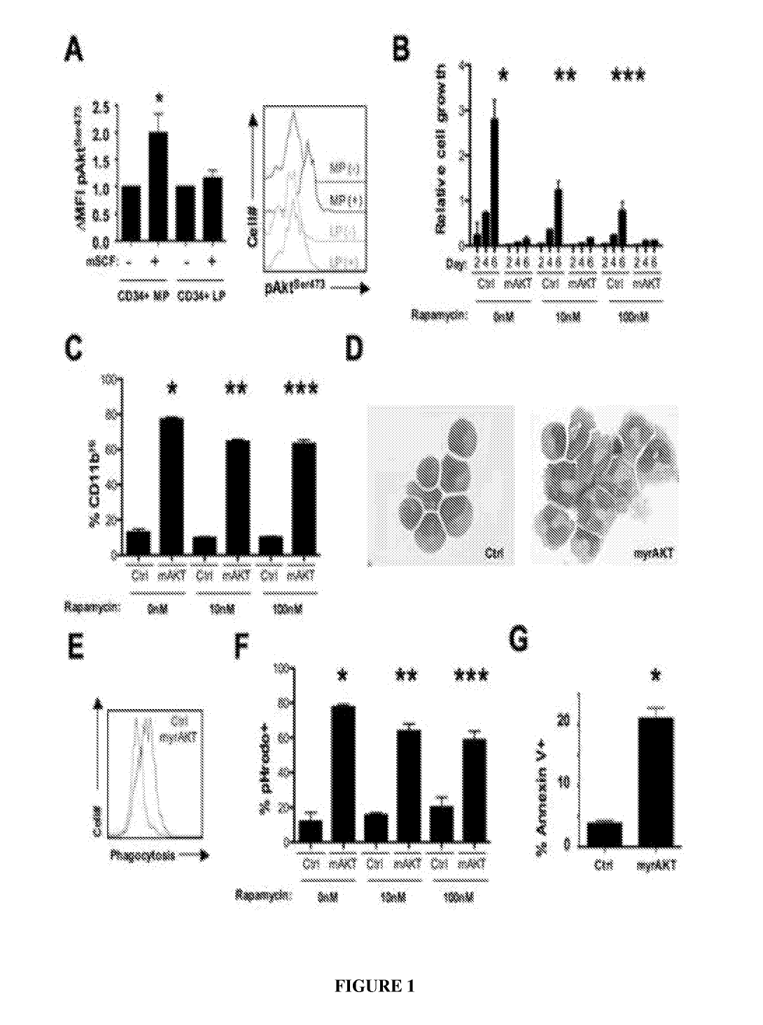

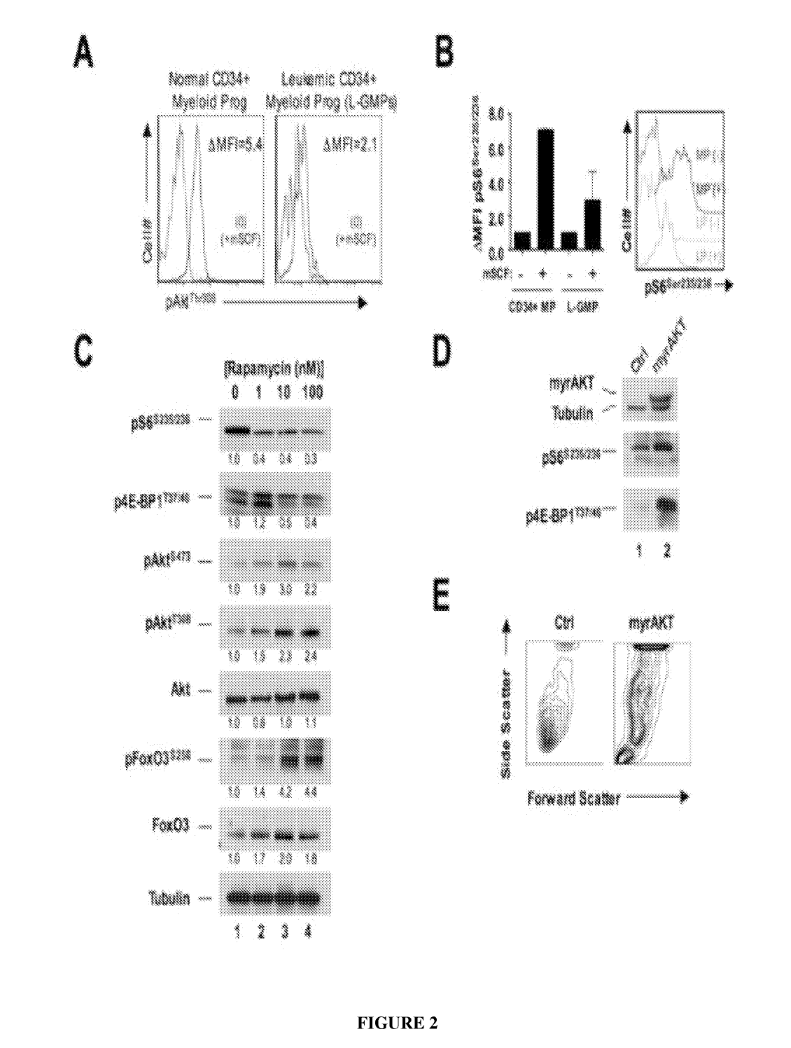

Normal CD34+ myeloid progenitors displayed a robust increase in both pAkt^{Ser473} and pAKT^{Thr308} (FIGS. 1A and 2A), however, CD34+ leukemic progenitors (enriched for L-GMPs) exhibited markedly reduced pAKT^{Ser473} and pAKT^{Thr308} in response to stimulation, indicating attenuated AKT activation (FIG. 1A and FIG. 2A). To further assess AKT signaling, CD34+ cells were also evaluated for serine 235/236 phosphorylation of ribosomal protein S6 (pS6^{Ser235/236}) a downstream effector of AKT signaling (Burgering and Coffer, 1995). Normal CD34+ cells showed strong induction of pS6^{Ser235/236} (FIG. 2B) while CD34+ leukemic progenitors had a blunted pS6^{Ser235/236} response, further demonstrating that AKT activity is diminished in MLL-AF9 LIC-enriched populations (FIG. 2B).

### Constitutive AKT Activation Promotes Myeloid Differentiation and Apoptosis of Murine AML Cells

The biological consequences of enforcing AKT activity in MLL-AF9-positive AML cells were further determined. To that end, BM cells from MLL-AF9 leukemic mice were adapted to liquid culture and infected with recombinant retroviruses expressing a constitutively active form of AKT (myrAKT). Control and myrAKT-infected cells were grown in cytokine-enriched media for six days and the myrAKT cells had markedly impaired proliferation (FIG. 1B), consistent with previous reports (Wang et al., 2008). Ablation of PTEN (an inhibitor of AKT signaling) in the murine hematopoietic system results in the rapid onset of myeloid and/or lymphoid neoplasia (Lee et al., 2010; Yilmaz et al., 2006). Within this model, leukemogenesis is largely dependent on mTOR signaling as rapamycin markedly delays disease onset (Yilmaz et al., 2006). Enforced expression of myrAKT strongly increased the phosphorylation of mTOR substates (FIG. 2D). However, treatment of myrAKT-expressing AML cells with concentrations of rapamycin that effectively inhibit mTOR signaling did not completely rescue the cell growth defect (FIGS. 1B and 2C). While these results do not exclude a role for mTOR signaling, they do indicate that additional mechanisms might contribute to the cell growth defect associated with myrAKT-expression in AML cells.

Expression of myrAKT in normal bone marrow reduces the self-renewal properties of normal hematopoietic stem and progenitor cells (HSPCs) and promotes myeloid maturation (Kharas et al., 2010b). Therefore, whether myrAKT expression was inhibiting AML cell growth by promoting myeloid maturation was examined. Myeloid blasts are smaller and less granular than mature myeloid cells. Flow cytometric analysis revealed that myrAKT cells displayed increased forward and side scatter indicating the cells were larger and more granular than control cells (FIG. 2E). Analysis of mature myeloid cell markers revealed higher levels of CD11b on myrAKT cells consistent with induction of myeloid lineage maturation (FIG. 1C). Correspondingly, myrAKT cells exhibited morphological changes of myeloid maturation such as reduced nucleoli, increased cytoplasmic volume, granule formation and condensed chromatin pattern (FIG. 1D). Finally, myrAKT cells acquired the ability to engulf fluorescent-labeled bacterial peptides confirming the myrAKT-directed maturation of leukemic blasts into functional myeloid cells with the capacity for phagocytosis (FIGS. 1E and 1F).

Upon completion of myelopoiesis, mature myelocytes have a limited lifespan in the peripheral blood. Therefore, whether myrAKT-induced maturation of myeloid cells is accompanied by increased cell death was examined. Myeloid cells expressing myrAKT displayed elevated levels of apoptosis consistent with constitutive activation of AKT promoting terminal maturation of leukemic cells (FIG. 1G). Maturation related death mediated by myrAKT occurred in the presence of rapamycin indicating that AKT utilizes pathways other than mTOR activation for myeloid maturation (FIGS. 1C-F). Together, these results indicate that LICs within this model maintain low levels of AKT activity to preserve an immature cell state and prevent differentiation related death.

### FoxOs are Active in Murine MLL-AF9 L-GMPs

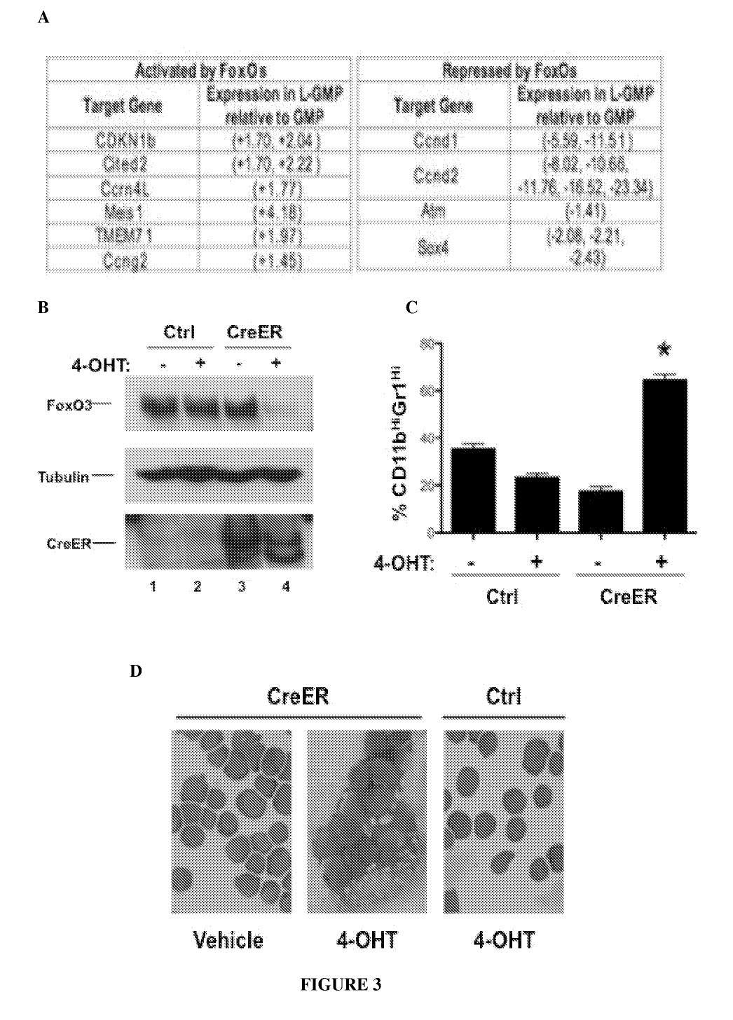

FoxOs play central roles in regulating normal hematopoiesis and the maintenance of hematopoetic stem and progenitor cell (HSPC) reserves and are integral mediators of AKT's actions in cellular growth and survival (Fu and Tindall, 2008; Miyamoto et al., 2007; Tothova et al., 2007; Yalcin et al., 2008). FoxO activity in L-GMPs was evaluated by assessment of several well-established FoxO target genes. Cdkn1b (p27), Cited2, Ccrn4l, Meis1, Tmem71 and Ccng2 are all activated by FoxOs and were found to be up-regulated in L-GMPs compared to GMPs (FIG. 3A). Ccnd1, Ccnd2, Atm and Sox4, which are repressed by FoxOs, were documented to be down-regulated in L-GMPs (FIG. 3A).

Since AKT-mediated phosphorylation of FOXOs leads to nuclear exclusion and subsequent inactivation, the nuclear localization of FoxO3 (the dominant FoxO family member active in the murine hematopoietic system (Miyamoto et al., 2007; Yalcin et al., 2008)), was evaluated in normal GMPs and L-GMPs (both lineage^{low}, cKit^{hi}, Sca-1⁻, FcγRII/III⁺, CD34⁺) using immunofluorescence. Although FoxO3 was predominantly cytoplasmic in GMPs, L-GMPs displayed nuclear localization of FoxO3 consistent with activation of FoxO (data not shown). Collectively, these data indicate that FoxOs are active in L-GMPs but does not exclude that it is also active in other AML cellular subsets.

### Deletion of FoxOs Promotes Myeloid Maturation of Murine AML Cells

Next, genetic evidence for an essential role of active FoxOs in the maintenance of the differentiation blockade of leukemic cells was examined. BM cells recovered from mice bearing homozygous floxed alleles of FoxO1, FoxO3 and FoxO4 (FoxO1/3/4^{floxed}) were infected with recombinant retroviruses expressing MLL-AF9. Transduced FoxO1/3/4^{floxed}; MLL-AF9 BM cells were subsequently transplanted into lethally irradiated primary recipients and monitored for the development of leukemia. Upon displaying signs of AML, mice were euthanized and assessed for leukemic burden. FoxO1/3/4^{floxed}; MLL-AF9 leukemic BM cells were then engineered to stably express a CreER fusion protein thus allowing for inducible excision of FoxO1/3/4^{floxed} alleles ex vivo (FIG. 3B).

To examine whether deletion of FoxO1/3/4 mimics the ability of myrAKT to promote myeloid maturation of leukemic cells, FoxO1/3/4^{floxed}; MLL-AF9 leukemic cells stably expressing CreER were cultured in the presence and absence of 4-OHT and monitored for changes in immunophenotype and morphology. Deletion of FoxO1/3/4 resulted in increased surface expression of the mature myeloid cell markers CD11b and Gr-1 (FIGS. 3C and 4A). Further, ablating FoxO1/3/4 induced morphological changes consistent with myeloid maturation (FIGS. 3D and 4B). The myeloid maturation observed with FoxO1/3/4 deletion was associated with gene ablation and not Cre expression alone (FIGS. 4C and 4D). Finally, deletion of FoxO1/3/4 diminished the ability FoxO1/3/4^{floxed}; MLL-AF9 leukemic cells to form and maintain colonies on supportive stroma (FIGS. 4E and 4F). Together, these data indicate that FoxO1/3/4 deficiency promotes myeloid maturation mirroring the activated AKT phenotype.

### Depletion of FOXO3 Promotes Myeloid Maturation and Apoptosis of Human AML Cell Lines

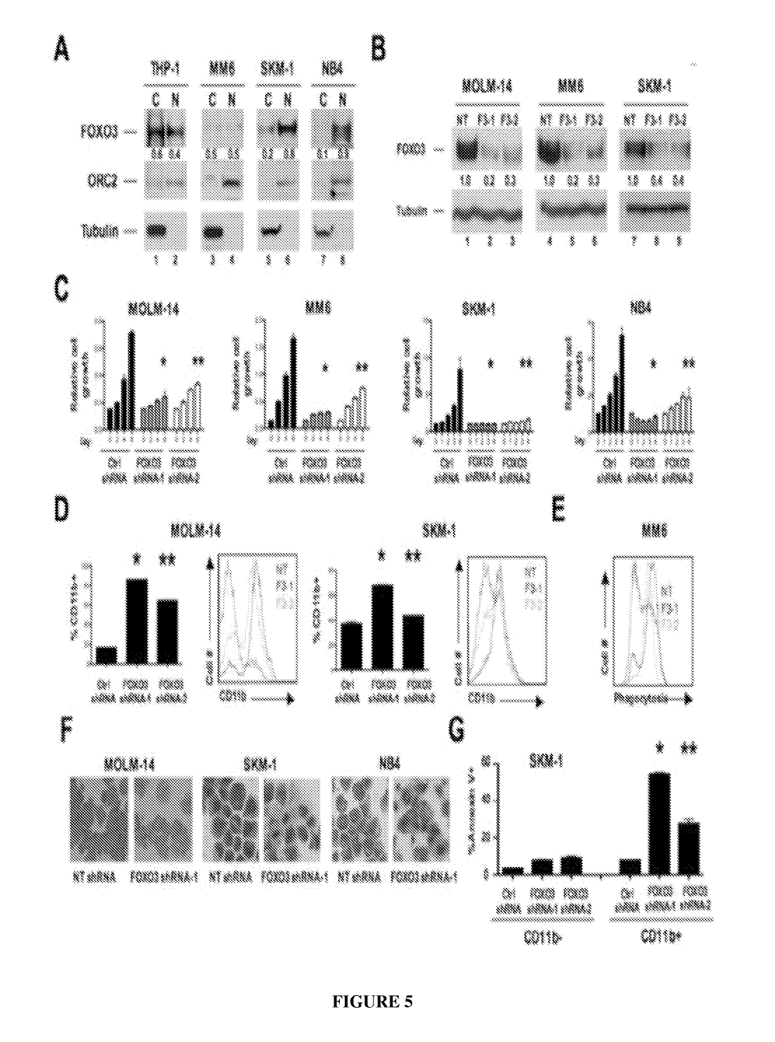

To determine whether FOXO3 is also active in human AML cell lines carrying MLL-AF9 translocations, the cytoplasmic and nuclear fractions of THP-1 and Mono-mac-6 (MM6) cells were evaluated for FOXO3 expression. Similar to the murine model, FOXO3 was nuclear in these human AML cell lines with approximately equal distribution between cellular compartments. Furthermore, FOXO3 localization was examined in a human AML cell line without an MLL-AF9 fusion gene (SKM-1 cell line) as well as a human acute promyelocytic leukemia (APL) cell line (NB4). SKM-1 and NB4 cells displayed 75% and 86% nuclear FOXO3, respectively, indicating that FOXO3 may serve comparable biological roles in other forms of AML (FIG. 5A). Together, these data show that FOXOs are active in human AML cells.

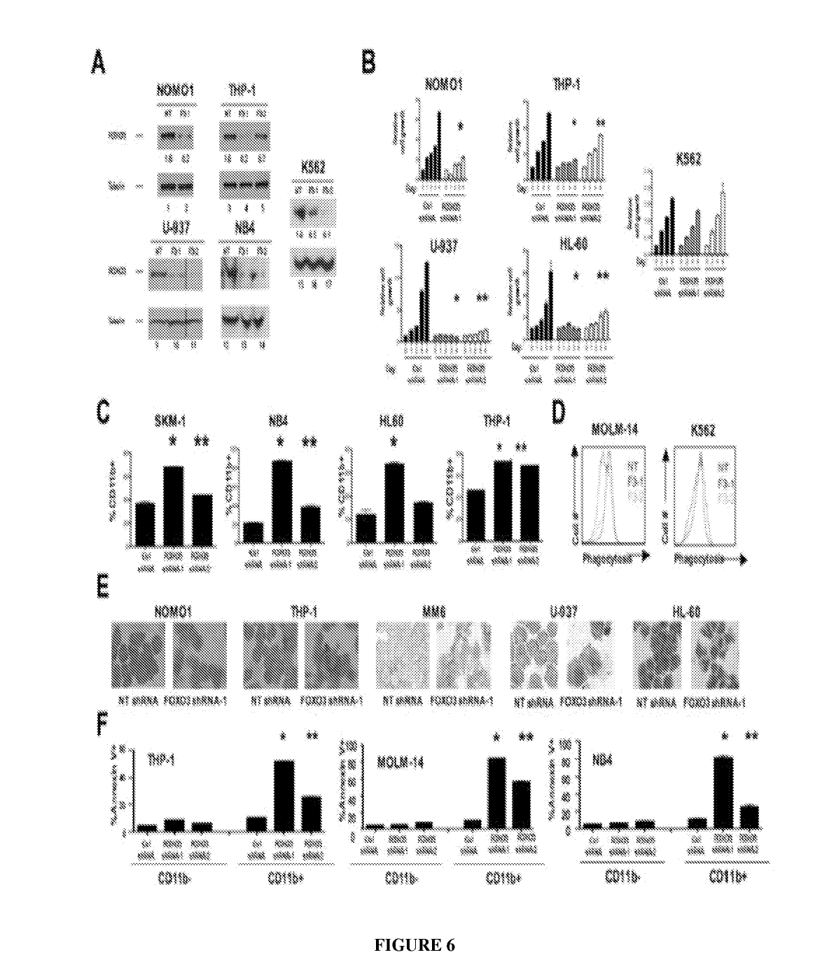

To determine whether FOXOs are required to support human AML growth, an shRNA-mediated approach was used to evaluate the role of FOXO3 in four cell lines with MLL-AF9 chromosomal translocations (MOLM-14, THP-1, Mono-mac-6 (MM6) and NOMO-1), two APL cell lines (HL-60 and NB4) and two AML cell lines that do not carry MLL-AF9 translocations (SKM-1 and U-937) (FIGS. 5B and 6A). All eight cell lines expressing FOXO3 shRNA exhibited lower cell growth rates in comparison with control shRNA-expressing cells (FIGS. 5C and 6B). Consistent with the effects seen with FoxO1/3/4 deletion in murine AML, depletion of FOXO3 resulted in increased mature myeloid surface marker (CD11b) expression (FIGS. 5D and 6C) and induced morphological changes consistent with myeloid cell maturation (FIGS. 5F, and 6E). Knockdown of FOXO3 also enhanced the phagocytic capabilities of MOLM-14 and Mono-mac-6 (MM6) cells (FIGS. 5E and 6D). MOLM-14 cells expressing FOXO3 shRNAs acquired increased levels of the monocytic marker, α-naphthylacetate esterase (α-NAE), though myeloperoxidase (MPO) activity apparent at baseline did not appear to change indicating FOXO3 inhibition promotes monocytic differentiation (data not shown). Finally, mature myeloid cells (CD11b+) expressing FOXO3 shRNAs displayed a marked increase in apoptosis confirming that depletion of FOXO3 initiates myeloid maturation that ultimately results in cell death (FIGS. 5G and 6F).

Contrary to the other human AML cell lines examined, depletion of FOXO3 in the BCR-ABL-positive cell line did not reduce cell numbers, increase mature myeloid surface markers or alter the phagocytic properties of K562 cells, indicating that either FOXO signaling is not required or another FOXO family member is needed to maintain these cells (FIGS. 6A, 6B and 6D).

FOXO3 is Activated in Primary AMLs Derived from Patients

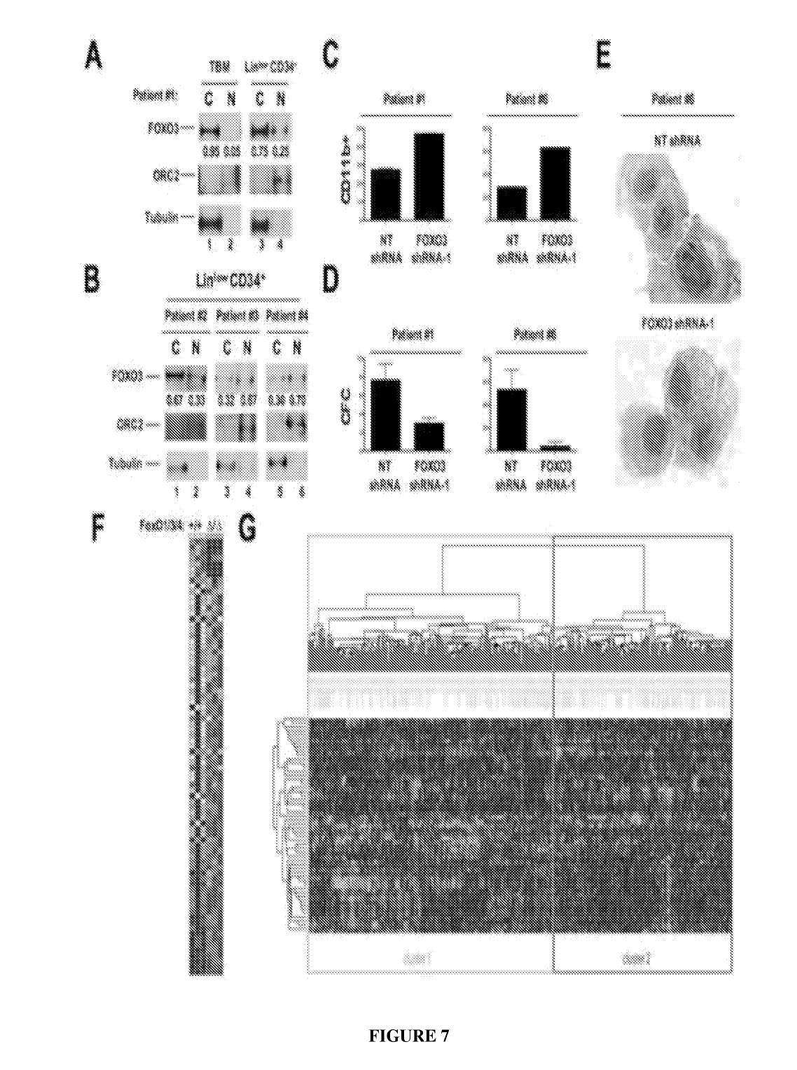

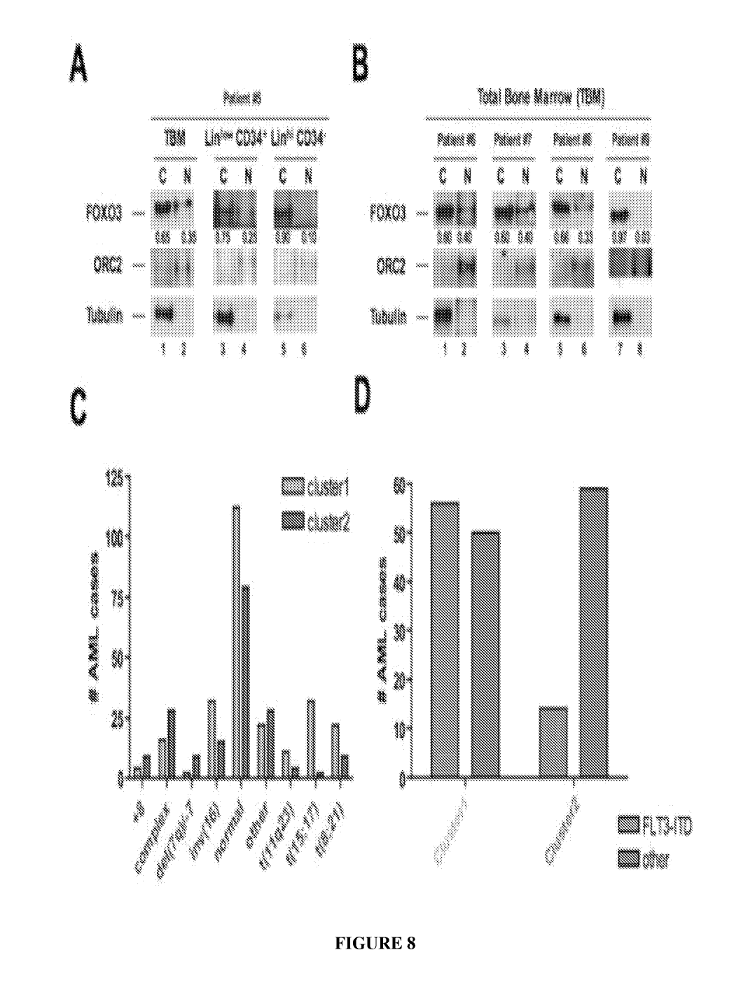

To validate the findings in AML cell lines, the cellular distribution of FOXO3 was examined in primary bone marrow (BM) cells derived from nine different AML patients. Biochemical fractionation displayed a wide variation between samples in the ratio of nuclear-to-cytoplasmic ratio of FOXO3 (3-70% nuclear FOXO3) (FIGS. 7A, 7B, 8A and 8B). Five of the nine samples contained CD34 positive blasts. FACS isolation of CD34 positive, lineage low population revealed enrichment for nuclear FOXO3 in comparison with fractionation of the total BM or CD34-, lineage high cells (FIGS. 7A and 8A). Within these five samples the overall level of nuclear FOXO3 varied between 10-70%. Two samples formed colonies in methylcellulose allowing the introduction of shRNAs. Depletion of FOXO3 reduced colony formation and promoted myeloid maturation (based on surface marker expression and morphology) in both samples (FIGS. 7C-7E).

To determine the global status of FOXO activity in AML, gene expression array data from murine HSCs with and without FoxO1/3/4 was utilized to generate a hematopoietic-specific gene signature of FOXO activity (FIG. 7F) (Tothova et al., 2007). Using this FoxO target gene set, unsupervised hierarchical clustering analysis of 436 primary AML samples was performed (Bullinger et al., 2004; Kharas et al., 2010a).

TABLE 3
Comprehensive Gene Set

| Probe Set | Gene Symbol | Probe Set | Gene Symbol |
|---|---|---|---|
| 1449526_a_at | Gdpd3 | 1451156_s_at | Vldlr |
| 1453109_at | Arsk | 1419202_at | Cst7 |
| 1456753_at | | 1439589_at | |
| 1443504_at | E330022O07 | 1458524_at | Fndc3a |
| 1455292_x_at | | 1441693_at | Adamts3 |
| 1428719_at | 2010309G21Rik | 1459794_at | |
| 1428720_s_at | 2010309G21Rik | 1448995_at | Pf4 |
| 1436491_at | OTTMUSG00000000601 | 1435665_at | Al451617 |
| 1423890_x_at | Atp1b1 | 1457477_at | |
| 1433028_at | 4833411l10Rik | 1446484_at | |
| 1442549_at | Mbnl3 | 1446508_at | |
| 1435953_at | Btaf1 | 1427351_s_at | Igh-6 |
| 1424810_at | Tasp1 | 1416700_at | Rnd3 |
| 1438781_at | Tet2 | 1431213_a_at | LOC67527 |
| 1422706_at | Pmepa1 | 1443703_at | Cd28 |
| 1427229_at | Hmgcr | 1438831_at | Crkrs |
| 1447517_at | Skiv2l2 | 1439946_at | |
| 1418997_at | Lyrm5 | 1429672_at | 5830407E08Rik |
| 1457088_at | Pldn | 1442704_at | |
| 1457108_at | | 1453841_at | 2310050P20Rik |
| 1439826_at | Hspa14 | 1445068_at | Malt1 |
| 1418203_at | Pmaip1 | 1450330_at | Il10 |
| 1448749_at | Plek | 1419766_at | Sik1 |
| 1459793_s_at | Lyrm5 | 1429381_x_at | Igh |
| 1446272_at | Pctk2 | 1416111_at | Cd83 |
| 1457445_at | Trps1 | 1435448_at | Bcl2l11 |
| 1455166_at | Arl5b | 1425472_a_at | Lmna |
| 1456686_at | | 1437025_at | Cd28 |
| 1435679_at | Optn | 1441172_at | |
| 1440651_at | | 1427242_at | Ddx4 |
| 1425407_s_at | Clec4a2 | 1457670_s_at | Lmna |
| 1423722_at | Tmem49 | 1453752_at | Rpl17 |
| 1419706_a_at | Akap12 | 1449991_at | Cd244 |
| 1444524_at | LOC100043424 | 1422412_x_at | Ear3 |
| 1454905_at | Ibtk | 1460423_x_at | Igkv1-117 |
| 1421031_a_at | 2310016C08Rik | 1422411_s_at | Ear3 |
| 1437074_at | Snx5 | 1446130_at | |
| 1452217_at | Ahnak | 1449965_at | Mcpt8 |
| 1419700_a_at | Prom1 | 1427858_at | Igkv1-117 |
| 1425223_at | Birc3 | | |

The differential expression of FOXO-target genes stratified AML into two distinct clusters strongly indicating that these clusters reflect two distinct patterns of FOXO activity; Cluster 1 AMLs display lower FOXO activity relative to AML within cluster 2 (FIG. 7G). Consistent with this notion, FOXO1 and FOXO3 expression in cluster 2 (higher FOXO activity) is significantly elevated compared to cluster 1 (lower FOXO activity) (p<0.0001, FIG. 7C). These data demonstrate that AML can be segregated into two distinct patterns of FOXO activity (FIG. 7G). Each cluster was significantly represented in nine AML sub-groups separated on the basis of defined chromosomal aberrations indicating that FOXO activation is not restricted to a particular sub-type of AML, although there is significant association with some of them (p<0.0001; FIG. 8C). For example, AMLs bearing FLT3-ITD mutations (associated with poor prognosis) were under represented in the cluster 2 (higher FOXO activity) gene signature (p<0.0001; FIG. 8D). Combined, these analyses extend the importance of FOXO3 beyond MLL-AF9-positive AMLs and indicate that FOXO3 may impact a broad spectrum of myeloid leukemias of various genotypes.

### MLL-AF9 Leukemic Burden is Reduced in the Absence of FoxO1/3/4 In Vivo

To address whether FoxO1/3/4 are essential to maintain MLL-AF9 induced AML in vivo, a MLL-AF9 bone marrow transplant (BMT) assay was used in a mouse bearing homozygous Lox-P flanked (floxed) alleles of FoxO1/3/4 and the interferon-inducible Mx1-Cre transgene (FoxO1/3/4^{floxed}; Mx1-Cre+; MLL-AF9). MLL-AF9 transformed FoxO1/3/4^{floxed} BM cells without the Mx1-Cre transgene (FoxO1/3/4^{floxed}; Mx1-Cre−; MLL-AF9) were used as controls. Leukemia cells from each context (recovered from primary recipients displaying frank leukemia) were harvested and transplanted into sub-lethally irradiated recipients. Fourteen days post-transplant, secondary recipients receiving FoxO1/3/4^{floxed}; Mx1-Cre+; MLL-AF9 cells or FoxO1/3/4^{floxed}; Mx1-Cre−; MLL-AF9 controls were either administered polyinosine-polycytidyline (pI-pC) to induce excision of FoxO1/3/4 floxed alleles or saline control (FIG. 9A). At the first presentation of AML, all mice from each group (Mx1-Cre− versus Mx1-Cre+) were euthanized and assessed for leukemic burden as defined by spleen weight and peripheral white blood cell (WBC) counts. Saline and pI-pC treated FoxO1/3/4^{floxed}; Mx1-Cre−; MLL-AF9 recipients as well as saline treated FoxO1/3/4^{floxed}; Mx1-Cre+; MLL-AF9 recipients all developed splenomegaly and leukocytosis (FIGS. 9B-D and 10A). In contrast, pI-pC-treated FoxO1/3/4^{floxed}; Mx1-Cre+; MLL-AF9 transplant mice retained normal spleen weights and WBC counts (FIGS. 9B-D and 10A). Furthermore, peripheral blood analysis of mature myeloid and lymphoid lineages revealed that pI-pC-treated FoxO1/3/4^{floxed}; Mx1-Cre+; MLL-AF9 mice exhibited normal lymphoid-myeloid ratio where all other conditions showed an over-abundance of myeloid cells (data not shown).

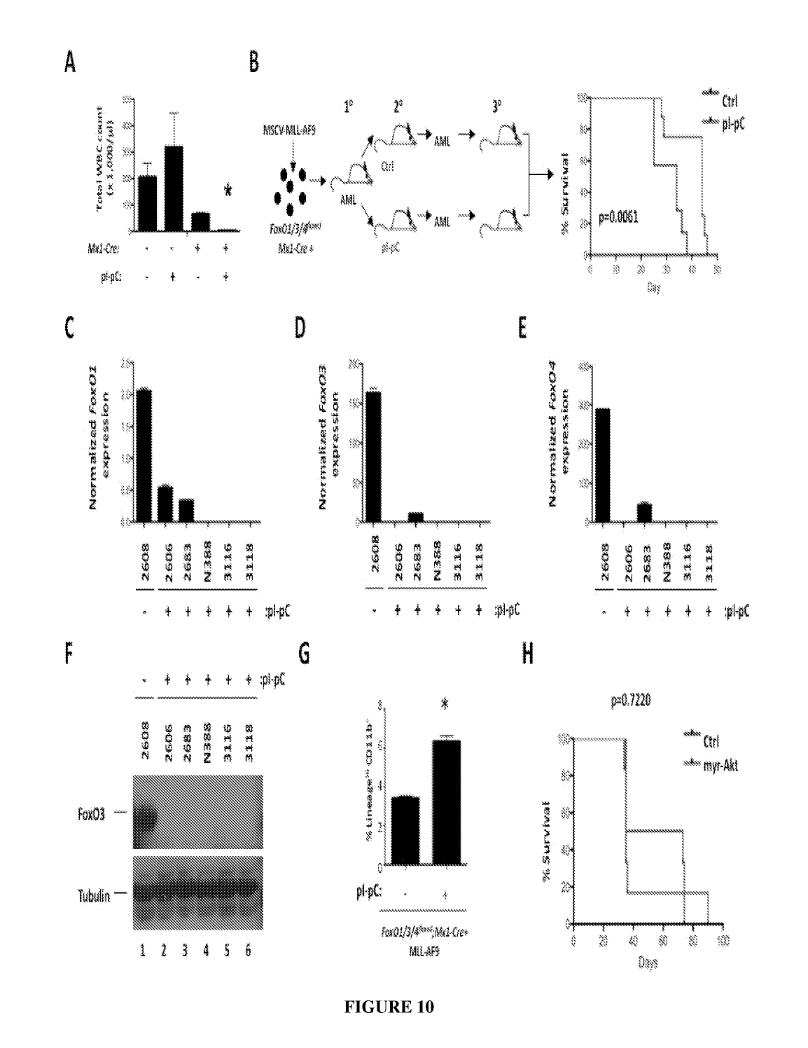

To distinguish whether disrupting FoxO1/3/4 expression eliminated MLL-AF9 leukemia or merely delayed the onset, FoxO1/3/4^{floxed}; Mx1-Cre−; MLL-AF9 and FoxO1/3/4^{floxed}; Mx1-Cre+; MLL-AF9 leukemic cells were separately transplanted into sub-lethally irradiated recipients. Fourteen days post-transplant, mice were administered either pI-pC or saline and subsequently monitored daily for clinical signs of leukemia. Peripheral blood from recipient mice was collected and analyzed for WBC counts every 4-14 days. Mice were euthanized upon displaying external morbidity and/or elevated WBC counts. Consistent with the prior findings, pI-pC-treated FoxO1/3/4^{floxed}; Mx1-Cre+; MLL-AF9 mice had lower WBC counts than saline-treated controls (FIG. 9D). Regardless of pI-pC treatment, recipient mice transplanted with FoxO1/3/4^{floxed}; Mx1-Cre−; MLL-AF9 leukemic cells (FIG. 9E) and recipient mice transplanted with FoxO1/3/4^{floxed}; Mx1-Cre+; MLL-AF9 cells that were treated with saline (FIG. 9F) had a similar latency for the onset of AML. In contrast, mice transplanted with FoxO1/3/4^{floxed}; Mx1-Cre+; MLL-AF9 leukemic cells and treated with pI-pC, resulting in excision of FoxO1/3/4, displayed a significantly longer latency. Interestingly, 20% of mice (n=10) were disease-free up to 5 months post-excision. However, despite efficient excision of FoxO1/3/4 (FIGS. 10C-10F) the majority of mice eventually succumbed to leukemia (FIG. 9F). Collectively, these in vivo studies show that one or more of FoxO1/3/4 support MLL-AF9-induced leukemia.

### FoxO1/3/4 are Required for LIC Function In Vivo

The leukemia-initiating cell (LIC) population represents a small subset of AML cells that retain the ability to give rise to leukemia in recipient mice. As noted, the L-GMP population (lineage^{low}, cKit^{hi}, Sca-1⁻, FcγRII/III⁺, CD34⁺) of this AML murine model is enriched for LIC activity. Therefore, we set out to directly address the role of FoxO1/3/4 in LIC activity. To this end, mice transplanted with FoxO1/3/4^{floxed}; Mx1-Cre+; MLL-AF9 leukemic cells were administered pI-pC or saline 14 days post transplantation. Seven days following pI-pC treatment, mice were euthanized and BM cells from each condition were analyzed for LIC activity. FoxO1/3/4 ablation resulted in an immunophenotypic reduction of L-GMPs and a concomitant increase of lineage^{hi}, CD11b^{hi(+)} myeloid cells (FIGS. 9G and 10G).

To test whether loss of FoxO1/3/4 also diminished LIC function, a limited dilution assay was performed for leukemia cells immediately following FoxO1/3/4 deletion. Briefly, equal numbers of leukemic BM cells were recovered from FoxO1/3/4^{floxed}; Mx1-Cre+; MLL-AF9 mice seven days following pI-pC or saline treatment. Recipient mice were then injected with 300, 3,000, 30,000 or 300,000 cells of each condition. Ninety days following transplantation, 100%, 75% and 17% of mice that received 300,000, 30,000 or 3,000 cells, respectively, from saline-treated FoxO1/3/4^{floxed}; Mx1-Cre+; MLL-AF9 succumbed to AML corresponding to a LIC frequency of 1:20,688 (FIG. 9H). In contrast, 75% of mice transplanted with 300,000 pI-pC-treated FoxO1/3/4^{floxed}; Mx1-Cre+; MLL-AF9 donor cells developed AML whereas 100% of mice from each other cell dose were alive at day 90. Based on these results, it was found that FoxO1/3/4 deletion reduced the LIC frequency to 1:269,310, which is 13-fold lower than the control group (FIG. 9I). Furthermore, mice transplanted with 300,000 pI-pC-treated FoxO1/3/4^{floxed}; Mx1-Cre+; MLL-AF9 donor cells (median survival 82 days, FIG. 9I) displayed nearly a 2-fold increase in median survival over mice transplanted with saline-treated FoxO1/3/4^{floxed}; Mx1-Cre+; MLL-AF9 donor cells (median survival 43 days, FIG. 9H) consistent with FoxO1/3/4 significantly extending the latency of MLL-AF9-positive AML in vivo (n=4, p=0.0007). Additionally, mice reconstituted with donor cells from pI-pC-treated FoxO1/3/4^{floxed}; Mx1-Cre+; MLL-AF9 mice from FIG. 9F displayed an extended latency in comparison with recipients transplanted with donor cells from saline-treated FoxO1/3/4^{floxed}; Mx1-Cre+; MLL-AF9 leukemic mice in FIG. 9F (FIG. 10B). Taken together, these data demonstrate that in vivo maximal LIC activity is dependent on functional FoxO1/3/4 expression.

Constitutive Activation of AKT does not Alter Disease Latency In Vivo

Based on the observations that AKT reduced MLL-AF9 cell growth in vitro and deletion of FoxO1/3/4 extends disease onset in vivo, we examined the effects of expressing myrAKT in MLL-AF9-induced leukemia cells in vivo. BM cells derived from mice transformed with MLL-AF9 were infected with either control or myrAKT recombinant retroviruses that also express GFP. Following infection, GFP positive cells from each condition were isolated by FACS and separately injected into syngeneic recipients. Mice from all conditions succumbed to AML and no significant difference in latency between control and myrAKT-expressing AMLs were observed (FIG. 10H). The lack of impact on latency was unexpected and caused us to examine whether a mechanism of resistance to AKT activation had emerged.

### Inhibition of FOXO3 Activates JNK/c-JUN Signaling

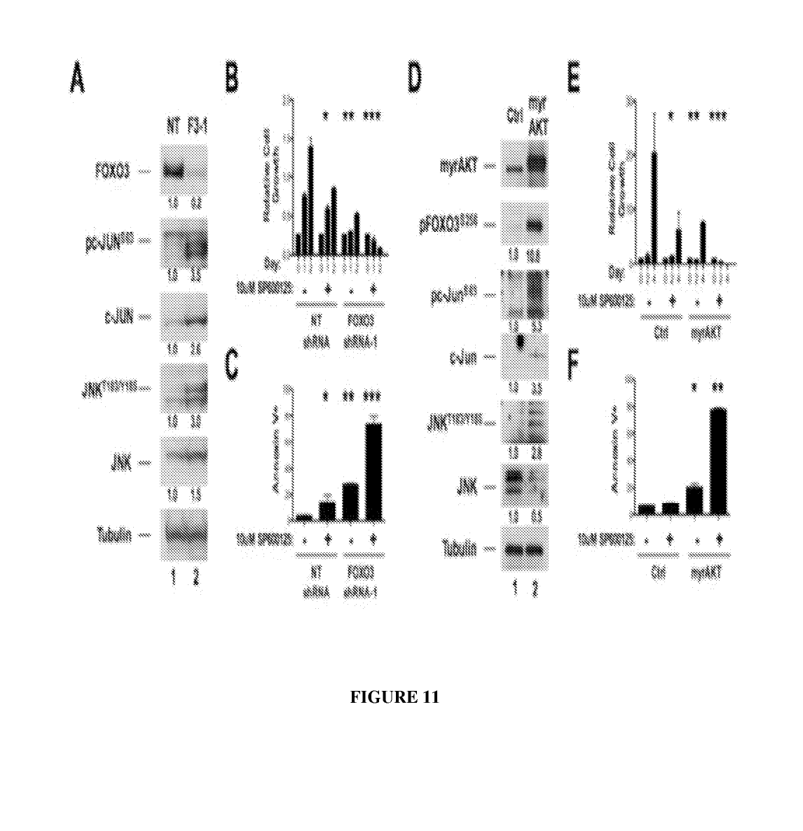

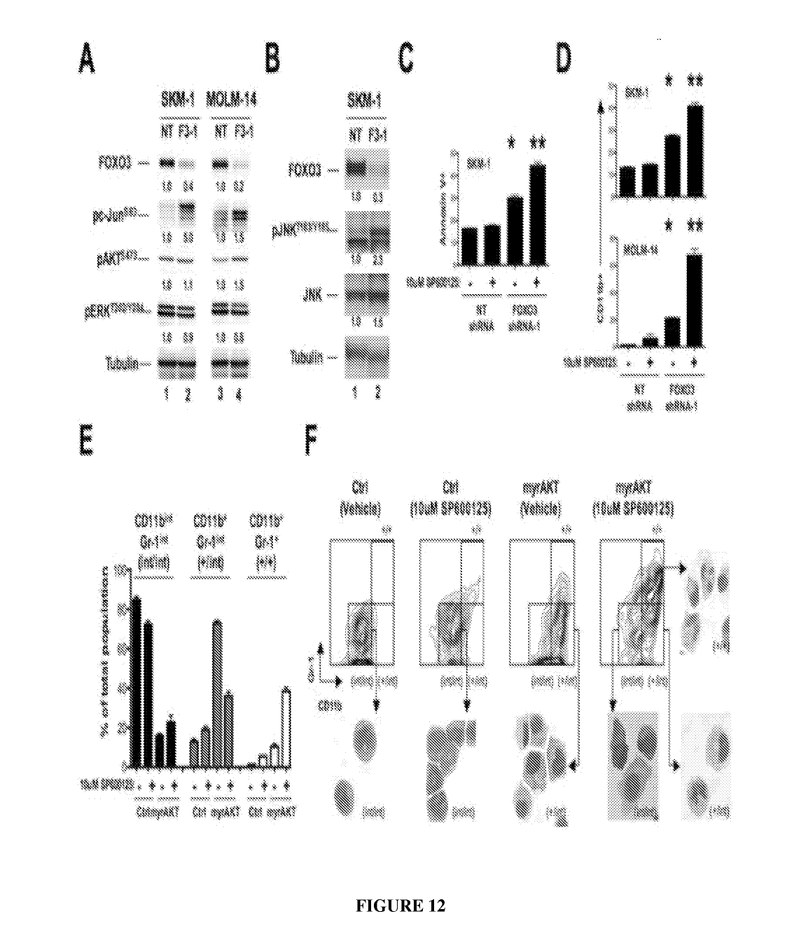

To evaluate the downstream molecular events that regulate myeloid maturation of leukemic cells in response to FOXO inhibition, the status of multiple signaling pathways involved in myeloid differentiation were examined. Phosphorylation of ERK, AKT and c-JUN were assessed upon shRNA-mediated inhibition of FOXO3 expression in MOLM-14 and SKM-1 cells. While FOXO3 depletion did not have a substantial effect on AKT or ERK phosphorylation, Serine 63 phosphorylation within c-JUN (pc-JUN^{Ser63}) was elevated in both cell lines (FIG. 12A). The c-JUN oncogene is a member of the AP-1 family of transcription factors that is phosphorylated and activated by the stress-activated kinase, SAPK/JNK (hereafter JNK) (Derijard et al., 1994; Hibi et al., 1993). Under various forms of stress, JNK is phosphorylated at threonine 183/tyrosine 185 (pJNK^{Thr183/Tyr185}) (Derijard et al., 1994). Depletion of FOXO3 resulted in substantial increases in pJNK^{Thr183/Tyr185}, pc-JUN^{Ser63} and total c-JUN levels (FIGS. 11A and 12B). Enforced expression of myrAKT in murine MLL-AF9-expressing AML cells also resulted in increases in pJnk^{Thr183/Tyr185}₉ pc-Jun^{Ser63} and total c-Jun levels (FIG. 11D). This was seen in the context of inhibition of FoxO3 as evident by the substantial increase in phosphorylation of FOXO3^{Ser256} (pFoxO3^{Ser256}) (FIG. 11D). These data indicate an activation of JNK/c-JUN with FOXO inhibition (either directly or via AKT activation) and raised the question of whether JNK/c-JUN participate in leukemia resistance to the latter interventions.

Pharmacological Inhibition of JNK Cooperates with FOXO Inhibition to Induce AML Cell Death

Whether JNK/c-JUN signaling has a functional impact on cellular events induced by AKT and FOXO signaling was tested. A pan JNK inhibitor (SP600125, CALBIOCHEM®) was combined with either FOXO3 depletion or myrAKT expression and the growth, differentiation and survival of AML cells was evaluated. Treatment of control infected MOLM-14, SKM-1 or MLL-AF9 expressing murine leukemia cells with SP600125 caused a modest but significant decrease in cell growth (FIGS. 11B and 11E). Inhibition of JNK also stimulated a mild increase in mature myeloid surface marker expression and apoptosis (FIGS. 11C, 11F, 12C-12E). As seen previously, depletion of FOXO3 or myrAKT resulted in significant decreases in cell growth and increases in mature myeloid surface marker expression and apoptosis (FIGS. 11C, 11F, 12-E). Notably, the combination of SP600125 with either FOXO3 depletion or myrAKT expression led to a significant decrease in cell growth compared to FOXO inhibition or SP600125 treatment alone (FIGS. 11B and 11E). The combination of SP600125 and FOXO inhibition (either FOXO3 shRNA or myrAKT expression) also increased apoptosis (FIGS. 11C and 11F) and mature myeloid surface marker expression (FIGS. 12D-12F) above that of FOXO3 inhibition or SP600125 treatment alone. Furthermore, SP600125 combined with myrAKT resulted in morphological changes associated with greater degree of myeloid differentiation compared to each alone (FIG. 12F). Taken together, these data indicate that FOXO3 inhibition or AKT activation in AML activates the JNK/c-JUN pathway. JNK activation may partially mitigate the anti-leukemic properties of FOXO3 inhibition or AKT activation. Combining JNK inhibition with FOXO3 inhibition or AKT activation more effectively enables leukemia inhibition.

c-JUN Activity is Elevated in AMLs with Diminished FOXO Activity

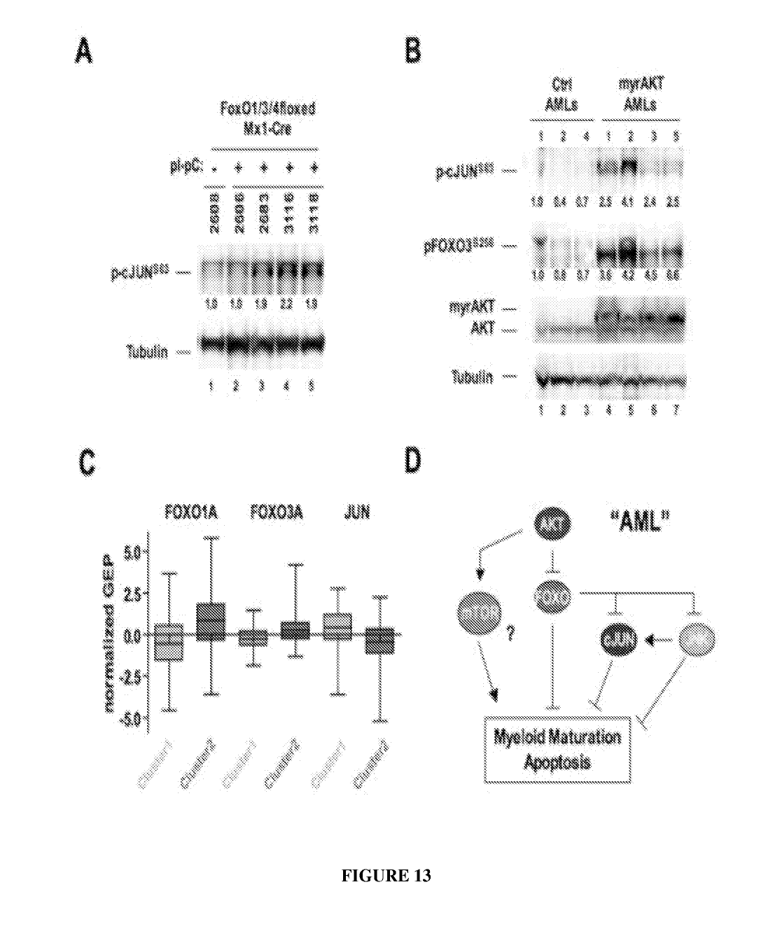

Based on the observation that the JNK/c-JUN signaling pathway is activated in response to FOXO inhibition (directly or via AKT activation), whether the activation of this pathway was also initiated in vivo was examined. To this end, the phosphorylation status of c-Jun in BM cells of saline and pI-pC-treated FoxO1/3/4^{floxed}; Mx1-Cre+; MLL-AF9 mice (FIG. 9F) that developed overt AML was examined. Enhanced pc-Jun^{Ser63} in 75% of mice that died of AML despite efficient deletion of FoxO1/3/4 (FIGS. 13A and 10C-10F) was observed. The phosphorylation status of BM cells derived from control and myrAKT-expressing leukemic mice from FIG. 10H was evaluated. Leukemic BM from 4 mice that succumbed to AML expressing MLL-AF9 and myrAKT displayed elevated pc-Jun^{Ser53} compared to control leukemic BM (FIG. 13B). Furthermore, all 4 myrAKT-expressing AMLs displayed increases in pFoxO3^{Ser256} confirming that myrAKT was inhibiting FoxO3 in these tumors (FIG. 13B).

FOXO and c-JUN Display Inverse Activities in Primary Human AML

To get a broader understanding of the relationship between FOXO inhibition and JNK/c-JUN activation in human AML, the expression of c-JUN was determined in the gene expression array data set generated from 436 primary AML patient samples. c-JUN expression inversely correlated with FOXO1 and FOXO3 expression (p<0.0001; FIG. 13C). Furthermore, c-JUN expression was elevated in cluster 1, which is associated with lower FOXO activity in comparison with cluster 2 (high FOXO activity) (FIG. 13C). These data indicate that activation of the JNK/c-JUN pathway represents a common mechanism by which AML compensates for the deregulation of FOXO/AKT signaling and offers a novel therapeutic opportunity of targeting dysregulated JNK/c-JUN signaling in FOXO/AKT-deregulated AML. Collectively, these data demonstrate that inhibition of the AKT/FOXO signaling pathway preserves the immature phenotype and the leukemia-initiating properties of various AML subtypes. Furthermore, AML cells retain a compensatory molecular switch (activation of JNK/c-JUN signaling) that allows them survive and propagate in the absence of FOXOs.

TABLE 4
Exemplary RNAi Sequences targeted against human FOXO
transcription factors FOXO1, FOXO3, and FOXO4.

| | | SEQ | | SEQ |
|---|---|---|---|---|
| Name | Sense Seq. | ID NO: | Antisense Seq. | ID NO: |
| human | GAGCGTGCCCTACTTCAAG | 11 | CTTGAAGTAGGGCACGCTC | 12 |
| FOXO | | | | |
| 1 RNA | | | | |
| seq #1 | | | | |
| human | TCTCCTAGGAGAAGAGCTG | 13 | CAGCTCTTCTCCTAGGAGA | 14 |
| FOXO | | | | |
| 1 | | | | |
| RNAi | | | | |
| seq #2 | | | | |
| human | CAGACCCTCAAACTGACACAA | 15 | TTGTGTCAGTTTGAGGGTCTG | 16 |
| FOXO | | | | |
| 3 | | | | |
| RNAi | | | | |
| seq #1 | | | | |
| human | CTCCTTTAACAGCACGGTGTT | 17 | AACACCGTGCTGTTAAAGGAG | 18 |
| FOXO | | | | |
| 3 | | | | |
| RNAi | | | | |
| seq #2 | | | | |
| human | TCTCACCTCTTCCCATTCC(dTdT) | 19 | GGAATGGGAAGAGGTGAGA(dTdT) | 20 |
| FOXO | | | | |
| 4 | | | | |
| RNAi | | | | |
| seq #1 | | | | |
| human | AGAAGCCGATATGTGGACC(dTdT) | 21 | GGTCCACATATCGGCTTCT(dTdT) | 22 |
| FOXO | | | | |
| 4 | | | | |
| RNAi | | | | |
| seq #2 | | | | |

Exemplary RNAi agents for FOXO6 can be purchased commercially from e.g., SANTA CRUZ BIOTECHNOLOGIES™, and SIGMA ALDRICH®. RNAi agents for FOXO1, FOXO3, FOXO4, and FOXO6 can also be obtained commercially from e.g., THERMO SCIENTIFIC DHARMACON®, and INVITROGEN™.

(myrAkt amino acid sequence)
SEQ ID NO: 26
-GSSKSKPKDPSQRR-MSDV-
GAEEMEVSLAKPKHRVTMNEFEYLKLLGKGTFGKVILVKEKATGRYYAM
KILKKEVIVAKDEVAHTLTENRVLQNSRHPFLTALKYSFQTHDRLCFVM
EYANGGELFFHLSRERVFSEDRARFYGAEIVSALDYLHSEKNVVYRDLK
LENLMLDKDGHIKITDFGLCKEGIKDGATMKTFCGTPEYLAPEVLEDND
YGRAVDWWGLGVVMYEMMCGRLPFYNQDHEKLFELILMEEIRFPRTLGP
EAKSLLSGLLKKDPKQRLGGGSEDAKEIMQHRFFAGIVWQHVYEKKSET
DTRYFDEEFTAQMITITPPDQDDSMECVDSERRPHFPQFSYSASSTA

Generation of a myrAkt expression plasmid is described in detail by Kohn, A D., et al., Journal of Biological Chemistry 271(36):21920-21926 (1996), which is herein incorporated by reference in its entirety.

The references cited herein and throughout the specification are incorporated herein by reference.

### REFERENCES

Alessi, D. R., et al. (1996). The EMBO journal 15, 6541-6551.
Alessi, D. R., et al. (1997). et al., Curr Biol 7, 261-269.
Altomare, D. A., and Testa, J. R. (2005). Oncogene 24, 7455-7464.
Arden, K. C. (2006). Experimental gerontology 41, 709-717.
Armstrong, S. A., et al. (2003). Seminars in hematology 40, 268-273.
Brunet, A., et al. (1999). Cell 96, 857-868.
Bullinger, L., et al. (2004). NEJM 350, 1605-1616.
Burgering, B. M. (2008). Oncogene 27, 2258-2262.
Burgering, B. M., and Coffer, P. J. (1995). Nature 376, 599-602.
Carter, M. E., and Brunet, A. (2007). Curr Biol 17, R113-114.
Cross, D. A., et al. (1995). Nature 378, 785-789.
Daser, A., and Rabbitts, T. H. (2005). Seminars in cancer biology 15, 175-188.
Dash, A., and Gilliland, D. G. (2001). Best practice & research 14, 49-64.
Datta, S. R., et al. (1997). Cell 91, 231-241.
del Peso, L., et al. (1997). Science 278, 687-689
Derijard, B., et al. (1994). Cell 76, 1025-1037.
Dohner, H. et al. (2010). Blood 115, 453-474.
Engelman, J. A. (2009). Nature reviews 9, 550-562.
Essers, M. A., et al. (2004). The EMBO journal 23, 4802-4812.
Franke, T. F. (2008). Oncogene 27, 6473-6488.
Frohling, S., et al. (2005). J Clin Oncol 23, 6285-6295.
Fu, Z., and Tindall, D. J. (2008). Oncogene 27, 2312-2319.
Gallay, N., et al. (2009). Leukemia 23, 1029-1038.
Garcia-Echeverria, C., and Sellers, W. R. (2008). Oncogene 27, 5511-5526.
Guertin, D. A., et al. (2006). Developmental cell 11, 859-871.
Hibi, M., et al. (1993). Genes & development 7, 2135-2148.
Inoki, K., et al. (2002). Nature cell biology 4, 648-657.
Kharas, M. G., et al. (2010a). Nature medicine 16, 903-908.
Kharas, M. G., et al (2010b). Blood 115, 1406-1415.
Kim, A. H., et al. (2001). Molecular and cellular biology 21, 893-901.
Kops, G. J., and Burgering, B. M. (2000). Journal of anatomy 197 Pt 4, 571-574.
Kops, G. J., et al. (1999). Nature 398, 630-634.
Krivtsov, A. V., et al. (2006). Nature 442, 818-822.
Lee, J. Y., et al. (2010). Cell stem cell 7, 593-605.
Liu, P., et al. (2009). Nat Rev Drug Discov 8, 627-644.
Martelli, A. M., et al. (2009). Expert opinion on investigational drugs 18, 1333-1349.
Miyamoto, K., et al. (2007). Cell stem cell 1, 101-112.
Naka, K., et al. (2010). Nature 463, 676-680.
Nicholson, K. M., and Anderson, N. G. (2002). Cellular signalling 14, 381-395.
Paik, J. H., et al. (2007). Cell 128, 309-323.
Park, S., (2010). Haematologica, 95, 819-828.
Sancak, Y., et al. (2007). Leukemia research 33, 1706-1709.
Sarbassov, D. D., et al. (2005). Science 307, 1098-1101.
Stephens, L., et al. (1998). Science 279, 710-714.
Tamburini, J., et al. (2007). Blood 110, 1025-1028.
Tee, A. R., et al. (2003). Curr Biol 13, 1259-1268.
Tothova, Z., et al. (2007). Cell 128, 325-339.
Wang, L., et al. (2007). J. Biol. Chem. 282, 20036-20044.
Wang, M. C., et al. (2005). Cell 121, 115-125.
Wang, Z., et al. (2008). Nature 455, 1205-1209.
Yalcin, S., et al. (2008). J. Biol. Chem. 283, 25692-25705.
Yilmaz, O. H., et al. (2006). Nature 441, 475-482.

What is claimed:

1.-23. (canceled)

24. A method of treating leukemia in a mammal in need thereof, the method comprising administering an effective amount of an agent that inhibits the activity or expression of a FOXO transcription factor in the mammal, whereby leukemia is treated in said mammal.

25. A method of inhibiting leukemia initiating cells (LICs) in a mammal in need thereof, the method comprising administering an effective amount of an agent that inhibits the activity or expression of a FOXO transcription factor in the mammal, wherein said mammal has been diagnosed with leukemia and whereby the LICs are inhibited in said mammal.

26. The method of claim 24, wherein the FOXO transcription factor is FOXO 1, FOXO 3, FOXO 4 or FOXO 6.

27. The method of claim 24, wherein the agent is selected from the group consisting of a small molecule, an oligonucleotide or an RNAi agent.

28. (canceled)

29. (canceled)

30. (canceled)

31. The method of claim 24, wherein the agent is an RNAi agent that inhibits the expression of the FOXO transcription factor in the mammal.

32. The method of claim 31, wherein the RNAi agent comprises a double-stranded ribonucleic acid (dsRNA) wherein the dsRNA comprises a sense strand and an antisense strand, the antisense strand comprising a region of complementarity to and having at least 15 contiguous nucleotides differing by no more than 3 nucleotides from a molecule selected from the group consisting of SEQ ID NOs:11-22.

33. The method of claim 32, wherein the RNAi agent comprises the nucleotide sequence of one of SEQ ID NOs: 11-14.

34. The method of claim 24, wherein the agent is a vector comprising a nucleic acid sequence that is at least 90% identical to one of SEQ. ID. Nos. 11-22.

35. (canceled)

36. The method of claim 24, wherein the mammal is a human.

37. The method of claim 24, further comprising the steps, before said administering step, of measuring the expression or activity of a FOXO transcription factor, and comparing the measured expression or activity of said FOXO transcription factor to a standard, wherein if the level of FOXO expression or activity is above the level of said standard, one proceeds to said administering step.

38. The method of claim 24, further comprising administering an effective amount of an agent that increases the expression or activity of the AKT protein kinase in the mammal.

39. The method of claim 38, wherein the agent that increases the expression or activity of the AKT protein kinase is selected from the group consisting of a small molecule, an aptamer, a peptide, a protein, and an oligonucleotide.

40. The method of claim 38, wherein the agent that increases the expression or activity of the AKT protein kinase is a vector comprising a sequence encoding an AKT protein kinase.

41. The method of claim 40, wherein the vector is an expression vector comprising a nucleic acid sequence that is at least 95% identical to SEQ. ID. No. 23.

42. (canceled)

43. The method of claim 24 further comprising administering an effective amount of an agent that inhibits the expression or activity of a JNK protein kinase in the mammal.

44. (canceled)

45. The method of claim 24, further comprising administering an effective amount of rapamycin.

46. The method of claim 24, wherein the leukemia is acute myelogenous leukemia.

47. A method of treating leukemia in a mammal in need thereof, the method comprising measuring the expression or activity of a FOXO transcription factor, and comparing the measured expression or activity of said FOXO transcription factor to a standard, wherein if the level of FOXO expression or activity is below the level of said standard, one proceeds to administer an effective amount of an agent that inhibits the expression or activity of a JNK protein kinase in a mammal, or rapamycin or a combination thereof, whereby leukemia is treated in said mammal.

48. The method of claim 47, further comprising measuring the expression or activity of AKT protein kinase, and comparing the measured expression or activity of said AKT protein kinase to a standard, wherein if the level of AKT protein kinase expression or activity is above the level of said standard, one proceeds to administer an effective amount of an agent that inhibits the expression or activity of the JNK protein kinase in a mammal and an effective amount of rapamycin.

49. A method of inhibiting leukemia initiating cells (LICs) in a mammal in need thereof, the method comprising measuring the expression or activity of a FOXO transcription factor, and comparing the measured expression or activity of said FOXO transcription factor to a standard, wherein if the level of FOXO expression or activity is below the level of said standard, one proceeds to administer an effective amount of an agent that inhibits the expression or activity of a JNK protein kinase in a mammal, or rapamycin or a combination thereof, wherein said mammal has been diagnosed with leukemia and whereby the LICs are inhibited in said mammal.

50. The method of claim 49, further comprising measuring the expression or activity of AKT protein kinase, and comparing the measured expression or activity of said AKT protein kinase to a standard, wherein if the level of AKT protein kinase expression or activity is above the level of said standard, one proceeds to administer an effective amount of an agent that inhibits the expression or activity of the JNK protein kinase in a mammal and an effective amount of rapamycin.

51. (canceled)

52. The method of claim 48, wherein the agent that increases the expression or activity of the AKT protein kinase is selected from the group consisting of a small molecule, an aptamer, a peptide, a protein, and an oligonucleotide.

53. The method of claim 52, wherein the agent that increases the expression or activity of the AKT protein kinase is a vector comprising a sequence encoding an AKT protein kinase.

54. The method of claim 53, wherein the vector is an expression vector comprising a nucleic acid sequence that is at least 95% identical to SEQ. ID. No. 23.

55. (canceled)

56. The method of claim 47, wherein the leukemia is acute myelogenous leukemia.

57. The method of claim 47, wherein the FOXO transcription factor is FOXO 1, FOXO 3, FOXO 4 or FOXO 6.


SEQUENCE LISTING (26 sequences)

SEQ ID NO: 1 - DNA, 5738 bp, Homo sapiens
gcagccgccacattcaacaggcagcagcgcagcgggcgcgccgctggggagagcaagcggcccgcggcgtccgtccgtccttccgtccgcggccctgtcagctggagcgcggcgcaggctctgccccggcccggcggctctggccggccgtccagtccgtgcggcggaccccgaggagcctcgatgtggatggccccgcgaagttaagttctgggctcgcgcttccactccgccgcgccttcctcccagtttccgtccgctcgccgcaccggcttcgttcccccaaatctcggaccgtcccttcgcgccccctccccgtccgcccccagtgctgcgttctccccctcttggctctcctgcggctgggggaggggcgggggtcaccatggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcatctgcttctactgaactatgcgtactgcatagcatcaagtcttctctagagacctctgtagtcctgggaggcctcataatgtttgtagatcagaaaagggagatctgcatctaaagcaatggtcctttgtcaaacgagggattttgatccacttcaccattttgagttgagctttagcaaaagtttcccctcataattctttgctcttgtttcagtccaggtggaggttggttttgtagttctgccttgaggaattatgtcaacactcatacttcatctcattctcccttctgccctgcagattagattacttagcacactgtggaagtttaagtggaaggagggaatttaaaaatgggacttgagtggtttgtagaatttgtgttcataagttcagatgggtagcaaatggaatagaacttacttaaaaattggggagatttatttgaaaaccagctgtaagttgtgcattgagattatgttaaaagccttggcttaagaatttgaaaatttctttagcctgtagcaacctaaactgtaattcctatcattatgttttattactttccaattacctgtaactgacagaccaaattaattggctttgtgtcctatttagtccatcagtattttcaagtcatgtggaaagcccaaagtcatcacaatgaagagaacaggtgcacagcactgttcctcttgtgttcttgagaaggatctaatttttctgtatatagcccacatcacacttgctttgtcttgtatgttaattgcatcttcattggcttggtatttcctaaatgtttaacaagaacacaagtgttcctgataagatttcctacagtaagccagctctattgtaagcttcccactgtgatgatcatttttttgaagattcattgaacagccaccactctatcatcctcattttggggcagtccaagacatagctggttttagaaacccaagttcctctaagcacagcctcccgggtatgtaactgaacttggtgccaaagtacttgtgtactaatttctattactacgtactgtcactttcctcccgtgccattactgcatcataatacaaggaacctcagagcccccatttgttcattaaagaggcaactacagccaaaatcactgttaaaatcttactacttcatggagtagctcttaggaaaatatatcttcctcctgagtctgggtaattatacctctcccaagcccccattgtgtgttgaaatcctgtcatgaatccttggtagctctctgagaacagtgaagtccagggaaaggcatctggtctgtctggaaagcaaacattatgtggcctctggtagtttttttcctgtaagaatactgactttctggagtaatgagtatatatcagttattgtacatgattgctttgtgaaatgtgcaaatgatatcacctatgcagccttgtttgatttattttctctggtttgtactgttattaaaagcatattgtattatagagctattcagatattttaaatataaagatgtattgtttccgtaatatagacgtatggaatatatttaggtaatagatgtattacttggaaagttctgctttgacaaactgacaaagtctaaatgagcacatgtatcccagtgagcagtaaatcaatggaacatcccaagaagaggataaggatgcttaaaatggaaatcattctccaacgatatacaaattggacttgttcaactgctggatatatgctaccaataaccccagccccaacttaaaattcttacattcaagctcctaagagttcttaatttataactaattttaaaagagaagtttcttttctggttttagtttgggaataatcattcattaaaaaaaatgtattgtggtttatgcgaacagaccaacctggcattacagttggcctctccttgaggtgggcacagcctggcagtgtggccaggggtggccatgtaagtcccatcaggacgtagtcatgcctcctgcatttcgctacccgagtttagtaacagtgcagattccacgttcttgttccgatactctgagaagtgcctgatgttgatgtacttacagacacaagaacaatctttgctataattgtataaagccataaatgtacataaattatgtttaaatggcttggtgtctttcttttctaattatgcagaataagctctttattaggaattttttgtgaagctattaaatacttgagttaagtcttgtcagccacaa

SEQ ID NO: 2 - DNA, 7341 bp, Homo sapiens
gcgcgaggccgtcgattcgctcgcggctccatcgcggcctggccggggggcggtgtctgctgcgccaggttcgctggccgcacgtcttcaggtcctcctgttcctgggaggcgggcgcggcaggactgggaggtggcggcagcgggcgaggactcgccgaggacggggctccggcccgggataaccaactctccttctctcttctttggtgcttccccaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttccttgtacagggtggtgagccgcctgccagtggaggacagcacccctcagcaccacccaccctcattcagagcacaccgtgagcccccgtcggccattctgtggtgttttaatattgcgatggtttatgggacgttttaagtgttgttcttgtgtttgttttcctttgactttctgagtttttcacatgcattaacttgcggtatttttctgttaaaatgttaaccgtccttcccctagcaaatttaaaaacagaaagaaaatgttgtaccagttaccattccgggttcgagcatcacaagcttttgagcgcatggaactccataaactaacaaattacataaactaaagggggattttctttcttcttttgtttggtagaaaattatccttttctaaaaactgaacaatggcacaattgtttgctatgtgcacccgtccaggacagaaccgtgcataggcaaaaggagtggagcacagcgtccggcccagtgtgtttccggttctgagtcagggtgatctgtggacgggaccccagcaccaagtctacgggtgccagatcagtagggcctgtgatttcctgtcagtgtcctcagctaatgtgaacagtgttggtctgctggttagaaactagaatattgatattttcaggaaagaaatcagctcagctctccactcattgccaaatgtcactaaagggtttagttttaaggagaaagaaaaggaaaaaaaaaaaaaacaaaaaagtcctgttttgctttgcagaacaaatgaacttacaggtgagcattaagcttgcagtgagaaatgtgcgaagagtaaaaacccaagtcaatgctgaggcagttctaacttcactgttttcctaaatacacatccttgattattttcagccttgctatataatctgatctgctagaagtgtatgagtgagaggcaatagcatacaaactgattttttaaatataagcttaggttgtaattgtacaagtgactcaatggaagtacaaaatagggcagttttaacttttttttctgcttctatggatttcattttgttgtgttttcaaaaagttatggtgctgtataggtgctttctgtttaacctggaaagtgtgattatattcgttaccttctttggtagacggaatagttgggaccacctttggtacataagaaattggtataacgatgctctgattagcacagtatatgcatacttctccaaagtgatatatgaagactcttttctttgcataaaaagcattaggcatataaatgtataaatatattttatcatgtacagtacaaaaatggaaccttatgcatgggccttaggaatacaggctagtatttcagcacagacttccctgcttgagttcttgctgatgcttgcaccgtgacagtgggcaccaacacagacgtgccacccaaccccctgcacacaccaccggccaccaggggcccccttgtgcgccttggctttataactcctctgggggtgatattggtggtgatcacagctcctagcataatgagagttccatttggtattgtcacacgtctcctgcctcgcttgggttgccatgtttgagcgatggccctgttgatttcaccctgccttttactgaatctgtaaattgttgtgcaattgtggttatagtagactgtagcacattgccttttctaaactgctacatgtttataatcttcatttttaaagtatgtgtaatttttttaagtatgtattctattcatatggtctgcttgtcagtgagccagacttgcttactatattcctttataataatgctagccacttcctggattctttagtaatgtgctgtatgcaagaactttccagtagcagtgaaggagggttgcctctccaagcttcctaagggatgctgccctgtgtggggatgcattgcagaggcactagtagcatgggggctagagtggggagcgagatgtaaaagggtggggggataggagaattccagagtgcttccagcattagggtcctgagaacttctgagttcagagaaacatgcaaagtgactaacaaaatagctacttacctttgcagttttacagaccctgggagctgctttgggagtgagaaaggcaaccctccaatgtgtttcaactttaaaatgttgaattcttttcagacatggtatctcatttattctccttttctagcgtttgttgaatttcaggcagaatgtcttacagaatgtcctagaaccagattatcatttaatctgaaacagctgaggaagggacagagaaggtacaagggcaaggcagcacaaaacagatcaggagaatgaagagggaatgctttggttttttgttttgttttgttttttctttttcaagtaactaaaacagcatctacatgtagagtgttgtggagagctgagaccagggtaaagtcaagtgcagcatcagtactgcgagacccaccagcccctggagagggtcagccgagaatctggtagtgaagcctgtctagggtcccggcaccctcaccctcagccacctgcagagaggccagggccccagagactagcctggttctgaagtgggcaggggtgctgccagagccctctgccccttatgttgagaccctgctttcaggacaggccagccgttggccaccatgtcacattctgagtgagtgtcacaggtccctaacaataattttctgatctggagcatatcagcagaatgcttagcctcaaggggcctggcagctgtaatgtttgatttatgatgagaactatccgaggccacccttggcctctaaataagctgctctagggagccgcctactttttgatgagaaattagaagagtacctaatgttgaaaacatgacatgcgctcttgggatctgctgttctctccagggctccagaacctgatacctgttaccaaagctaggaaagagctttatcacaagccttcactgtcctggcatgagaactggctgccaggctcagtgtaccccattaactgtgaatgaatctgagcttggtttcctttattgcttcctctgcaatatgattgctgaaacacattttaaaaattcagaagcttgtcactcctgttaatgggaggatcagtcacacatgtgtagtacaaggcggactttgtgtttgtttttggtgttaatttttagcattgtgtgtgttgcttccccaccctgaggagaggacaccatggcttactactcaggacaagtatgccccgctcagggtgtgatttcaggtggcttccaaacttgtacgcagtttaaagatggtggggacagactttgcctctacctagtgaaccccacttaaagaataaggagcatttgaatctcttggaaaaggccatgaagaataaagcagtcaaaaagaagtcctccatgttggtgccaaggacttgcgaggggaaataaaaatgttatccagcctgaccaacatggagaaaccccgtctccattaaaaatacaaaattagcctggcatggtggcgcatgcctgtaatcccagctactctggaggctgaggcaggagaatcgcttgaacccaggaggcggaggtcgcagtgagccgagatcatgccagtgcactccagcctgggtaacaagagtgaaactccgtgtcaaaaaaaaaaaaaaaatgttactcatcctctctgaaagcaaaaaggaaaccctaacagctctgaactctggttttatttttcttgctgtatttgggtgaacattgtatgattaggcataatgttaaaaaaaaaaatttttttttggtagaaatgcaatcaccagtaaagaggtacgaaaaagctagcctctctcagagaccggggaggcagagtactactagaggaagtgaagttctgatggaatcatgcctgtcaaatgaggtcttgaagcggatgcccaaataaaagagtatattttatctaaatcttaagtgggtaacattttatgcagtttaaatgaatggaatattttcctcttgtttagttgtatctgtttgtatttttctttgatgaatgattggtcatgaggcctcttgccacactccagaaatacgtgtgcggctgcttttaagaactatgtgtctggtcacttatttctctaaaattatctcattgcctggcaatcagtcttctcttgtatacttgtcctagcacattatgtacatgggaaatgtaaacaaatgtgaaggaggaccagaaaaattagttaatatttaaaaaaatgtattgtgcattttggcttcacatgtttaactttttttaagaaaaaagttgcatgaatggaaaaaaaaatctgtatacagtatctgtaaaaactatcttatctgtttcaattccttgctcatatcccatataatctagaactaaatatggtgtgtggccatatttaaacacctgagagtcaagcagttgagactttgatttgaagcacctcatccttctttcaatgcgaacactatcatatggcattcttactgaggattttgtctaaccatatgttgccatgaattaactctgccgcctttcttaaggatcaaaaccagtttgatttgggaatcttcccctttccaaatgaaatagagatgcagtacttaactttccttggtgtttgtagatattgccttgtgtattccacttaaaaccgtaatctagtttgtaaaagagatggtgacgcatgtaaataaagcatcagtgacactct

SEQ ID NO: 3 - DNA, 7314 bp, Homo sapiens
ctgctcgtggaagggaggaggaggaatgtggaaggtggcggcgcgcgagctgacaggcggttcctcgggcgggctgcggcgacggcccgagcgcttctccttcgccgaggttgcgcgccccctcctcgccgtgccccgggcccgcgcgggatcgtgcgtcgtcccgccgcgtccctgaagggaaggaagcgaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttccttgtacagggtggtgagccgcctgccagtggaggacagcacccctcagcaccacccaccctcattcagagcacaccgtgagcccccgtcggccattctgtggtgttttaatattgcgatggtttatgggacgttttaagtgttgttcttgtgtttgttttcctttgactttctgagtttttcacatgcattaacttgcggtatttttctgttaaaatgttaaccgtccttcccctagcaaatttaaaaacagaaagaaaatgttgtaccagttaccattccgggttcgagcatcacaagcttttgagcgcatggaactccataaactaacaaattacataaactaaagggggattttctttcttcttttgtttggtagaaaattatccttttctaaaaactgaacaatggcacaattgtttgctatgtgcacccgtccaggacagaaccgtgcataggcaaaaggagtggagcacagcgtccggcccagtgtgtttccggttctgagtcagggtgatctgtggacgggaccccagcaccaagtctacgggtgccagatcagtagggcctgtgatttcctgtcagtgtcctcagctaatgtgaacagtgttggtctgctggttagaaactagaatattgatattttcaggaaagaaatcagctcagctctccactcattgccaaatgtcactaaagggtttagttttaaggagaaagaaaaggaaaaaaaaaaaaaacaaaaaagtcctgttttgctttgcagaacaaatgaacttacaggtgagcattaagcttgcagtgagaaatgtgcgaagagtaaaaacccaagtcaatgctgaggcagttctaacttcactgttttcctaaatacacatccttgattattttcagccttgctatataatctgatctgctagaagtgtatgagtgagaggcaatagcatacaaactgattttttaaatataagcttaggttgtaattgtacaagtgactcaatggaagtacaaaatagggcagttttaacttttttttctgcttctatggatttcattttgttgtgttttcaaaaagttatggtgctgtataggtgctttctgtttaacctggaaagtgtgattatattcgttaccttctttggtagacggaatagttgggaccacctttggtacataagaaattggtataacgatgctctgattagcacagtatatgcatacttctccaaagtgatatatgaagactcttttctttgcataaaaagcattaggcatataaatgtataaatatattttatcatgtacagtacaaaaatggaaccttatgcatgggccttaggaatacaggctagtatttcagcacagacttccctgcttgagttcttgctgatgcttgcaccgtgacagtgggcaccaacacagacgtgccacccaaccccctgcacacaccaccggccaccaggggcccccttgtgcgccttggctttataactcctctgggggtgatattggtggtgatcacagctcctagcataatgagagttccatttggtattgtcacacgtctcctgcctcgcttgggttgccatgtttgagcgatggccctgttgatttcaccctgccttttactgaatctgtaaattgttgtgcaattgtggttatagtagactgtagcacattgccttttctaaactgctacatgtttataatcttcatttttaaagtatgtgtaatttttttaagtatgtattctattcatatggtctgcttgtcagtgagccagacttgcttactatattcctttataataatgctagccacttcctggattctttagtaatgtgctgtatgcaagaactttccagtagcagtgaaggagggttgcctctccaagcttcctaagggatgctgccctgtgtggggatgcattgcagaggcactagtagcatgggggctagagtggggagcgagatgtaaaagggtggggggataggagaattccagagtgcttccagcattagggtcctgagaacttctgagttcagagaaacatgcaaagtgactaacaaaatagctacttacctttgcagttttacagaccctgggagctgctttgggagtgagaaaggcaaccctccaatgtgtttcaactttaaaatgttgaattcttttcagacatggtatctcatttattctccttttctagcgtttgttgaatttcaggcagaatgtcttacagaatgtcctagaaccagattatcatttaatctgaaacagctgaggaagggacagagaaggtacaagggcaaggcagcacaaaacagatcaggagaatgaagagggaatgctttggttttttgttttgttttgttttttctttttcaagtaactaaaacagcatctacatgtagagtgttgtggagagctgagaccagggtaaagtcaagtgcagcatcagtactgcgagacccaccagcccctggagagggtcagccgagaatctggtagtgaagcctgtctagggtcccggcaccctcaccctcagccacctgcagagaggccagggccccagagactagcctggttctgaagtgggcaggggtgctgccagagccctctgccccttatgttgagaccctgctttcaggacaggccagccgttggccaccatgtcacattctgagtgagtgtcacaggtccctaacaataattttctgatctggagcatatcagcagaatgcttagcctcaaggggcctggcagctgtaatgtttgatttatgatgagaactatccgaggccacccttggcctctaaataagctgctctagggagccgcctactttttgatgagaaattagaagagtacctaatgttgaaaacatgacatgcgctcttgggatctgctgttctctccagggctccagaacctgatacctgttaccaaagctaggaaagagctttatcacaagccttcactgtcctggcatgagaactggctgccaggctcagtgtaccccattaactgtgaatgaatctgagcttggtttcctttattgcttcctctgcaatatgattgctgaaacacattttaaaaattcagaagcttgtcactcctgttaatgggaggatcagtcacacatgtgtagtacaaggcggactttgtgtttgtttttggtgttaatttttagcattgtgtgtgttgcttccccaccctgaggagaggacaccatggcttactactcaggacaagtatgccccgctcagggtgtgatttcaggtggcttccaaacttgtacgcagtttaaagatggtggggacagactttgcctctacctagtgaaccccacttaaagaataaggagcatttgaatctcttggaaaaggccatgaagaataaagcagtcaaaaagaagtcctccatgttggtgccaaggacttgcgaggggaaataaaaatgttatccagcctgaccaacatggagaaaccccgtctccattaaaaatacaaaattagcctggcatggtggcgcatgcctgtaatcccagctactctggaggctgaggcaggagaatcgcttgaacccaggaggcggaggtcgcagtgagccgagatcatgccagtgcactccagcctgggtaacaagagtgaaactccgtgtcaaaaaaaaaaaaaaaatgttactcatcctctctgaaagcaaaaaggaaaccctaacagctctgaactctggttttatttttcttgctgtatttgggtgaacattgtatgattaggcataatgttaaaaaaaaaaatttttttttggtagaaatgcaatcaccagtaaagaggtacgaaaaagctagcctctctcagagaccggggaggcagagtactactagaggaagtgaagttctgatggaatcatgcctgtcaaatgaggtcttgaagcggatgcccaaataaaagagtatattttatctaaatcttaagtgggtaacattttatgcagtttaaatgaatggaatattttcctcttgtttagttgtatctgtttgtatttttctttgatgaatgattggtcatgaggcctcttgccacactccagaaatacgtgtgcggctgcttttaagaactatgtgtctggtcacttatttctctaaaattatctcattgcctggcaatcagtcttctcttgtatacttgtcctagcacattatgtacatgggaaatgtaaacaaatgtgaaggaggaccagaaaaattagttaatatttaaaaaaatgtattgtgcattttggcttcacatgtttaactttttttaagaaaaaagttgcatgaatggaaaaaaaaatctgtatacagtatctgtaaaaactatcttatctgtttcaattccttgctcatatcccatataatctagaactaaatatggtgtgtggccatatttaaacacctgagagtcaagcagttgagactttgatttgaagcacctcatccttctttcaatgcgaacactatcatatggcattcttactgaggattttgtctaaccatatgttgccatgaattaactctgccgcctttcttaaggatcaaaaccagtttgatttgggaatcttcccctttccaaatgaaatagagatgcagtacttaactttccttggtgtttgtagatattgccttgtgtattccacttaaaaccgtaatctagtttgtaaaagagatggtgacgcatgtaaataaagcatcagtgacactct

SEQ ID NO: 4 - DNA, 3200 bp, Homo sapiens
aaaagggggagggaactgcggctaaggagacgttcggtgatgggagcgcaatatatgaggggatacagtgcctcaggtttaaaagagcaggaagctgagtgagaggttgcagaaaaagtgtcttcgctcggcagaggttacaggtggcatctcagaaagagctttgaggctacaggctgtagtcgggaaggggatcggagaactgtgtgaagggacagcttagggactagcgtcctgggactagggggaagttcgcgactttctgaagactggcaggaatgtgcctcctggccctcgatgcttcccccctgaggggaggcatcgtgagggactgtggcaggcttcactgaacgctgagccggggaggtccaactccacgtatggatccggggaatgagaattcagccacagaggctgccgcgatcatagacctagatcccgacttcgaaccccagagccgtccccgctcctgcacctggccccttccccgaccagagatcgctaaccagccgtccgagccgcccgaggtggagccagatctgggggaaaaggccattgaaagcgccccggagaagcgactgacacttgcccagatctacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctccaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctgagtcatgcctggaagctttgtcccctgcttcagatgtggagccaggcgtgttcatatctactctttacccttgagccctccccaggaatttgggaccctgctttagagctagggtggggtctggtcacacacaggtgttgaagaaattataaagataaagctgccccatctggggacgatatggggagggagatgggaggggaaaggggagagggtttttctcactgtgccaattagggggtaaggccccctctcaggagccatcatcggctttccccattcctacccacttaggctttgtagcaagatgagcaatgctgttggaaatgtgaagtcaccagtggccttacccctgcctttgggagcaggatttttttgtagagagtcttatctgagctgagccaggctagctggagcctgggatttctatgcagtggccccttaggccagtgatgtgcggtgggtgggctgtttaggggatctggaagggccaaggtctgagcactggagtggctcgccaggccaaatcacccttagaaggctgcagataacagaaaggctttttataaacttttaaagaaatataaacacaaatatagagattttttaaccatggcagggtgctagtggtgggcagaatgcttttttttctttctgaaggctttgtgatagtgacatgatacaaacactacagacaataaatattaggagacacagggaagtggggagaggtggggagtaatagtaaacacagggaagagctcccctacggaccaggtatagagaaaggtctatgcagaaataggttagagtttccctaacaaaaaagctaacccaggtcccctcattccttcaacttgtgcctgggagtgtgtggtgttagggtgcagccacactcttctatgacccagcatgggttagtgctatggtgggagagtacattgaaggcctggaattagcttggggccagggaagggactgggaggggagagaagagaaggagggaaggatttaggatggtaaagttaggtacagagacctccctgttcaaggcccctgacagctgtccctgcccttcttccccttccctgactgcaggggttatgtggaagtgtgtgtggcagcaggcagcggggaggggaggaacagggaagggggagctggggagcttggctgagggtctgggaaatgagcagggatggggggggatgtggatcaggtttactagcacctgccagggaggccatctggggctccttctccaccccagcccccaaagcagcccttcccccagtgccctttgcatcgtcccctcccccacccctgctgtgggttcccatcatttcctgtgtcagcgcctggcctacccagattgtatcatgtgctagattggagtggggaagtgtgtcaaatcaataaatgaataaattcaataaatgcctataaccagcaaaaaaaaaaaaaaaaa

SEQ ID NO: 5 - DNA, 3365 bp, Homo sapiens
aaaagggggagggaactgcggctaaggagacgttcggtgatgggagcgcaatatatgaggggatacagtgcctcaggtttaaaagagcaggaagctgagtgagaggttgcagaaaaagtgtcttcgctcggcagaggttacaggtggcatctcagaaagagctttgaggctacaggctgtagtcgggaaggggatcggagaactgtgtgaagggacagcttagggactagcgtcctgggactagggggaagttcgcgactttctgaagactggcaggaatgtgcctcctggccctcgatgcttcccccctgaggggaggcatcgtgagggactgtggcaggcttcactgaacgctgagccggggaggtccaactccacgtatggatccggggaatgagaattcagccacagaggctgccgcgatcatagacctagatcccgacttcgaaccccagagccgtccccgctcctgcacctggccccttccccgaccagagatcgctaaccagccgtccgagccgcccgaggtggagccagatctgggggaaaaggtacacacggaggggcgctcagagccgatcctgttgccctctcggctcccagagccggccgggggcccccagcccggaatcctgggggctgtaacaggtcctcggaagggaggctcccgccggaatgcctggggaaatcagtcatatgcagaactcatcagccaggccattgaaagcgccccggagaagcgactgacacttgcccagatctacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctccaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctgagtcatgcctggaagctttgtcccctgcttcagatgtggagccaggcgtgttcatatctactctttacccttgagccctccccaggaatttgggaccctgctttagagctagggtggggtctggtcacacacaggtgttgaagaaattataaagataaagctgccccatctggggacgatatggggagggagatgggaggggaaaggggagagggtttttctcactgtgccaattagggggtaaggccccctctcaggagccatcatcggctttccccattcctacccacttaggctttgtagcaagatgagcaatgctgttggaaatgtgaagtcaccagtggccttacccctgcctttgggagcaggatttttttgtagagagtcttatctgagctgagccaggctagctggagcctgggatttctatgcagtggccccttaggccagtgatgtgcggtgggtgggctgtttaggggatctggaagggccaaggtctgagcactggagtggctcgccaggccaaatcacccttagaaggctgcagataacagaaaggctttttataaacttttaaagaaatataaacacaaatatagagattttttaaccatggcagggtgctagtggtgggcagaatgcttttttttctttctgaaggctttgtgatagtgacatgatacaaacactacagacaataaatattaggagacacagggaagtggggagaggtggggagtaatagtaaacacagggaagagctcccctacggaccaggtatagagaaaggtctatgcagaaataggttagagtttccctaacaaaaaagctaacccaggtcccctcattccttcaacttgtgcctgggagtgtgtggtgttagggtgcagccacactcttctatgacccagcatgggttagtgctatggtgggagagtacattgaaggcctggaattagcttggggccagggaagggactgggaggggagagaagagaaggagggaaggatttaggatggtaaagttaggtacagagacctccctgttcaaggcccctgacagctgtccctgcccttcttccccttccctgactgcaggggttatgtggaagtgtgtgtggcagcaggcagcggggaggggaggaacagggaagggggagctggggagcttggctgagggtctgggaaatgagcagggatggggggggatgtggatcaggtttactagcacctgccagggaggccatctggggctccttctccaccccagcccccaaagcagcccttcccccagtgccctttgcatcgtcccctcccccacccctgctgtgggttcccatcatttcctgtgtcagcgcctggcctacccagattgtatcatgtgctagattggagtggggaagtgtgtcaaatcaataaatgaataaattcaataaatgcctataaccagcaaaaaaaaaaaaaaaaa

SEQ ID NO: 6 - DNA, 1770 bp, Homo sapiens
atggctgcgaagctgcgagcgcatcaggtggacgtggacccggacttcgcgccgcagagccggccgcgctcgtgtacctggcccctgccgcagcctgacttggccggcgacgaggacggagcgctgggcgcaggggtggccgagggcgccgaggactgcgggccggagcgccgggctacggccccggcgatggccccagcgccgcccctgggcgcggaggtcggaccgctgcggaaagcgaagagctctcggcggaacgcgtgggggaacctgtcctacgccgacctcatcaccaaagccatcgagagcgccccggacaagcggctcacgctctcgcagatctacgactggatggtccgttacgtgccctacttcaaggataaaggcgacagcaacagctcggccggctggaaggtgggggcgtccggctggggagggggctggggacgccgactggggcttccaggggctgctaggtgtgttgggctccaggagggccgtcagaaggagggctttttggcaccccccggggtcacagagtcctgctccagcagggctccctcctcctcctttgtcctccggggatccccagccccaagggctgatgggggtggggaagtgactttccctgaggtcgctacctgtgaagtagtggacctgggaattggaccccaagctcgagctttttctgttccattgggagccagcctgcagggctggggcacagctgctgggccctccccagagctgggctcagggcagggctggctgcccttctatcccttctccactgcagaccctagtgccgaggatttgcacatggcctcgggtccctggcatcatccaggtcacatcttttgccaggaagaggcctggactgagaacctgaacttcaccccagatggcagggcaaatggtctccctcgggccctgaatcagggtctccagagaaccaggcagggcaaggctggcaagtcccagctgttgccttcaaaggccagaaagtgccagagacagggagcctggggccctgaggagtcagatcagtgcctggagctggggcagtacctggaccaggaagttgaggccaccagaacaggcctggcaaaagcaggcagtgacatggggagcactggagactggcacagcagaattaaaaagaggatgtatggagagatggcagaggtcttctggcgtgtgaatggggactttgccacacactgtggggattttgagaaggctccaacagcaaggggcttggagggcaggcaggtggtaccgccgccgccgcccgcgcccaggcccggcccggtgctgggtgcgccgggggagctggcgctggcgggcgcagccgccgcctaccccggcaaaggggcggccccgtacgcgccgcccgcgccctcgcgcagtgccttagcccaccccatcagccttatgacgctgcccggcgaggcgggcgccgcgggcctggcaccgccgggccacgccgccgccttcgggggcccgcccggcggcctcctgctggacgctctgccggggccctacgctgccgccgccgccgggccgctgggcgccgcgcccgaccgcttcccggccgacctggacctcgacatgttcagcgggagcctcgagtgcgacgtggagtccatcatcctcaacgacttcatggacagcgacgaaatggacttcaacttcgattcggccctgcctccgccgccgccgggcctggccggggccccgccccccaaccagagctgggtgccgggctga

SEQ ID NO: 7 - DNA, 1479 bp, Homo sapiens
atggctgcgaagctgcgagcgcatcaggtggacgtggacccggacttcgcgccgcagagccggccgcgctcgtgtacctggcccctgccgcagcctgacttggccggcgacgaggacggagcgctgggcgcaggggtggccgagggcgccgaggactgcgggccggagcgccgggctacggccccggcgatggccccagcgccgcccctgggcgcggaggtcggaccgctgcggaaagcgaagagctctcggcggaacgcgtgggggaacctgtcctacgccgacctcatcaccaaagccatcgagagcgccccggacaagcggctcacgctctcgcagatctacgactggatggtccgttacgtgccctacttcaaggataaaggcgacagcaacagctcggccggctggaagaactccatccggcacaacctgtcgctgcacacccgtttcatccgcgtgcagaacgagggcaccggcaagagttcgtggtggatgctgaaccccgagggcggaaagacagggaagaccccgcggcgcagggccgtgtccatggacaacggggccaagttcctgcgcatcaagggcaaggcgagcaagaagaagcagctgcaggcgcccgagcgaagcccggacgacagctccccgagtgcgcccgccccggggccggtgcctgccgcagccaagtgggccgccagccccgcctcgcacgccagcgacgactacgaggcttgggccgacttccgcggcggcgggagacccctgctcggggaggcggccgagctggaggacgacgaggccctggaggccctggcgccatcatcgccgctcatgtacccaagccccgccagcgcgctgtcgccggcgctgggctcgcgctgtccgggtgagctgccccgcctggccgagctgggaggcccgctgggcctgcacggcggcggcggcgcggggctgcccgagggcctgctggacggcgcgcaggacgcgtacgggccgcggcccgcgcccaggcccggcccggtgctgggtgcgccgggggagctggcgctggcgggcgcagccgccgcctaccccggcaaaggggcggccccgtacgcgccgcccgcgccctcgcgcagtgccttagcccaccccatcagccttatgacgctgcccggcgaggcgggcgccgcgggcctggcaccgccgggccacgccgccgccttcgggggcccgcccggcggcctcctgctggacgctctgccggggccctacgctgccgccgccgccgggccgctgggcgccgcgcccgaccgcttcccggccgacctggacctcgacatgttcagcgggagcctcgagtgcgacgtggagtccatcatcctcaacgacttcatggacagcgacgaaatggacttcaacttcgattcggccctgcctccgccgccgccgggcctggccggggccccgccccccaaccagagctgggtgccgggctga

SEQ ID NO: 8 - DNA, 2610 bp, Homo sapiens
atcctgggacagggcacagggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctacagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattct

SEQ ID NO: 9 - DNA, 1254 bp, Homo sapiens
atgaagacggagcggccccggcccaacaccttcatcatccgctgcctgcagtggaccactgtcatcgaacgcaccttccatgtggagactcctgaggagcgggaggagtggacaaccgccatccagactgtggccgacggcctcaagaagcaggaggaggaggagatggacttccggtcgggctcacccagcgacaactcaggggccgaagagatggaggtgtccctggccaagcccaagcaccgcgtgaccatgaacgagtttgagtacctgaagctgctgggcaagggcactttcggcaaggtgatcctggtgaaggagaaggccacagcgtactacgccatgaagatcctcaagaaggaagtcatcgtggccaaggacgaggtggcccacacactcaccgagaaccgcgtccagcagaactccaggcaccccttcctcactcgcctgaagtactctttccagacccacgaccgcctctgctttgtcatggagtacgccaacgggggcgagctgttcttccacctgtcccgggagcgtgtgttcgccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttaccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcgcccagcgcgacggcctga

SEQ ID NO: 10 - DNA, 2626 bp, Mus musculus
ccgggaccagcggacggaccgagcagcgtcctgcggccggcaccgcggcggcccagatccggccagcagcgcgcgcccggacgccgctgccttcagccggccccgcccagcgcccgcccgcgggatgcggagcggcgggcgcccgaggccgcggcccggctaggcccagtcgcccgcacgcggcggcccgacgctgcggccaggccggctgggctcagcctaccgagaagagactctgatcatcatccctgggttacccctgtctctgggggccacggataccatgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggccgatggactcaagaggcaggaagaagagacgatggacttccgatcaggctcacccagtgacaactcaggggctgaagagatggaggtgtccctggccaagcccaagcaccgtgtgaccatgaacgagtttgagtacctgaaactactgggcaagggcacctttgggaaagtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgcgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggatggtgccactatgaagacattctgcggaacgccggagtacctggcccctgaggtgctggaggacaacgactacggccgtgcagtggactggtgggggctgggcgtggtcatgtatgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctctgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcctgaggcctggggcagcggctggcagctccacgctcctctgcattgccgagtccagaagccccgcatggatcatctgaacctgatgttttgtttctcggatgcgctggggaggaaccttgccagcctccaggaccaggggaggatgtttctactgtgggcagcagcctacctcccagccaggtcaggaggaaaactatcctggggtttttcttaatttatttcatccagtttgagaccacacatgtggcctcagtgcccagaacaattagattcatgtagaaaactattaaggactgacgcgaccatgtgcaatgtgggctcatgggtctgggtgggtcccgtcactgcccccattggcctgtccaccctggccgccacctgtctctagggtccagggccaaagtccagcaagaaggcaccagaagcacctccctgtggtatgctaactggccctctccctctgggcggggagaggtcacagctgcttcagccctagggctggatgggatggccagggctcaagtgaggttgacagaggaacaagaatccagtttgttgctgtgtcccatgctgttcagagacatttaggggattttaatcttggtgacaggagagcccctgccctcccgctcctgcgtggtggctcttagcgggtaccctgggagcgcctgcctcacgtgagccctctcctagcacttgtccttttagatgctttccctctcccgctgtccgtcaccctggcctgtcccctcccgccagacgctggccattgctgcaccatgtcgttttttacaacattcagcttcagcatttttactattataataagaaactgtccctccaaattcaataaaaattgcttttcaagcttgaaaaaaaaaaaaaaaaaaaaaaaaaaaaaaa

SEQ ID NO: 11 - DNA, 19 bp, Artificial Sequence
  Description of Artificial Sequence Synthetic oligonucleotide
gagcgtgccctacttcaag

SEQ ID NO: 12 - DNA, 19 bp, Artificial Sequence
  Description of Artificial Sequence Synthetic oligonucleotide
cttgaagtagggcacgctc

SEQ ID NO: 13 - DNA, 19 bp, Artificial Sequence
  Description of Artificial Sequence Synthetic oligonucleotide
tctcctaggagaagagctg

SEQ ID NO: 14 - DNA, 19 bp, Artificial Sequence
  Description of Artificial Sequence Synthetic oligonucleotide
cagctcttctcctaggaga

SEQ ID NO: 15 - DNA, 21 bp, Artificial Sequence
  Description of Artificial Sequence Synthetic oligonucleotide
cagaccctcaaactgacacaa

SEQ ID NO: 16 - DNA, 21 bp, Artificial Sequence
  Description of Artificial Sequence Synthetic oligonucleotide
ttgtgtcagtttgagggtctg

SEQ ID NO: 17 - DNA, 21 bp, Artificial Sequence
  Description of Artificial Sequence Synthetic oligonucleotide
ctcctttaacagcacggtgtt

SEQ ID NO: 18 - DNA, 21 bp, Artificial Sequence
  Description of Artificial Sequence Synthetic oligonucleotide
aacaccgtgctgttaaaggag

SEQ ID NO: 19 - DNA, 21 bp, Artificial Sequence
  Description of Artificial Sequence Synthetic oligonucleotide
tctcacctcttcccattcctt

SEQ ID NO: 20 - DNA, 21 bp, Artificial Sequence
  Description of Artificial Sequence Synthetic oligonucleotide
ggaatgggaagaggtgagatt

SEQ ID NO: 21 - DNA, 21 bp, Artificial Sequence
  Description of Artificial Sequence Synthetic oligonucleotide
agaagccgatatgtggacctt

SEQ ID NO: 22 - DNA, 21 bp, Artificial Sequence
  Description of Artificial Sequence Synthetic oligonucleotide
ggtccacatatcggcttcttt

SEQ ID NO: 23 - DNA, 2794 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 24 - DNA, 2878 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 25 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 26 - protein, 359 aa, Artificial Sequence
  Description of Artificial Sequence Synthetic polypeptide
GSSKSKPKDPSQRRMSDVGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKSETDTRYFDEEFTAQMITITPPDQDDSMECVDSERRPHFPQFSYSASSTA